(19) United States
(12) Patent Application Publication
(10) Pub. No.: US 2024/0027467 A1
(43) Pub. Date: Jan. 25, 2024

(54) Nanobody Exchange Chromatography

(71) Applicants: VIB VZW, Gent (BE); VRIJE UNIVERSITEIT BRUSSEL, Brussel (BE)

(72) Inventors: Jan Steyaert, Beersel (BE); Els Pardon, Wezemaal (BE); Alexandre Wohlkönig, Brussel (BE); Thomas Zögg, Heverlee (BE); Valentina Kalichuk, Brussel (BE); Phebe De Keyser, Kessel-Lo (BE); Baptiste Fischer, Watermaal-Bosvoorde (BE)

(21) Appl. No.: 17/786,724

(22) PCT Filed: Dec. 18, 2020
(86) PCT No.: PCT/EP2020/087291

§ 371 Date: Jun. 17, 2022

(30) Foreign Application Priority Data
Dec. 20, 2019 (EP) 19219043.7

(51) Int. Cl. G01N 33/68; G01N 33/543; G01N 1/34

(52) U.S. Cl. CPC G01N 33/6827; G01N 33/54306; G01N 1/34

(57) ABSTRACT

The present invention relates to the field of affinity purification and provides for means and methods applying protein binding agents competing for a target protein for use as capture and elution tool, wherein the elution agent comprises an immunoglobulin single variable domain (ISVD), and is capable of displacing the capturing binding agent. More specifically, the displacement efficiency of the ISVD-containing protein binding agent is driven by its dissociation kinetics, with a rate constant of dissociation (k_{off}) equal or lower as compared to the capturing agent. Furthermore, said protein binding agents are deployable in high-throughput purification from complex mixtures, or for capturing protein-complexes, thereby facilitating structural, biochemical and physicochemical analysis of said target proteins.

### CROSS-REFERENCE TO RELATED APPLICATIONS

This application is a national phase entry under 35 U.S.C. § 371 of International Patent Application PCT/EP2020/087291, filed Dec. 18, 2020, designating the United States of America and published in English as International Patent Publication WO 2021/123360 on Jun. 24, 2021, which claims the benefit under Article 8 of the Patent Cooperation Treaty to European Patent Application Serial No. 19219043.7, filed Dec. 20, 2019, the entireties of which are hereby incorporated by reference.

### FIELD OF THE INVENTION

The present invention relates to the field of affinity purification and provides for means and methods applying protein binding agents competing for a target protein for use as capture and elution tool, wherein the elution agent comprises an immunoglobulin single variable domain (ISVD), and is capable of displacing the capturing binding agent. More specifically, the displacement efficiency of the ISVD-containing protein binding agent is driven by its dissociation kinetics, with a rate constant of dissociation (k_{off}) equal or lower as compared to the capturing agent. Furthermore, said protein binding agents are deployable in high-throughput purification from complex mixtures, or for capturing protein-complexes, thereby facilitating structural, biochemical and physicochemical analysis of said target proteins.

### BACKGROUND

As an affinity-based technique, immunopurification presents some advantages over chromatographic methods based on chemical and physical properties. It can simplify the purification of proteins from complex multi-step procedures to a single step protocol, reducing costs and time. Consequently, it can improve yields and limit potential product degradation. Nevertheless, immunopurification performed with conventional antibodies often requests extreme elution conditions that can damage the purified product. Single-domain antibody fragments, such as VHHs or Nanobodies® (Nbs) have been used in affinity chromatography (AC), and are fairly stable in different elution conditions, and easy to produce, positioning them as a suitable tool in immunochromatography (e.g. Verheesen et al. 2003). VHH-based affinity columns also enable higher yields, as compared to longer antibody constructs and complete IgGs, probably because of the higher density at which they are bound to the matrix (Aliprandi et al., 2010). The suitability of VHHs for affinity purification is further acknowledged by a number of VHH-based affinity resins commercially developed and commonly applied, for example the Capture Select resins (http://www.captureselect.com), the Chromotek nanotrap for the purification of GFP-fused proteins from cell homogenates (Rothbauer et al., 2008; U.S. Pat. No. 10,125,166B2) among others (e.g. Pabst et al., 2016). Comparable to the EPEA tag, which is a C-terminal tag detected by the Nb-based CaptureSelect resin (U.S. Pat. No. 9,518,084B2; EP2576609B1), Nanotag™ Biotechnologies (Gotzke et al. 2019) developed a new Alpha peptide tag-based purification technology applying an ALPHA-specific Nb, combined with high affinity peptides for elution of the ALFA-tagged target protein bound to the Nb.

Frequently, single-domain antibody fragments, VHHs or Nanobodies are chemically attached to an insoluble matrix, and bound protein complexes are eluted under conditions that destroy non-covalent interactions between proteins. Instead, an enzyme that hydrolyzes a bond to allow detachment of the VHH (along with any bound protein complex) from the matrix was used by Pleiner et al. (2015, eLife; 4:e11349.), thereby eluting a bound protein complex including the VHH allowing further studies, such as structural analysis or physicochemical characterization. A drawback is that certain concentrations of enzyme, as well as specific conditions for optimal enzymatic activity are required within the sample. Moreover, the protease that is used can also harm the bound target protein and the column or matrix can only be used once. Methods combining Nanobody-based assets, such as in a competitive immunoassay have also been disclosed for use protein detection by ELISA (Caljon et al., 2015), wherein Nbs binding to distinct epitopes on a target were combined.

Alternatively, in sample displacement chromatography (SDC), the sample is introduced onto a column, and then displaced by a constant infusion of a displacer solution. Displacement chromatography of proteins and peptides is usually performed in ion-exchange mode, but hydrophobic interaction mode and affinity has also been used. The affinity of the displacer for the stationary phase must be higher than the affinity of any feed components. SDC integrated in small analytical columns for effective separation of microgram amounts of proteins from human plasma may be applied as sample preparation step for subsequent mass spectrometry (MS) analysis of separated proteins. Though, reversed-phase mode (RPC) is still the basic method for separation of target peptides after synthesis in order to remove trace impurities, and most commonly applied chromatography step for the separation of proteolytic digests of proteins prior to MS analysis. However, development of a rapid, shallow, reproducible, and cost-effective method for the efficient purification of proteins and peptides from complex mixtures is still a challenge. Although chromatographic supports and instruments have been further improved, there is still a need for the development of new, complementary methods for the separation of complex mixtures of proteins and peptides in both analytical and preparative scales. Affinity- or immune-displacement assays can be applied in mild conditions and have been described as an elegant antibody-based chromatography tool. For instance, Abdiche et al. (2017) have applied a ‘waterfall’ mechanism of monoclonal antibodies with adjacent or minimally overlapping epitopes for a target molecule, to displace and specifically elute the target. The drawback of using monoclonal antibodies for displacement is that they cross-block instead of displace each other if epitope diversity is too low, and a number of highly specific distinct antibodies are required, characterized by different association kinetics for non-overlapping or minimally-overlapping epitopes as to obtain optimal displacement.

The downscaling of straightforward, fast and easy protein purification for direct isolation of proteins or protein complexes from a complex mixture in conditions suited for analytical purposes such as structural biology, mass spectrometry analysis or proteomics is currently feasible using affinity purification, though not suitable for high-throughput purposes. Neither is there a possibility to apply generic binders for a particular binding site or epitope, making it laborious to determine distinct binders for each target, while in fact a more generic approach is desired. So, there is a need for straightforward high purity small scale purification methods that allow direct analytical tests on small amounts of target protein from complex mixtures.

### SUMMARY OF THE INVENTION

The present invention describes a new method of Nanobody-based displacement or competition-based exchange chromatography, wherein a pair of Nbs, competing for binding the target protein, with possibly the same or highly overlapping epitopes on a target, is used to purify the protein of interest (or ‘target protein’ as used interchangeably herein) from a complex mixture, and in a single step. The purification method allows to displace competing binders for a protein of interest, and is based on the finding that when using a Nanobody, or by extension an immunoglobulin single variable domain (ISVD) antigen-binding domain, as a displacer the displacement kinetics is different as compared to what is expected for conventional antibody antigen-binding domains. Furthermore, the other advantages of ISVD-based binders, such as their binding regions capable of binding conformational epitopes and deep clefts, their high stability, and easy manufacturability provide for the method as presented herein being suitable for high-throughput analytical affinity purification. Said method thereby yielding small amounts of highly pure protein bound to a high affinity Nb, which may be labelled, functionalized or used as a chaperone for structural or biochemical analysis. In Nanobody-exchange chromatography (shortened herein as NANEX), the protein of interest or target protein is captured by a first (immobilized) Nanobody, called Nanotrapper or trapper, followed by selective elution of the bound protein via binding to a second soluble Nanobody, called Nanostripper or stripper, which competes with the trapper for binding to the target protein, but has displacement kinetic properties favorable to establish an efficient displacement and elution. More specifically, the kinetics are determined for said ISVD-containing stripper by the dissociation rate constant, and require a slower dissociation rate (or lower k_{off}) and/or higher affinity (or lower K_{D}) and/or higher avidity as compared to the capturing agent or trapper. This process results in a quantitative and fast elution at physiological conditions of a very pure complex of the protein of interest bound to the stripper. Optionally, the Nanostripper may be functionalized as a chaperone, stabilizer, or antigen-binding chimeric protein known as a MegaBody™, or alternatively has a detectable label or properties facilitating subsequent analysis.

In a first aspect, the invention relates to a method for purification of a target protein comprising the steps of:
a) contacting a first protein binding agent specifically binding an epitope of a target protein with a sample containing said target protein,
b) mixing the sample with a second protein binding agent competing for binding the target protein when the first protein binding agent is present, so that the second protein binding agent replaces the first binding agent on the target protein, and releases the first binding agent from the target protein, and
c) eluting the mixture containing the second protein binding agent bound to the target protein,
   wherein at least the second protein binding agent comprises an immunoglobulin single variable domain (ISVD) or a functional variant thereof specifically binding the target protein, and wherein the rate constant of dissociation (or the k_{off} value) of the second protein binding agent is lower or identical, or the dissociation rate is slower or the same, as compared to the first protein binding agent. The second protein binding agent may compete through binding an epitope on the target protein that is the same or largely overlapping. Alternatively, the second binding agent may bind a different or minimally overlapping epitope, but allosterically and/or kinetically compete for binding the target protein, as driven by its dissociation constant rate. In a further embodiment, said second protein binding agent has higher affinity, i.e. a K_{D} value that is lower, for the epitope, as compared to the first protein binding agent. More specifically, the K_{D} value for the epitope of the target protein is in the low micromolar to nanomolar range for the first protein binding agent and in the low nanomolar to picomolar range for the second protein binding agent. Preferably, the relative affinity is defined as the K_{D} value of the first protein binding agent being at least 2 fold, or preferably at least 10-fold, 20-fold, or 100-fold higher as compared to the K_{D} value of the second protein binding agent.

In another embodiment the method as described herein comprises a washing step of the mixture of step a) prior to adding the second protein binding agent, to remove impurities and provide suitable buffer conditions.

Another embodiment discloses the method for purification of a target protein as described herein, further comprising the steps of: repeating, or altering steps a) to c), using a 3^{rd} and 4^{th} protein binding agent instead of, or in addition to the 1^{st} and 2^{nd} protein binding agents, respectively, wherein said 3^{rd} and 4^{th} binding agents specifically bind the same target protein of step a) to c), but the epitope being different from the epitope binding the 1^{st} and 2^{nd} binding agent. Said tandem-purification method specifically relates to the purification from complex samples to obtain a higher purity. Optionally a washing step may be included in said method as to remove unbound proteins or excess binding agents.

Furthermore, the method as disclosed herein uses a first and second binding agent competing for binding to a target protein present in the sample, wherein the binding agents specifically recognize an epitope on a tag of said target protein, preferably as part of a fusion protein, preferably wherein said tag may involve an affinity tag, an epitope tag, a reporter tag, or another synthetic and/or commercially available tag. Said tag of the target protein may be selected from the group of fluorescent proteins (such as green fluorescent protein (GFP), or mCherry), may be glutathione-S-transferase (GST), Small ubiquitin-like modifier (SUMO), SMT3, the C-terminal peptide EPEA, among others as listed further herein. Alternatively, the epitope of the target protein recognized by said protein binding agents comprises a specific epitope present on a native or on an endogenous protein, or a naturally displayed epitope of the protein as present in nature. Furthermore, the epitope may also be established by a post-translational modification (PTM) on the target protein, specifically bound by the protein binding agents recognizing said PTM. A further alternative relates to the method of the present invention, wherein the epitope of the target protein is defined by a specific binding site on the scaffold protein domain of a Mega Body™, i.e. an antigen-binding chimeric protein as defined in Steyaert et al. (WO2019/086548A1). In a preferred embodiment, said epitope is specific for the scaffold protein domain of the antigen-binding chimeric protein comprising HopQ- or Ygjk-derived scaffolds as disclosed in WO2019/086548A1.

Another embodiment relates to said method wherein the second protein binding agent comprises the first protein binding agent in a multivalent format, or in a multispecific format. The method as described herein comprises a second protein binding agent comprising an ISVD defined herein as a domain with 4 Framework regions (FR) and 3 complementary determining regions (CDR) according to the format of FR1-CDR1-FR2-CDR2-FR3-CDR3-FR4, which is sufficient for binding the target protein or antigen. Alternatively, a functional variant of an ISVD is meant herein, and relates to an ISVD-containing moiety that is capable of binding the antigen in a similar way as the ISVD. The method may further employ a first protein binding agent which also comprises an ISVD, specifically binding and competing with the second binding agent for the target epitope.

The method as disclosed herein further relates to competing first and second protein binding agents, wherein the dissociation rate of the second binding agent for target binding is slower as compared to dissociation rate of the first binding agent, and wherein the first binding agent constitutes a mutant ISVD as compared to the second ISVD-comprising protein binding agent, or vice versa, and wherein said first ISVD-comprising binding agent has a faster dissociation rate, and/or lower affinity (or higher K_{D}) as compared to the second ISVD-comprising binding agent, as cause by the alteration in its binding region or paratope.

An alternative embodiment provides for a method as described herein, wherein the first and second binding agent comprise the same ISVD binding moiety, which specifically binds the target protein with a k_{off} of minimally 0.0001 s⁻1 or higher.

A further embodiment relates to the method as presented herein, wherein the protein binding agent(s) comprise a functional moiety or a detectable label.

In specific embodiment, the first and/or second protein binding agent of the method described herein is in a functionalized format, i.e. has a format with a particular function besides binding the epitope. For example, said functionalized format may comprise an antigen-binding chimeric protein, in particular a MegaBody™, as disclosed in Steyaert et al. (WO2019/086548A1). Said Mega Body as referred to herein comprises an antigen-binding domain in the format of an ISVD functional variant D, specifically binding the epitope of the target protein via the ISVD, wherein said ISVD antigen-binding domain is rigidly fused to a scaffold protein domain. In a preferred embodiment said scaffold protein comprises or is derived from HopQ or Ygjk protein. Said MegaBody is known to provide for a function as a novel chaperone-type of binding agent for its improvement of cryo-EM structural analysis of the target protein.

An embodiment relates to said method as described herein wherein the first protein binding agent is immobilized on a surface, and the second protein binding agent is in solution, meaning that the second binding agent is soluble under suitable purification conditions. Preferably, said first protein binding agent surface is a resin, and suitable conditions are physiological conditions. Said resin or matrix may be suited for preparative purification or may be for analytical purification, the latter preferably with a volume as low as few microliters. An alternative aspect of the invention relates in fact to a chip or microcolumn comprising said first protein binding agent in immobilized form on a surface, and being setup for using said chip in the method as described herein, so in combination with a solution providing for the second protein binding agent as described in the method herein.

The method of the present invention provides for purification of a target protein or molecule from a sample, wherein said sample may be a biological sample, a complex mixture, a cellular sample, or an in vitro sample.

Another aspect relates to a kit comprising the first and second protein binding agent for use in the method as described herein. A further embodiment relates to the kit comprising the first and second protein binding agent according to the invention, wherein said first or second agent is present on a surface, matrix or resin, or wherein said kit comprises the microchip as described herein. More specifically, the kit may comprise a first and second protein binding agent competing for binding to a tag of a target protein, wherein said tag is selected from the group of tags containing GFP, mCherry, GST, SMT3, or EPEA, and wherein said agents comprise a sequence selected from the group of proteins as depicted in SEQ ID NO: 1-6, 18, or 19, 20, 21, 23, 24, 26, 27, or 28, or a sequence with at least 90% identity thereof, or any of said sequences without the His and/or EPEA tag, optionally comprising another (small) tag. In a specific embodiment said first and second protein binding agent of the kit comprise a different sequence selected from said group. In another specific embodiment, said first and second protein binding agent of the kit may comprise the same sequence selected from said group, wherein the K_{D} is equal or above 0.1 nM.

Another aspect relates to a protein complex comprising the second or fourth protein binding agent and the target protein as disclosed in the method for purification herein. Said target protein may in particular embodiments be selected from the group of GFP, mCherry, GST, SMT3, or EPEA. In one embodiment, said protein complex may be crystalline. The protein complex as defined herein may further comprise one or more additional proteins bound to the target protein. Said further complex provides for a use in identification or characterization of protein-protein complexes or interactions, which may be transient or conformation-specific. Finally said protein complexes as disclosed herein may be of use for structural analysis, structure-based drug design, drug discovery, mass-spectrometry analysis or alternative biochemical or physicochemical analyses.

Another alternative aspect described herein relates to a high-resolution three-dimensional structural representation at atomic resolution of the protein complex formed by the second (or fourth) protein binding agent and the target protein. For a crystalline complex as disclosed herein, one embodiment particularly relates to the crystal of GFP and a GFP-specific Nb characterized in that the crystal is in the space group P212121 with unit-cell parameters: a=74.497 Å±5%, b=103.450 Å±5%, c=209.774 Å±5%, α=90.00°, β=90.00°, [symbol] =90.00°. Said 3D structure or crystal provides for an embodiment disclosing a specific epitope of binding site for the protein binding agents specific for GFP as disclosed herein, said binding site consisting of a subset of atomic coordinates, wherein said binding site consists of the amino acid residues: PRO89, GLU90, GLU111, LYS113, PHE114, GLU115, GLY116 of the GFP protein as depicted in SEQ ID NO: 16.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIG. 1. Schematic representation of Na nobody exchange chromatography (NAN EX) principle.

1) A protein of interest (POI) is retained on beads coated with a specific Nanobody trapper (grey spheres) and 2) eluted using a Nanobody stripper (hatched spheres) that binds to an overlapping epitope on the POI.

FIG. 2. Affinity purification of a GFP-tag spiked in a bacterial lysate using CA15816 as an immobilized trapper on HiTrap NHS-activated Sepharose HP columns and eluted with CA12760 as a stripper.

NANEX purification of GFP using CA15816 a medium-affinity trapper immobilized on a HiTrap NHS-activated Sepharose HP column (1 mL) and eluted using CA12760 a high-affinity stripper. 2 mg of purified GFP was spiked into a bacterial lysate. The CA15816-column was washed twice with 10 CV of buffer (100 mM Hepes pH7.5, 150 mM NaCl) followed by the injection of the stripper. Left panel: brief description of the procedure. Middle panel: elution chromatogram showing absorbance at 280 nm and 488 nm. The high peak on the left eluted upon injection of CA12760 stripper. The right peak eluted upon regeneration with 200 mM glycine at pH 2.3. Right panel: SDS-PAGE of the different purification steps, molecular weight marker (PageRuler™ Prestained Protein Ladder from ThermoFisher cat. 26616).

FIG. 3. X-ray structure of the GFP⋅CA12760 protein complex and description of the epitope.

Left: Crystal structure of CA12760 (ribbon representation) in complex with GFP (surface representation). Middle: Surface representation of GFP. Residues composing the epitope of CA12760 on GFP are colored in dark grey and labeled. Right: table summarizing the residues that compose the CA12760 binding epitope on GFP.

FIG. 4. View on the CA12760⋅GFP interface to highlight the three residues on CA12760 Nb that were selected for mutagenesis (Thr54, Val55, Phe103) to design lower affinity trappers (CA15818, CA15816, CA15861).

GFP is represented in surface mode. CA12760 is represented in ribbon mode. Thr54, Val55, and Phe103 are represented as sticks.

FIGS. 5A and 5B. Kinetic characterization of the interaction of GFP with stripper CA12760 and the trappers derived thereof by mutagenesis (CA15818, CA15816, CA15861).

Real-time kinetic analysis of the binding of GFP to Nanobody CA12760 (SEQ ID NO: 1), CA15818 (SEQ ID NO: 2), CA15816 (SEQ ID NO: 3), and CA15861 (SEQ ID NO: 4), respectively. Streptavidin-coated Octet® biosensors were used to capture biotinylated Nanobodies (1 μg/mL). Binding and dissociation isotherms at several GFP concentrations (1 nM to 5 μM range) were analyzed on an OctetRed (molecular devices). All assays were performed in Hepes 25 mM pH7.5, NaCl 150 mM supplemented with BSA 0.1% and Tween²⁰ 0.005% at room temperature.

FIG. 6. GFP target proteins trapped with the high affinity Nanobody (CA12760) are poorly eluted with lower affinity Nanobodies (CA15818, CA15816, CA15861) that bind the same epitope.

NANEX purification of GFP using CA12760 (high-affinity trapper) as an immobilized trapper on NHS-Activated agarose beads and CA12760, CA15818, CA15816, CA15861 as strippers. 50 μl of CA12760 agarose beads were mixed in an Eppendorf tube with 200 μg GFP, washed and incubated with 53 μM (800 μg/mL) of strippers in a final volume of 1 mL (100 mM Hepes pH 7.5, 150 mM NaCl). GFP elution was monitored by spinning down the beads at 5 different time points (0, 15, 30, 60, 120 minutes) and measuring the absorbance of the supernatant at 488 nm.

FIG. 7. GFP target proteins trapped with high affinity Nanobody (CA15818) are poorly eluted with lower affinity Nanobodies (CA15818, CA15816, CA15861) that bind the same epitope.

NANEX purification of GFP using CA15818 (medium affinity trapper) as an immobilized trapper on NHS-Activated agarose beads and CA12760, CA15818, CA15816, CA15861 as strippers. 50 μl of CA12760 agarose beads were mixed in an Eppendorf tube with 200 μg GFP, washed and incubated with 53 μM (800 μg/mL) of strippers in a final volume of 1 mL (100 mM Hepes pH 7.5, 150 mM NaCl). GFP elution was monitored by spinning down the beads at 5 different time points (0, 15, 30, 60, 120 minutes) and measuring the absorbance of the supernatant at 488 nm.

FIG. 8. GFP target proteins trapped with a medium affinity Nanobody (CA15816) are poorly eluted with lower affinity Nanobody (CA15861) that binds the same epitope, but elute fast and quantitatively with high affinity Nanobodies (CA12760, CA15818, CA15816) that bind the same epitope.

NANEX purification of GFP using CA15816 (medium affinity trapper) as an immobilized trapper on NHS-Activated agarose beads and CA12760, CA15818, CA15816, CA15861 as strippers. 50 μl of CA12760 agarose beads were mixed in an Eppendorf tube with 200 μg GFP, washed and incubated with 53 μM (800 μg/mL) of strippers in a final volume of 1 mL (100 mM Hepes pH 7.5, 150 mM NaCl). GFP elution was monitored by spinning down the beads at 5 different time points (0, 15, 30, 60, 120 minutes) and measuring the absorbance of the supernatant at 488 nm.

FIG. 9. GFP target proteins trapped with low affinity Nanobody (CA15861) are eluted fast and quantitatively with high affinity Nanobodies (CA12760, CA15818, CA15816) that bind the same epitope but weakly capture the target protein.

NANEX purification of GFP using CA15861 (low-affinity trapper) as an immobilized trapper on NHS-Activated agarose beads and CA12760, CA15818, CA15816, CA15861 as strippers. 50 μl of CA12760 agarose beads were mixed in an Eppendorf tube with 200 μg GFP, washed and incubated with 53 μM (800 μg/mL) of strippers in a final volume of 1 mL (100 mM Hepes pH 7.5, 150 mM NaCl). GFP elution was monitored by spinning down the beads at 5 different time points (0, 15, 30, 60, 120 minutes) and measuring the absorbance of the supernatant at 488 nm.

FIGS. 10A-10C. GFP target proteins trapped with high affinity Nanobodies (CA12760) are poorly eluted with low affinity Nanobodies (CA15818, CA15816, CA15861) that bind the same epitope in NANEX.

For the purification of GFP by NANEX, CA12760 (high-affinity trapper) was immobilized as a trapper on a HiTrap NHS-activated Sepharose HP column (1 mL). For each experiment, 2 mg of purified GFP was injected on this NANEX column. The loaded CA12760-column was washed twice with 10 CV of buffer (100 mM Hepes pH 7.5, 150 mM NaCl) followed by the injection of strippers CA12760, CA15818, CA15816, CA15861, per column, respectively. The elution of GFP by these different strippers was monitored by following the absorbance at 280 nm and 488 nm. The high peak on the left eluted upon injection of the respective stripper (CA12760, CA15818, CA15816, CA15861). The right peak eluted upon regeneration of the columns with 200 mM glycine at pH 2.3. Right panel SDS-PAGE of the different purification steps, molecular weight marker (PageRuler™ Prestained Protein Ladder from ThermoFisher cat. 26616).

FIGS. 11A-11C. GFP target proteins trapped with a medium affinity Nanobodies (CA15816) are poorly eluted with low affinity Nanobodies (CA15861) that bind the same epitope, but elute fast and quantitatively with high affinity Nanobodies (CA12760, CA15818, CA15816) that bind the same epitope in NANEX.

For the purification of GFP by NANEX, CA15816 (medium-affinity trapper) was immobilized on a HiTrap NHS-activated Sepharose HP column (1 mL). For each experiment, 2 mg of purified GFP was injected on this NANEX column. The loaded CA15816-column was washed twice with 10 CV of buffer (100 mM Hepes pH 7.5, 150 mM NaCl) followed by the injection of the respective stripper (CA12760, CA15818, CA15816, CA15861). The elution of GFP by these different strippers was monitored by following the absorbance at 280 nm and 488 nm. The high peak on the left eluted upon injection of the strippers (CA12760, CA15818, CA15816, CA15861). The right peak eluted upon regeneration of the column with 200 mM glycine at pH 2.3. Right panel SDS-PAGE of the different purification steps, molecular weight marker (PageRuler™ Prestained Protein Ladder).

FIGS. 12A-12C. GFP target proteins trapped with low affinity Nanobody (CA15861) are eluted fast and quantitatively with high affinity Nanobodies (CA12760, CA15818, CA15816) that bind the same epitope in NANEX, but weakly capture the target protein.

For the purification of GFP by Na nobody exchange chromatography, CA15861 (low-affinity trapper) was immobilized as a trapper on a HiTrap NHS-activated Sepharose HP column (1 mL). For each experiment, 2 mg of purified GFP was injected on this NANEX column. The loaded CA15861-column was washed twice with 10 CV of buffer (100 mM Hepes pH 7.5, 150 mM NaCl) followed by the injection of respective stripper (CA12760, CA15818, CA15816, CA15861). The elution of GFP by these different strippers was monitored by following the absorbance at 280 nm and 488 nm. The high peak on the left eluted upon injection of the strippers (CA12760, CA15818, CA15816, CA15861). The right peak eluted upon regeneration of the column with 20 0 mM glycine at pH 2.3. Right panel SDS-PAGE of the different purification steps, molecular weight marker (PageRuler™ Prestained Protein Ladder).

FIG. 13. NANEX purification of GFP using CA15816 a medium-affinity trapper and CA15621, a functionalized Nanobody (i.e. Mega Body Mb_{CA12760}^{cHopQ}), as a high-affinity stripper.

For the purification of GFP by NANEX, CA15816 (medium-affinity trapper) was immobilized as a trapper on a HiTrap NHS-activated Sepharose HP column (1 mL). 2 mg of purified GFP was injected on this NANEX column. The loaded CA15816-column was washed twice with 10 CV of buffer (100 mM Hepes pH 7.5, 150 mM NaCl) followed by the injection of the stripper CA15621 a functionalized Nanobody (Mega Body Mb_{CA12760}^{cHopQ}). The elution of GFP by this stripper was monitored by following the absorbance at 280 nm and 488 nm. The high peak on the left eluted upon injection of the stripper (CA15621). The right peak eluted upon regeneration with 200 mM glycine at pH 2.3. Right panel SDS-PAGE of the different purification steps, molecular weight marker (PageRuler™ Prestained Protein Ladder).

FIG. 14. NANEX purification of GFP using CA15816 a medium-affinity trapper and CA15616, a functionalized Nanobody (i.e. Mega Body Mb_{CA12760}^{YgjK}), as a high-affinity stripper.

For the purification of GFP by NANEX chromatography, CA15816 (medium-affinity trapper) was immobilized as a trapper on a HiTrap NHS-activated Sepharose HP column (1 mL). 2 mg of purified GFP was injected on this NANEX column. The loaded CA15816-column was washed twice with 10 CV of buffer (100 mM Hepes pH 7.5, 150 mM NaCl) followed by the injection of the stripper CA15616 a functionalized Nanobody (MegaBody Mb_{CA12760}^{YgjK}). The elution of GFP by this stripper was monitored by following the absorbance at 280 nm and 488 nm. The high peak on the left eluted upon injection of the stripper (CA15616). The right peak eluted upon regeneration with 200 mM glycine at pH 2.3. Right panel SDS-PAGE of the different purification steps, molecular weight marker (PageRuler™ Prestained Protein Ladder).

FIG. 15. NANEX purification of GFP using CA15816 a medium-affinity trapper and CA12760 as a high-affinity stripper on a 75 μL microcolumn.

For the purification of GFP by NANEX, CA15816 (medium-affinity trapper) was immobilized as a trapper on NHS-Activated agarose beads to prepare a custom-made micro-column. 0.1 mg of purified GFP was injected on this NANEX microcolumn. The loaded CA15816-microcolumn was washed twice with 5 mL of buffer (100 mM Hepes pH 7.5, 150 mM NaCl) followed by the injection of the stripper CA12760 (high-affinity). The elution of GFP by this stripper was monitored by following the absorbance at 280 nm and 488 nm. The high peak on the left eluted upon injection of the stripper (CA12760). The right peak eluted upon regeneration with 200 mM glycine at pH 2.3. Right panel SDS-PAGE of the different purification steps, molecular weight marker (PageRuler™ Prestained Protein Ladder).

FIG. 16. NANEX purification of EPEA-tagged GFP (GFP-EPEA) protein using an EPEA-specific Nanobody (CA4375) as a medium-affinity trapper and CA4375 as a medium-affinity stripper.

For the purification of EPEA-tagged GFP (GFP-EPEA) by NANEX, CA4375 (medium-affinity trapper) was immobilized as a trapper on a HiTrap NHS-activated Sepharose HP column (1 mL). 1 mg of purified EPEA-tagged GFP (GFP-EPEA) protein was injected on this NANEX column. The loaded CA4375-column was washed twice with 10 CV of buffer (100 mM Hepes pH 7.5, 150 mM NaCl) followed by the injection of the stripper CA4375 (medium-affinity stripper). The elution of EPEA-tagged GFP (GFP-EPEA) by this stripper was monitored by following the absorbance at 280 nm and 488 nm. The high peak on the left eluted upon injection of the stripper (CA4375). The right peak eluted upon regeneration with 200 mM glycine at pH 2.3. Right panel SDS-PAGE of the different purification steps, molecular weight marker (PageRuler™ Prestained Protein Ladder).

FIG. 17. NANEX-column purification of EPEA-tagged GFP (GFP-EPEA) protein using an EPEA-specific Nanobody (CA4375) as a medium-affinity trapper and a bivalent CA4375 as a high-affinity stripper.

For the purification of EPEA-tagged GFP (GFP-EPEA) protein by Nanobody exchange chromatography, CA4375 (medium-affinity trapper) was immobilized as a trapper on a HiTrap NHS-activated Sepharose HP column (1 mL). 1 mg of purified EPEA-tagged GFP (GFP-EPEA) protein was spiked into a bacterial lysate and injected on this NANEX column. The loaded CA4375-column was washed twice with 10 CV of buffer (100 mM Hepes pH 7.5, 150 mM NaCl) followed by the injection of the stripper a bivalent CA4375 (high-affinity stripper). The elution of EPEA-tagged GFP (GFP-EPEA) protein by this stripper was monitored by following the absorbance at 280 nm and 488 nm. The high peak on the left eluted upon injection of the stripper (bivalent CA4375). The right peak eluted upon regeneration with 200 mM glycine at pH 2.3. Right panel SDS-PAGE of the different purification steps, molecular weight marker (PageRuler™ Prestained Protein Ladder).

FIG. 18. NANEX-column purification of recombinant human Synaptojanin using a Synaptojanin-specific Nanobody (CA13016) as a medium-affinity trapper and CA13080 as a high-affinity stripper.

For the purification of recombinant human Synaptojanin by Nanobody exchange chromatography, CA13016 (medium-affinity trapper) was immobilized as a trapper on a HiTrap NHS-activated Sepharose HP column (1 mL). 10 mL of bacterial lysate containing overexpressed recombinant human Synaptojanin was injected on this NANEX column. The loaded CA13016-column was washed twice with 10 CV of buffer (100 mM Hepes pH 7.5, 150 mM NaCl) followed by the injection of the stripper CA13080 (high-affinity stripper). The elution of recombinant human Synaptojanin by this stripper was monitored by following the absorbance at 280 nm. The high peak on the left eluted upon injection of the stripper (CA13080). The right peak eluted upon addition of 200 mM glycine at pH 2.3. Right panel SDS-PAGE of the different purification steps, molecular weight marker (PageRuler™ Prestained Protein Ladder).

FIG. 19. NANEX-column purification of recombinant human coagulation factor IXa using a factor IXa-specific Nanobody (CA11138) as a medium-affinity trapper, and CA10304 as a high-affinity stripper.

For the purification of recombinant human coagulation factor IXa by NANEX, CA11138 (medium-affinity trapper) was immobilized as a trapper on a HiTrap NHS-activated Sepharose HP column (1 mL). 0.4 mg of purified recombinant human coagulation factor IXa fluorescently labelled with Dylight-647 was injected on this NANEX column. The loaded CA11138-column was washed twice with 10 CV of buffer (100 mM Hepes pH 7.5, 150 mM NaCl) followed by the injection of the stripper CA10304 (high-affinity stripper). The elution of recombinant human coagulation factor IXa by this stripper was monitored by following the absorbance at 280 nm and 650 nm. The high peak on the left eluted upon injection of the stripper (CA10304). The right peak eluted upon addition of 200 mM glycine at pH 2.3. Right panel SDS-PAGE of the different purification steps, molecular weight marker (PageRuler™ Prestained Protein Ladder).

FIG. 20. NANEX-column purification of recombinant human coagulation factor IXa⋅CA10304 using factor IXa-specific Nanobody (CA10502) as a medium-affinity trapper, and CA10309 as a high-affinity stripper.

For the purification of recombinant human coagulation factor IXa⋅CA10304 complex by NANEX, CA10502 (medium-affinity trapper) was immobilized as a trapper on a HiTrap NHS-activated Sepharose HP column (1 mL). Purified recombinant human coagulation factor IXa⋅CA10304 from Example 14 was injected on this NANEX column. The loaded CA10502-column was washed twice with 10 CV of buffer (100 mM Hepes pH 7.5, 150 mM NaCl) followed by the injection of the stripper CA10309 (high-affinity stripper). The elution of recombinant human coagulation factor IXa by this stripper was monitored by following the absorbance at 280 nm and 650 nm. The high peak on the left eluted upon injection of the stripper (CA10309). The right peak eluted upon addition of 200 mM glycine at pH 2.3. Right panel SDS-PAGE of the different purification steps, molecular weight marker (PageRuler™ Prestained Protein Ladder).

FIG. 21. Schematic representation of a Tandem Nanobody exchange chromatography (Tandem-NANEX).

In this example, two chromatographic columns containing different affinity matrices are connected. Tandem-NANEX can also be performed by mixing the different affinity matrices in a single column. This scheme shows the Tandem Nanobody exchange chromatography (tandem-NANEX) principle which uses two Nanobody pairs that pairwise compete for two different epitopes.

1) As a first purification step the protein of interest (POI) is retained on beads coated with a specific Nanobody trapper1 (beads1 coupled to grey spheres). 2) After washing the target is eluted from eluted using a Nanobody stripper1 (tilted hatched spheres) that binds to an overlapping epitope of trapper 1 on the POI. As a result, the POI⋅Stripper1 complex is retained on beads that are coated with trapper2 that binds another epitope (black spheres) 3) the POI⋅Stripper1 complex can be eluted using a stripper that overlaps with tarpper2 (stripper 2, vertical hatched spheres) to recover POI⋅Stripper1⋅Stripper2 as a highly purified ternary complex.

FIG. 22. Tandem-NANEX purification of recombinant human coagulation factor IXa using a factor IXa-specific Nanobodies CA11138 as a first trapper and CA10304 as a first stripper followed by CA10502 as a second trapper and CA14208, a functionalized Nanobody (MegaBody Mb_{CA10309}^{YgjK}), as a second stripper.

For the purification of recombinant human coagulation factor IXa by Tandem-NANEX, a first NANEX column where CA11138 (medium-affinity trapper) was immobilized as a trapper1 on a HiTrap NHS-activated Sepharose HP column (1 mL) was connected to a second NANEX column consisting of CA10502 (medium-affinity trapper) immobilized as a trapper2 on a HiTrap NHS-activated Sepharose HP column (1 mL). 0.4 mg of purified recombinant human coagulation factor IXa fluorescently labelled with Dylight-647 was injected on both NANEX columns that were washed twice with 10 CV of buffer (100 mM Hepes pH 7.5, 150 mM NaCl) then connected to on an Akta-Pure (GE) FLPC system. Injection of the stripper1 CA10304 (high-affinity stripper) was followed by a washing step of 5 mL of the same buffer before the injection of the stripper2 CA14208 (high-affinity stripper). The elution of recombinant human coagulation factor IXa by both strippers was monitored by following the absorbance at 280 nm and 650 nm. The peak on the left eluted upon injection of the stripper1 (CA10304). The right peak eluted upon injection of the stripper2 (CA14208). Right panel SDS-PAGE of the different purification steps, molecular weight marker (PageRuler™ Prestained Protein Ladder from ThermoFisher cat. 26616).

FIG. 23: NANEX purification of the yeast 60S ribosomal subunit that contains the RPP1A-GFP fusion protein from a yeast extract using CA15816 as a trapper and CA12760 as a high-affinity stripper.

For the purification of GFP-RPP1A protein by NANEX, CA15816 (medium-affinity trapper) was immobilized as a trapper on a HiTrap NHS-activated Sepharose HP column (1 mL). 20 mL of clarified Yeast lysate was injected on this NANEX column. The loaded CA15816-column was washed twice with 10 CV of buffer (100 mM Hepes pH 7.5, 150 mM NaCl) followed by the injection of the high affinity stripper (CA12760). Elution of GFP-RPP1A protein by this stripper was monitored by following the absorbance at 280 nm and 488 nm. The high peak on the left eluted upon injection of the stripper (CA12760). The right peak eluted upon addition of 200 mM glycine at pH 2.3. Right panel SDS-PAGE of the different fractions of the main elution peak, molecular weight marker (PageRuler™ Prestained Protein Ladder).

FIG. 24. Visualization of the yeast 60S ribosomal subunit containing the GFP-tagged RPP1A ribosomal protein purified by NANEX from a yeast extract by negative stain electron microscopy.

For staining, 3 μl of the major eluting peak (fraction 6, 0.1 mg/mL protein concentration) from NANEX on a lysate of yeast clone GFP+35: G8 was applied for 30 seconds onto a glow-discharged grid and washed in uranyl acetate (2% w/v) for 30 s prior to drying. Images were taken on a Jeol1400 microscope with a 50× magnification.

FIGS. 25A-25C. X-ray structure of the GFPCA16047 proteinNanobody complex and description of the epitope.

FIG. 25A: Crystal structure of Nanobody CA16047 (ribbon representation) in complex with GFP (surface representation). FIG. 25B: Surface representation of GFP. Residues composing the epitope of CA16047 on GFP are colored in dark grey and labeled. FIG. 25C: table summarizing the residues that compose the CA16047 binding epitope on GFP.

FIG. 26. View on the GFP⋅CA16047 interface to highlight Tyr119 in CDR3 of Nanobody CA16047 that was selected for mutagenesis to design a lower affinity trapper (CA16695).

GFP is represented in surface mode. CA16047 is represented in ribbon mode. Tyr119 is represented as sticks.

FIG. 27. Kinetic characterization of the interaction of GFP with stripper CA16047 and the trapper derived thereof by mutagenesis (CA16695).

Real-time kinetic analysis of the binding and the dissociation of GFP to Nanobody CA16047 (SEQ ID NO: 18) and CA16695 (SEQ ID NO: 19). Streptavidin-coated Octet® biosensors were used to capture biotinylated Nanobodies (1 μg/mL). Binding and dissociation isotherms at several GFP concentrations (8 nM to 500 nM range) were analyzed on an OctetRed (molecular devices). All assays were performed in Hepes 25 mM pH7.5, NaCl 150 mM supplemented with BSA 0.1% and Tween²⁰ 0.005% at room temperature.

FIGS. 28A and 28B. NANEX purification of GFP using CA16695 a medium-affinity trapper and CA16047, as a high-affinity stripper.

For the purification of GFP by NANEX, CA16695 (medium-affinity trapper) was immobilized as a trapper on a HiTrap NHS-activated Sepharose HP column (1 mL). 2 mg of purified GFP was injected on this NANEX column. The loaded CA16695-column was washed twice with 10 CVs of buffer (100 mM Hepes pH 7.5, 150 mM NaCl) followed by the injection of 1 mg of the stripper CA16047. FIG. 28A) The elution of GFP by this stripper was monitored by following the absorbance at 280 nm and 488 nm. The high peak on the left eluted upon injection of the stripper (CA16047). The right peak eluted upon regeneration with 200 mM glycine at pH 2.3. FIG. 28B) SDS-PAGE of the different purification fractions, molecular weight marker (PageRuler™ Prestained Protein Ladder).

FIGS. 29A-29C. X-ray structure of the GST⋅CA16239 protein⋅Nanobody complex and description of the epitope.

FIG. 29A: Crystal structure of CA16239 (ribbon representation) in complex with GST (surface representation). FIG. 29B: Surface representation of GST. Residues composing the epitope of CA16239 on GST are colored in dark grey and labeled. FIG. 29C: table summarizing the residues that compose the CA16239 binding epitope on GST.

FIG. 30. View on GST⋅CA16239 interface to highlight residue Tyr109 in CDR3 of Nanobody CA16239 that was selected for mutagenesis to design a lower affinity trapper (CA16695).

GST is represented in surface mode. CA16239 is represented in ribbon mode. Tyr109 is represented as sticks.

FIG. 31. Kinetic characterization of the interaction of GST with stripper CA16239 and the trapper derived thereof by mutagenesis (CA16240).

Real-time kinetic analysis of the binding and the dissociation of GST to Nanobody CA16239 (SEQ ID NO: 20) and CA16240 (SEQ ID NO: 21). Streptavidin-coated Octet® biosensors were used to capture biotinylated Nanobodies (1 μg/mL). Binding and dissociation isotherms at several GST concentrations (63 nM to 1667 nM range) were analyzed on an OctetRed (molecular devices). All assays were performed in Hepes 25 mM pH7.5, NaCl 150 mM supplemented with BSA 0.1% and Tween²⁰ 0.005% at room temperature.

FIGS. 32A and 32B. NANEX purification of GST using CA16240 a medium-affinity trapper and CA16239, as a high-affinity stripper.

For the purification of GST by NANEX, 4 mg of CA16240 (medium-affinity trapper) was immobilized as a trapper on a HiTrap NHS-activated Sepharose HP column (1 mL). 2 mg of purified GST was injected on this NANEX column. The loaded CA16240-column was washed twice with 10 CVs of buffer (100 mM Hepes pH 7.5, 150 mM NaCl) followed by the injection of 2 mg of the stripper CA16239. FIG. 32A) The elution of GST by this stripper was monitored by following the absorbance at 280 nm. The high peak on the left eluted upon injection of the stripper (CA16239). The right peak eluted upon regeneration with 200 mM glycine at pH 2.3. FIG. 32B) SDS-PAGE of the different purification fractions, molecular weight marker (PageRuler™ Prestained Protein Ladder).

FIGS. 33A-33C. X-ray structure of the SMT3⋅CA15839 protein⋅Nanobody complex and description of the epitope.

FIG. 33A: Crystal structure of CA15839 (ribbon representation) in complex with SMT3 (surface representation). FIG. 33B: Surface representation of SMT3. Residues composing the epitope of CA15839 on SMT3 are colored in dark grey and labeled. FIG. 33C: table summarizing the residues that compose the CA15839 binding epitope on SMT3.

FIG. 34. View on the SMT3⋅CA15839 interface to highlight Asp50 on the surface of Nanobody CA15839 that was selected for mutagenesis to design a lower affinity trapper (CA16687).

SMT3 is represented in surface mode. CA15839 is represented in ribbon mode. Asp50 is represented as sticks.

FIG. 35. Kinetic characterization of the interaction of SMT3 with stripper CA15839 and the trapper derived thereof by mutagenesis (CA16687).

Real-time kinetic analysis of the binding and the dissociation of SMT3 to Nanobody CA15839 (SEQ ID NO: 23) and CA16687 (SEQ ID NO: 24). Streptavidin-coated Octet® biosensors were used to capture biotinylated Nanobodies (1 μg/mL). Binding and dissociation isotherms at several SMT3 concentrations (10 nM to 500 nM range) were analyzed on an OctetRed (molecular devices). All assays were performed in Hepes 25 mM pH7.5, NaCl 150 mM supplemented with BSA 0.1% and Tween²⁰ 0.005% at room temperature.

FIGS. 36A and 36B. NANEX purification of SMT3 using CA16687 a medium-affinity trapper and CA15839, as a high-affinity stripper.

For the purification of SMT3 by NANEX, 1 mg of purified CA16687 (medium-affinity trapper) was immobilized as a trapper on a HiTrap NHS-activated Sepharose HP column (1 mL). 2 mg of purified SMT3 was injected on this NANEX column. The loaded CA16687-column was washed twice with 10 CV of buffer (100 mM Hepes pH 7.5, 150 mM NaCl) followed by the injection of 2 mg of the stripper CA15839. FIG. 36A) The elution of SMT3 by this stripper was monitored by following the absorbance at 280 nm. The high peak on the left eluted upon injection of the stripper (CA15839). The right peak eluted upon regeneration with 200 mM glycine at pH 2.3. FIG. 36B) SDS-PAGE of the different purification fractions, molecular weight marker (PageRuler™ Prestained Protein Ladder).

FIGS. 37A and 37B. Epitope mapping of Nbs specific for mCherry by BLI using immobilized CA17302 on the biosensor.

FIG. 37A: Outline of the epitope mapping experiment. Streptavidin-coated Octet® biosensors where used to capture biotinylated CA17302 (1 μg/mL) (highest affinity trapper discovered against mCherry). Unbound biotinylated CA17302 are washed off from biosensor by two washing steps (30 seconds in buffer), followed by incubation with 100 nM mCherry, preincubated with 500 nM of the different Nbs to be tested. Association and dissociation rates are determined for 300 seconds and 600 seconds, respectively. FIG. 37B: Binding and dissociation isotherms for the positive and negative controls and the different Nbs tested, analyzed on an OctetRed (molecular devices). The complex formed between CA17302 and mCherry does not bind to the immobilized CA16964, indicating that these Nanobodies bind to an overlapping epitope. All assays were performed in Hepes 25 mM pH7.5, NaCl 150 mM supplemented with BSA 0.1% and Tween²⁰ 0.005% at room temperature.

FIG. 38. Kinetic characterization of the interaction of mCherry with stripper CA17302 and the trapper CA16964.

Real-time kinetic analysis of the binding of mCherry to Nanobody CA17302 (SEQ ID NO: 27) and CA16964 (SEQ ID NO: 26). Streptavidin-coated Octet® biosensors were used to capture biotinylated Nanobodies (1 μg/mL). Binding and dissociation isotherms at several mCherry concentrations (8.23 nM to 222 nM range) were analyzed on an OctetRed (molecular devices). All assays were performed in Hepes 25 mM pH7.5, NaCl 150 mM supplemented with BSA 0.1% and Tween²⁰ 0.005% at room temperature.

FIGS. 39A and 39B. Affinity purification of FmIH-lectin-mCherry-his using CA16964 as an immobilized trapper on HiTrap NHS-activated Sepharose HP columns and eluted with CA17302 as a stripper.

For the purification of mCherry by NANEX, 1 mg of CA16964 (medium-affinity trapper) was immobilized as a trapper on a HiTrap NHS-activated Sepharose HP column (1 mL). Recombinant FmIH-lectin-mCherry-his was expressed in BL21 expression strain by over-night induction at 28° C. using 1 mM IPGT. A 2 L bacterial pellet was lysed in 50 mL of resuspension buffer (25 mM HEPES pH 7.5, 150 mM NaCl) and clarified by centrifugation and filtering. This bacterial lysate was then manually injected on the NANEX column. The loaded CA16964-column was manually washed twice with 10 CV of buffer (100 mM Hepes pH 7.5, 150 mM NaCl) prior collecting the column to an Akta Pure, then followed by the injection of 1 mg of the stripper CA17302. FIG. 39A) The elution of FmIH-lectin-mCherry-his by this stripper was monitored by following the absorbance at 280 nm. The high peak on the left eluted upon injection of the stripper (CA17302). The right peak eluted upon regeneration with 200 mM glycine at pH 2.3. FIG. 39B) SDS-PAGE of the different purification fractions, molecular weight marker (PageRuler™ Prestained Protein Ladder). Asterisk (*), FmIH_lectin_mCherry_his protein.

FIG. 40. Kinetic characterization of the interaction of mCherry with stripper CA17302 and the trapper CA17341.

Real-time kinetic analysis of the binding of mCherry to Nanobody CA17302 (SEQ ID NO: 27) and CA17341 (SEQ ID NO: 28). Streptavidin-coated Octet® biosensors were used to capture biotinylated Nanobodies (1 μg/mL). Binding and dissociation isotherms at several mCherry concentrations (8.23 nM to 222 nM range) were analyzed on an OctetRed (molecular devices). All assays were performed in Hepes 25 mM pH7.5, NaCl 150 mM supplemented with BSA 0.1% and Tween²⁰ 0.005% at room temperature.

FIGS. 41A and 41B. Affinity purification of FmIH-lectin-mCherry-his using CA17341 as an immobilized trapper on HiTrap NHS-activated Sepharose HP columns and eluted with CA17302 as a stripper.

For the purification of mCherry by NANEX, 1 mg of CA17341 (medium-affinity trapper) was immobilized as a trapper on a HiTrap NHS-activated Sepharose HP column (1 mL). Recombinant FmIH-lectin-mCherry-his was expressed in BL21 expression strain by over-night induction at 28° C. using 1 mM IPGT. A 2 L bacterial pellet was lysed in 50 mL of resuspension buffer (25 mM HEPES pH 7.5, 150 mM NaCl) and clarified by centrifugation and filtering. This bacterial lysate was then manually injected on the NANEX column. The loaded CA17341-column was manually washed twice with 10 CV of buffer (100 mM Hepes pH 7.5, 150 mM NaCl) prior collecting the column to an Akta Pure, then followed by the injection of 1 mg of the stripper CA17302. FIG. 41A) The elution of FmIH-lectin-mCherry-his by this stripper was monitored by following the absorbance at 280 nm (protein absorbance) and 585 nm (mCherry absorbance). The high peak on the left eluted upon injection of the stripper (CA17302). The right peak eluted upon regeneration with 200 mM glycine at pH 2.3. FIG. 41B) SDS-PAGE of the different purification fractions, molecular weight marker (PageRuler™ Prestained Protein Ladder). Asterisk (*), FmIH_lectin_mCherry_his protein

FIGS. 42A-42C: NANEX purification of native human coagulation factor IX from human blood serum using a factor IX-specific Nanobody (CA11143) as a medium-affinity trapper, and MegaBody CA16383, as a stripper.

For the purification of native human coagulation factor IX by NANEX, CA11143 (medium-affinity trapper) was immobilized as a trapper on a HiTrap NHS-activated Sepharose HP column (1 mL). 30 mL of human recovered plasma treated with ACD anticoagulant was loaded on this NANEX column by recirculation for 120 minutes. The loaded CA11143-column was washed with 15 CV of buffer (20 mM Hepes, pH 8.0, 150 mM NaCl, 5 mM CaCl₂) followed by the injection of MegaBody CA16383. FIG. 42A) The elution of native human coagulation factor IX by this stripper was monitored by following the absorbance at 280 nm. The high peak on the left eluted upon injection of the stripper (CA16383). The right peak eluted upon addition of 200 mM glycine at pH 2.3. FIG. 42B) SDS-PAGE of representative fractions of the purification and FIG. 42C) the western blot of these fractions. MW, molecular weight marker (PageRuler™ Prestained Protein Ladder); and asterisk (*), commercial native human coagulation factor IX (used as control).

FIGS. 43A-43C. NANEX-purification of native human coagulation factor IX using MegaBody CA16388, as a medium-affinity trapper, and MegaBody CA16383 as a stripper.

For the purification of native human coagulation factor IX by NANEX, MegaBody CA16388 was immobilized as a medium-affinity trapper on a HiTrap NHS-activated Sepharose HP column (1 mL). 30 mL of human recovered plasma treated with ACD anticoagulant was loaded on this NANEX column by recirculation for 60 minutes. The loaded CA16388-column was washed with 15 CV of buffer (20 mM Hepes, pH 8.0, 150 mM NaCl, 5 mM CaCl₂) followed by the injection of the stripper Mega Body CA16383.

FIG. 43A) The elution of native human coagulation factor IX by this stripper was monitored by following the absorbance at 280 nm. The high peak on the left eluted upon injection of the stripper (CA16383). The right peak eluted upon addition of 200 mM glycine at pH 2.3. FIG. 43B) SDS-PAGE of representative fractions of the purification and FIG. 43C) the western blot of these fractions. MW, molecular weight marker (PageRuler™ Prestained Protein Ladder; and asterisk (*), commercial native human coagulation factor IX (used as control).

FIGS. 44A-44C. Purification of GFP-tagged glucocorticoid receptor (GFP-GR) in complex with molecular chaperones from transfected human HEK293T cells using Nb CA15816 as an immobilized trapper and Nb CA12670 as a stripper.

For the purification of GFP-GR by NANEX, CA15816 (medium-affinity trapper) was immobilized as a trapper on a HiTrap NHS-activated Sepharose HP column (1 mL). 10 mL of lysate was injected on this NANEX column. The loaded CA15816-column was washed twice with 10 CV of buffer (100 mM Hepes pH 7.5, 150 mM NaCl) followed by the injection of 1 mg of the stripper CA12670. FIG. 44A) The elution of GFP-GR by this stripper was monitored by following the absorbance at 280 nm and 488 nm. The high peak on the left eluted upon injection of the stripper (CA12670). The right peak eluted upon regeneration with 200 mM glycine at pH 2.3. FIG. 44B) SDS-PAGE of the different purification fractions, molecular weight marker (PageRuler™ Prestained Protein Ladder). FIG. 44C) Left, Three bands of high molecular weight (120 KDa, 90 KDa and 70 KDa) are visible in coomassie staining. These bands were analysed by mass spectrometry and confirmed to be GFP-GR (120 KDa), HSP90 (90 KDa) and HSP70 (70 KDa). Right, Western blot using commercial anti-human glucocorticoid receptor antibody (anti-GR G-5, Santa Cruz) as primary antibody confirmed that the band at 120 KDa is GFP-GR.

FIGS. 45A-45C. Purification of GFP-tagged androgen receptor (GFP-ARb) in complex with molecular chaperones from transfected human HEK293T cells using Nb CA15816 as an immobilized trapper and Nb CA12670 as a stripper.

For the purification of GFP-ARb by NANEX, CA15816 (medium-affinity trapper) was immobilized as a trapper on a HiTrap NHS-activated Sepharose HP column (1 mL). 10 mL of lysate was injected on this NANEX column. The loaded CA15816-column was washed twice with 10 CV of buffer (100 mM Hepes pH 7.5, 150 mM NaCl) followed by the injection of 1 mg of the stripper CA12670. FIG. 45A). FIG. 45A) The elution of GFP-GR by this stripper was monitored by following the absorbance at 280 nm and 488 nm. The high peak on the left eluted upon injection of the stripper (CA12670). The right peak eluted upon regeneration with 200 mM glycine at pH 2.3. FIG. 45B) SDS-PAGE of the different purification fractions, molecular weight marker (PageRuler™ Prestained Protein Ladder). Three bands of high molecular weight (128 KDa, 90 KDa and 70 KDa) are visible in coomassie staining. These bands were analysed by mass spectrometry and confirmed to be GFP-ARb (128 KDa), HSP90 (90 KDa) and HSP70 (70 KDa). FIG. 45C)

Western blot using commercial anti-GFP antibody (GFP Monoclonal Antibody (C163), ThermoFisher) as primary antibody confirmed that the band at 128 KDa is GFP-ARb.

FIG. 46. Information on 12 GFP-tagged yeast proteins chosen for high-throughput Nanobody exchange chromatography using magnetic beads.

This list provides detailed information, including the name and abbreviation, the reference number in the yeast GFP-clone collection (Huh et al., 2003), the molecular weight of the GFP fusion proteins, an estimation of the molecules per cell and the anticipated amount of protein produced in a yeast culture.

FIGS. 47A-47G. NANEX purification of GFP-fusion proteins from Yeast cell lysates using Nb CA15816 as an immobilized trapper on magnetic tosyl-activated Dynabeads®, and Nb CA12760 as a stripper.

12 GFP-fusion proteins were expressed and purified from different Yeast (S. cerevisae) clones from the yeast GFP-clone collection (Huh et al., 2003). Cell pellets from 1 mL cultures were lysed for 1 hour in, dialyzable Yeast Protein Extraction Reagent (Y-PER™ Plus) and spun after a freeze-thaw-cycle. The lysates were processed using a KingFisher Flex (ThermoFisher) instrument suitable for handling magnetic beads in 96 well format. Tosyl-activated magnetic Dynabeads® were coupled with the trapper Nb CA15816 at a concentration of 40 μg trapper/mg of beads according to the manufacturer's instructions. 5 μL of a 100 mg/mL solution of beads (corresponding to 20 μg of coupled trapper CA15816) was used per clone. The purification steps involved 30 seconds pre-equilibration of the beads, 30 minutes incubation with the lysate, 3 washes of 1 minute and 15 minutes elution with 40 μL stripper Nb CA12760 at 0.5 mg/mL concentration (33.35 uM). Samples of the CA15816-beads were harvested before the elution step to track the trapping efficiency. Panel A to C represent the SDS-PAGE analysis: (FIG. 47A) Proteins bound to the CA15816-beads. (FIG. 47B) Selective elution of GFP-fusion proteins using stripper Nb CA12760. (FIG. 47C) The corresponding CA15816-beads after the elution. The presence of the different GFP-fusion proteins tested was confirmed by western blot analysis (FIGS. 47D, 47E, 47F, resp.) and their respective molecular weight, using mouse anti-GFP Monoclonal Antibody (C163, ThermoFisher) as primary, goat anti-mouse IgG-HRP conjugate as secondary antibody, and SuperSignal™ West Atto Ultimate Sensitivity Substrate for chemiluminescence detection. M=molecular weight marker (PageRuler™ Prestained Protein Ladder).

FIG. 48: Detailed drawing of the microfluids chap that was used in Example 28.

All measures are in millimeters (mm).

FIG. 49: Fluorescence of the ii-fluidics column and the fractions eluted thereof as described in Example 28.

Panels A to F: Fluorescence images from the column contained in the μ-fluidics chip for the purification of GFP by NANEX, monitored using an inverted fluorescence microscope (Olympus IX71 model IX71S1F-3). Bottom panel 1. 2. 3. 4. 5. 6.: a. b. c. d. e. f. fractions that eluted from the microfluidics device were visually inspected using a blue light transilluminator (ThermoFisher).

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation, ubiquitination, sumoylation, and acetylation, among others known in the art. Based on the amino acid sequence and the modifications, the atomic or molecular mass or weight of a polypeptide is expressed in (kilo)dalton (kDa). By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or Nanobody as identified and disclosed herein which has been removed from the molecules present in the sample or mixture, such as a production host, that are adjacent to said polypeptide. An isolated protein or peptide can be generated by amino acid chemical synthesis or can be generated by recombinant production or by purification from a complex sample.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target protein or specific target component or molecule, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. Affinity is the strength of binding of a single molecule to its ligand. It is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants. At equilibrium, the rate of [antibody] [antigen] complex formation is equal to the rate of dissociation into its components [antibody]+[antigen]. The measurement of the reaction rate constants can be used to define an equilibrium or affinity constant (1/K_{D}). In short, the smaller the K_{D} value the greater the affinity of the antibody for its target. The rate constants of both directions of the reaction are termed: the association reaction rate constant (Kₒₙ), which is the part of the reaction used to calculate the “on-rate” (Kₒₙ), a constant used to characterize how quickly the antibody binds to its target. Vice versa, the dissociation reaction rate constant (K_{off}), is the part of the reaction used to calculate the “off-rate” (K_{off}), a constant used to characterize how quickly an antibody dissociates from its target. In measurements as shown herein, the flatter the slope, the slower off-rate, or the stronger antibody binding. Vice versa, the steeper downside indicates a faster off-rate and weaker antibody binding. The ratio of the experimentally measured off- and on- rates (K_{off}/Kₒₙ) is used to calculate the K_{D} value. Several determination methods are known to the skilled person to measure on and off rates and to thereof calculate the K_{D} (see below and examples), which is therefore, taking into account standard errors, considered as a value that is independent of the assay used.

As used herein, the term “protein complex” or “complex” or “assembled protein(s)” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions. A protein complex can be a non-covalent interaction of only proteins, and is then referred to as a protein-protein complex; for instance, a non-covalent interaction of two proteins, of three proteins, of four proteins, etc. More specifically, a complex of the protein binding agent and the target protein, optionally with other proteins or compounds bound to it.

A “binding agent” relates to a molecule that is capable of binding to another molecule, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. Preferably, the binding agent is a protein binding agent in the method described herein. The term “binding pocket” or “binding site” refers to a region of a molecule or molecular complex, that, as a result of its shape and charge, favourably associates with another chemical entity, compound, proteins, peptide, antibody, ISVD, or Nb. The term “pocket” includes, but is not limited to cleft, channel or site. The term “part of a binding pocket/site/epitope”, or “overlapping epitope” as interchangeably used herein, refers to less than all of the amino acid residues that define the binding pocket, or binding site, or epitope. For example, the portion of residues may be key residues that play a role in ligand binding, or may be residues that are spatially related and define a three-dimensional compartment of the binding pocket. The residues may be contiguous or non-contiguous in primary sequence. For antibody-related molecules, the term “epitope” is also used to describe the binding site, as used interchangeably herein. A ‘adjacent’ or ‘minimally overlapping’ binding site, as used herein, refers to ‘no overlapping amino acids (but binding to a site close by)’, or maximum of about 30% overlap in the binding amino acid residues respectively. An “epitope”, refers to an antigenic determinant of a polypeptide, constituting a binding site or binding pocket on a target protein molecule, which is an accessible epitope or binding site on the extracellular side. An epitope could comprise 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography, multi-dimensional nuclear magnetic resonance, Cryo-EM Hydrogen

Deuterium-Exchange (HDX)-MS, as well as Cross-linking Mass-spectrometry (XL-MS), epitope binning, or used to a lower extent also Neutron scattering, X-ray Free electron-laser (XFEL) or Small-angle neutron scattering (SANS) and small-angle x-ray scattering (SAXS) technology. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, ubiquitylation or alike, or attachments of hydrophobic groups, among others, can influence the conformation of a protein. Furthermore, environmental factors or conditions, such as temperature, pH value, salt concentration, ionic strength, and osmolality of the surrounding solution, and interaction with other proteins and co-factors, among others, can affect protein conformation and binding properties. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody”, “antibody fragment” and “active antibody fragment” as used herein refer to a protein comprising an immunoglobulin (Ig) domain or an antigen binding domain capable of specifically binding the antigen, or target protein epitope. ‘Antibodies’ can further be intact immunoglobulins derived from natural sources or from recombinant sources and can be immunoreactive portions of intact immunoglobulins. Antibodies are typically tetramers of immunoglobulin molecules. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains (ISVDs), Nanobodies, domain antibodies, and single chain structures, such as a complete light chain or complete heavy chain. An additional requirement for “activity” of said fragments in the light of the present invention is that said fragments are capable of specifically binding the target epitope. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1- CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, such as monoclonal antibodies, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4, said amino acid sequence -containing protein domain being sufficient for antigen or epitope binding, thus only requiring 3 CDR loop regions for interaction with its target epitope. The “active fragment” of ISVDs as described herein is defined as the portion of an ISVD that is sufficient to specifically bind the epitope in an identical or similar manner as the ISVD where this fragment is derived from binds to. An “immunoglobulin domain” of this invention also refers to “immunoglobulin single variable domains” (abbreviated as “ISVD”), equivalent to the term “single variable domains” and “single domain antibody”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody and their preparations can be found e.g. in WO 08/101985 and WO 08/142164. Nanobodies form the smallest antigen binding fragment that completely retains the binding affinity and specificity of a full-length antibody. Nbs possess exceptionally long complementarity-determining region 3 (CDR3) loops and a convex paratope, which allow them to penetrate into hidden cavities of target antigens. Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Moreover, further suitable mutations, in particular substitutions, can be introduced to generate a polypeptide with reduced binding to pre-existing antibodies present in human or animal cells (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined herein) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation, or for attachment of labels, such as biotinylation or fluorophores.

As used herein, the terms “determining,” “measuring,” “assessing,”, “identifying”, “screening”, and “assaying” are used interchangeably and include both quantitative and qualitative determinations.

### Detailed Description

The present invention relates to the purification of proteins by affinity chromatography. In particular, a pair of target-specific protein binding agents specifically binding an epitope on a target in a competitive manner, is used in a complementary kinetic context. Such a pair of binders which is competing in its target binding though with binding sites at non-overlapping or different epitopes, has been seen in affinity displacement to act via transient sandwich complexes and within defined dose- and kinetic relations. However, in cases where a pair of a first protein binding agent (trapper) with the same or overlapping epitope as the second binding agent is used, it will depend on the binding nature whether a cross-block or displacement can occur. Using ISVDs, or more specifically Nbs, as displacers, we found that even if a pair targeting the same epitope on a target protein was combined, under certain kinetic requirements, displacement is efficiently established, more specifically when the dissociation rate constant is higher for the first than the second protein binding agent, and wherein said second, known as the displacer or stripper, comprises an ISVD or Nanobody-specific binding nature. So for ISVD-binding agents used as displacer, the k_{off} seems to drive the displacement efficiency. This purification technology has been shown to function most optimally when the immobilized (trapper) protein binding agent is used with a higher off-rate (or k_{off} value) and/or a lower affinity as compared to the second ISVD-comprising protein binding agent for competitive elution of the target, the latter thus with a lower off rate and/or higher affinity for the target protein. Indeed, it is known from the art that antibodies or antibody domains, including ISVDs and Nanobodies compete for binding their targets when they interact with a similar or overlapping epitope. Purely based on the competitive nature, one could use the same binding agent, such as a Nanobody for binding (or trapping) and eluting (or stripping) to purify the target, hoping that competition allows to obtain a satisfying yield of purified protein in the elution fraction. Thus, binding agents with a dissociation rate allowing such ‘equal’ competition (i.e. k_{off} not too low, or affinity not too high, see below) will result in a certain amount of protein to be eluted using the same binding agent or Nb as trapper and stripper. However, in this case, the purification will not provide for the most optimal result, since the equally competing trapper will retain part of the protein bound to the immobilized surface, and elution yields will be suboptimal. The skilled artisan searching for a binding agent that is capable of fully outcompeting the trapper binding to the target, which is desired in high-throughput application, would find that conventional antibody binders to the same epitope mostly block any displacement reaction (e.g. as demonstrated for monoclonals in Abdiche et al. 2017), and one would rather use protein binding agents such as antibodies binding to an adjacent or minimally overlapping epitope as compared to the trapper, as to avoid such a block of the epitope by competition. Using as a second protein binding agent an ISVD, binding the same, substantially the same, or large overlapping epitope as the first protein binding agent, and wherein said second ISVD-comprising protein binding agent has a lower dissociation rate constant (k_{off}), showed that the ISVD was capable to efficiently displace the first protein binding agent, thereby outcompeting for binding to the same epitope of the target protein, and allowing elution of the target protein at high yields. The present invention hence relates to ‘ISVD-based displacement’, or more particularly ‘Nanobody exchange’ or ‘Nanobody exchange chromatography’ or ‘NANEX’, as interchangeably used herein, resulting in highly pure eluted protein complexes of said second ISVD-comprising protein binding agent with the target (as shown in the Examples). Even when a binding pair is used for displacement on a target whereby the epitopes are not or minimally overlapping, a displacement reaction, the ISVD binding nature seems to function according to displacement kinetics driven by a difference in k_{off} between capture and elution agent.

When using NANEX purification, the elution complex thus contains the stripper or displacer, which has the advantage that this allows to apply ISVD-comprising second protein binding agents (called strippers, or Nanostripper in the case of Nanobodies) that are additionally functionalized, i.e. they provide for a specific function to the eluted protein complex. Such a functionalization may relate to visualization of the protein complex (via fluorescence or labelling of the agent) or relates to functioning as a chaperone or adapter protein (including for instance but not limited to a MegaBody), among other examples, to elute the target in a functionalized complex. Moreover, following the elution step and a regeneration procedure, the affinity matrix, which may be any type of surface, such as beads, a column, or a resin, is ready for the next affinity purification cycle and can be used in high-throughput platforms, such as a screening platform, a chip, or a microfluidics setup or device.

By using this next-generation affinity purification technology, called NANEX or Nanobody exchange chromatography, a leap forward can be foreseen in analytical purification, as well as in high-throughput platform or screening applications such as screening assays, and structure-based drug design and discovery, as well as structure-based screening of novel compounds. In fact, protein binding agents with conformation-selective recognition of antigens or targets, to stabilize the target in a functional conformation, such as an active conformation, more specifically an agonist, partial agonist or biased agonist conformation can be selected for. With the rapid advancement of such technologies in biotechnology, it is foreseeable that the invention will impact the efficiency and potential of novel therapeutic drug screening as well as increase throughput and the potential of proteomics, MS-based, and other analytics.

A first aspect relates to a purification process for a target protein present in a sample, the process comprising the steps of:
a) Mixing a first protein binding agent that specifically binds an epitope on the target protein, with a sample containing said target protein,
b) Adding a second protein binding agent which competes for the target protein binding with the binding of the first binding agent, and
c) Elution of the protein complex comprising the target protein and the second protein binding agent,
   upon exchange or displacement of the target protein from the first to the second protein binding agent, said eluted protein complex comprising said target protein and said second protein binding agent, and wherein said second protein binding agent comprises and ISVD or a functional variant thereof, which is specifically bound to its epitope of the target protein, and wherein the dissociation rate (or ‘rate constant of dissociation’ or ‘k_{off}’, as used interchangeably herein) is slower or equal (or the k_{off} value lower or equal) for the second protein binding agent as compared to the k_{off} of the first protein binding agent. In further applications, said method may also comprise a washing step prior to addition of the second protein binding agent.

The term ‘functional variant’ of an ISVD is defined herein as any polypeptide that contains the binding region or paratope for binding the target protein that is identical to the binding region or paratope of the ISVD, so that the variant may differ in its sequence or composition, but retains its functionality in binding to the target protein with the same binding region as the ISVD. In particular, this paratope or binding region of an ISVD most often comprises at least the CDR3 region, preferably 3CDRs, and occasionally also part of the FR regions.

The feature as to ‘compete for the target binding’ may be interpreted as competing for the same epitope, or may also mean competing in a different manner, such a kinetically or allosterically. So in one embodiment, the stripper may compete for binding by targeting minimally overlapping or adjacent epitopes, or alternatively, the stripper can even disrupt the interaction between the trapper and the target by binding to an allosteric site on the target, by inducing a conformation change of the target. Competing binding agents may be established using several methods as known in the art, for example, but not limited to, a competition ELISA, alphalisa, Octet measurements or bio-layer interferometry (BLI), SPR Biacore, Microscale thermophoresis (MST), amongst others.

More specifically, to obtain an efficient displacement in the purification method, a difference in requirements may be considered for binding pairs which compete for the target through binding to the same or largely overlapping epitope, versus binding pairs which compete for the target through binding to minimally or non-overlapping epitopes, for instance by binding to adjacent epitopes, or by binding to an allosteric site which induces conformational changes forcing the displacement. The displacement reaction using the first type of pairs, wherein the ISVD-containing stripper binds a similar epitope is driven by the difference in k_{off}, and hence requires a displacer with a lower k_{off} value as compared to the capturing agent, which often also results in a stripper with a higher affinity as the trapper. So herein, the displacement is not driven by the association rate constant.

The displacement reaction using the second type of pairs, wherein the ISVD-containing stripper binds a different or minimally overlapping epitope is also driven by a difference in k_{off}, or affinity, but allows for a more gentle difference, and shown for instance by just a 2-fold difference to allow displacement. In a specific embodiment, said binding agents are not monoclonal or conventional antibodies, as this will require different displacement kinetics.

In said purification method, binders with the ‘same’ epitope are defined herein as that the amino acid residues of the target protein interacting with said binding agents are identical, wherein ‘interacting’ or ‘in contact’ with the binding agent is described as closer than 3 Å from said residue (or atom) upon binding of the binding agent with said target protein at said epitope. The term ‘substantially the same’ or ‘largely overlapping’ epitope as described herein refers to the number of identical amino acid residues of both epitopes being at least 50%, at least 60%, at least 70%, at least 80%, at least 90%, at least 95%, or at least 99% of the total or highest number of amino acid residues of the epitopes. Most preferably, ‘substantially the same’ epitopes referring to the number of identical amino acid residues of both epitopes being at least 85-99% over the highest number of amino acid residues of the epitopes. Most preferably, ‘largely overlapping’ epitopes referring to the number of identical amino acid residues of both epitopes being at least 50-84% over the highest number of amino acid residues of the epitopes. Further, said purification method delivers the most optimal result when the first binding agent or trapper has a higher dissociation rate or lower or equal affinity as compared to the second binding agent, and vice versa, when the second binding agent has a lower dissociation rate and/or the same or higher affinity for the epitope as compared to the first binding agent. From current state of the art knowledge, the rate constant of dissociation (or off-rate or k_{off}) and the rate constant of association (or on-rate or kₒₙ) are interrelated as K_{D}=k_{off}/kₒₙ, wherein K_{D} is defined as the dissociation constant, which is inversely correlated with the affinity of a binding agent for its target, as described also in detail in the definitions above. So, if the dissociation constant K_{D} value is low(er), the affinity is high(er) (if kₒₙ is the same). Alternatively, if kₒₙ is higher, the K_{D} is lower and the affinity is higher (if k_{off} is the same).

So, for the purification method of the present invention, the protein binding agents described herein are relatively different in k_{off} and/or affinity (or K_{D}) for the same, substantially the same or largely overlapping epitope. The method as described herein refers more specifically to the k_{off} of the second binding agent being lower than the k_{off} of the first binding agent for the same, substantially same, or largely overlapping epitope of the target protein, with ‘lower’ referring herein to a value that is at least 2-fold lower, 5-fold lower, or 10-fold lower, or at least 30-fold lower, or at least 100-fold lower, or at least 200-fold, at least 300-fold, at least 400-fold, or at least 500-fold lower. More preferably, said k_{off} value of the second binding agent is in the range of at least 2-fold lower to at least 10-fold lower, or at least 5-fold lower to at least 20-fold lower, or at least 10-fold lower to at least 30-fold lower, or at least 100-fold lower, as compared to the k_{off} value of the first protein binding agent.

Similarly, the affinity of the second binding agent may be equal or higher than the affinity of the first binding agent for the epitope of the target protein, wherein ‘higher affinity’ refers to a ‘K_{D} value’ of the second protein binding agent being a K_{D} value that is at least 2-fold lower, or at least 5-fold lower, 10-fold lower, 20-fold lower or 100-fold lower, or in the range of at least 2- to at least 2000-fold lower, as compared to the K_{D} value of the first protein binding agent.

In a preferred embodiment, the purification method as described herein discloses a first binding agent with a K_{D} value for the epitope of the target protein of 1 mM to about 1 nanomolar and discloses a second protein binding agent with a K_{D} value, optionally with substantially the same or largely overlapping epitope for said target, of 1 nanomolar or lower, optionally down to 1 picomolar. More preferably, said first binding agent has a K_{D} in the nano- to millimolar range (i.e. 10E−9 to 10E−3) and the second binding agent has a K_{D} value in the femto-to micromolar range (i.e. 10E−12 to 10E−6), most preferable with a relative difference between the first and second binding agent of at least 2-fold. In one embodiment said K_{D} value for the first protein binding agent is at least 2-fold higher than the K_{D} of the displacer, a difference which is driven by the difference in k_{off} value, especially when the displacer binds to the same or largely overlapping epitope.

The method of the present invention comprises a second protein binding agent, which is in solution, and soluble in elution conditions. Said elution conditions preferably relate to physiological conditions, as known to the skilled person. The term ‘soluble’ as used herein refers to the fact that the protein binding agent is in a functional form, meaning that it is capable of specifically binding its target within the expected range of its affinity for the epitope. Said method of purification comprises said first protein binding agent, which may be present as a free, labelled, or covalently bound protein binding agent in a solute for mixing with the sample of interest. Said first binding agent may for instance be coupled to beads, which may be agarose or magnetic beads, or may be present on a surface or matrix, more specifically on packed as an affinity column, which may be suited for preparative as well as analytical purification scales, more particularly, which may be a microcolumn in the order of below 1 mL column volume, or even in submicromolar volumes, or even provided on a chip using microfluidics technology. Said first protein binding agent is preferably immobilized for the method of the present invention, wherein it may be immobilized on a surface via covalent or other means of coupling. Most preferably, said first protein binding agent is immobilized on a solid support or resin. A ‘resin’ or ‘affinity resin’ as used interchangeably herein, is an activated affinity chromatography support for the immobilization of biomolecules such as ISVDs or other protein binding agents. In a specific embodiment, said first protein binding agent comprises an ISVD and is coupled to a resin using known coupling methods from the art (see examples).

The method of purification as described herein comprises the steps of mixing the first protein binding agent, optionally immobilized, with a sample in step a), wherein said sample comprises the target protein specifically binding the first protein binding agent. In specific embodiments said sample may relate to a biological sample, a biopsy sample, a cellular or tissue-containing sample, a cellular lysate, a mixture of cells, or a complex mixture, solvent or lysate comprising non-specified components. Other embodiments relate to synthetic or non-natural compound containing samples, composed samples, or other in vitro samples.

Depending on the nature of the sample, the optional washing of the column after step a) of the method of the present invention may require neutral, or very mild, or rather harsh conditions, or may require repetitions to remove any unbound abundant components. Depending on the nature of the sample, and the amount of target protein present in the sample, as well as on the first protein binding agent following step a) or b) of the method of the present invention, the elution in step c) using the second protein binding agent may be repeated, or may require larger volumes of elution as to allow complete elution of the target protein. The method as presented herein has the advantage that upon said elution, using physiological conditions, and optionally including a mild regeneration step, the affinity column (i.e. the first protein binding agent immobilized on a resin) is reusable for further purifications from additional samples.

When the eluate of step c) of the method of the present invention requires a higher purity than what is obtained after the purification step of the method described herein, an additional purification step is preferable, wherein the same steps of the purification method as described herein are repeated using a a third and fourth protein binding agent, which both bind to the same epitope of the target protein, said epitope being non-overlapping, adjacent or different from the epitope bound by the first and second protein binding agent. Said method for tandem purification of a target protein comprising the steps of
a) mixing a first protein binding agent specifically binding an epitope of a target protein with a sample containing said target protein,
b) adding a second protein binding agent, competing for the target protein with the first binding agent, to displace the first binding agent from the target protein by specifically binding the target protein,
c) collecting the elution complex comprising the second protein binding agent bound to the target protein, and
d) repeating steps a) to c), using a 3^{rd} and 4^{th} protein binding agent instead of the 1^{st} and 2^{nd} protein binding agents, respectively, wherein said 3^{rd} and 4^{th} binding agent specifically bind a different epitope of said target protein as compared to the epitope for the 1^{st} and 2^{nd} binding agent, and
   wherein the second (and/or fourth) protein binding agent comprises an immunoglobulin single variable domain (ISVD) or an active fragment thereof specifically binding the epitope, and wherein the rate constant of dissociation (k_{off} value) of the second and fourth protein binding agent is lower or equal as compared to the k_{off} value of the first and third protein binding agent, respectively.

In fact, such a tandem-NANEX affinity purification method is suitable for generically purify protein complexes comprising more than one target protein as well, as is also aimed for the classical tandem affinity purification (TAP) using a TAP tag. Indeed, the epitope recognized by the 3rd and 4th protein binding agent may also be present on a target protein which is different from the target protein binding the 1st and 2nd binding agent, but which will allow to capture and purify a complex formed between said first and second target protein, even for purifying such a protein-protein interaction from highly complex matrices, where a one-step purification step is not sufficient. The advantage of this tandem affinity purification method over the known TAP methods in the art is that the purification does not require enzymatic cleavage (of a tag), and no remaining protease is present in the eluate. Further advantages of this method include that there is no need for a concentration step or dialysis to certain buffer conditions, and that excess of the 2nd protein binding agent or stripper Nb can be removed in the second step of the tandem approach. Alternatively, a tandem-NANEX may be envisaged wherein the first and third binding agent are both coupled on the same column or resin, and the tandem is exerted as a type of multiplex reaction, using the second and fourth as strippers simultaneously or subsequently, optionally allowing a washing step in-between.

Finally, the purification method as described herein preferably applies a trapper/stripper pair wherein the protein binding agents both comprise ISVDs, or more specifically VHHs, or even more specifically Nanobodies, since this type of protein binding agents has the advantageous properties to be highly specific, well expressed in E. coli/Pichia, high (thermal) stability, and can be selected for salt/pH tolerance of the binding affinity, and all bind to their targets via a single variable domain as described herein, allowing to apply similar displacement kinetics for this class of ISVD-containing strippers.

Another embodiment relates to the method of purifying a target protein as disclosed herein, wherein the second (or fourth) protein binding agent comprises a label or detectable label. The term “detectable label” or “labelling”, refers to detectable labels or tags allowing the detection, visualization, and/or isolation, further purification and/or immobilization of the isolated or purified (poly-)peptides or complex described herein, and is meant to include any labels/tags known in the art for these purposes. Particularly preferred are fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase; and (other) enzymatic labels (e.g., peroxidase, alkaline phosphatase, beta-galactosidase, urease or glucose oxidase). Also included are affinity tags, such as chitin binding protein (CBP), maltose binding protein (MBP), glutathione-S-transferase (GST), poly(His) (e.g., 6x His or His6), Strep-tag®, Strep-tag II® and Twin-Strep-tag®; solubilization tags, such as thioredoxin (TRX), poly(NANP) and SUMO; chromatography tags, such as a FLAG-tag; epitope tags, such as V5-tag, myc-tag and HA-tag. Also included are combinations of any of the foregoing labels or tags. The second (or fourth) protein binding agent may, for example, be fused or conjugated to a half-life extension module, or may function as a half-life extension module itself. Such modules are known to a person skilled in the art and include, for example, albumin, an albumin-binding domain, an Fc region/domain of an immunoglobulins, an immunoglobulin-binding domain, an FcRn-binding motif, and a polymer. Particularly preferred polymers include polyethylene glycol (PEG), hydroxyethyl starch (HES), hyaluronic acid, polysialic acid and PEG-mimetic peptide sequences. Modifications preventing aggregation of the isolated (poly-)peptides are also known to the skilled person and include, for example, the substitution of one or more hydrophobic amino acids, preferably surface-exposed hydrophobic amino acids, with one or more hydrophilic amino acids.

In a further embodiment, protein binding agents specifically binding an epitope on different types of target proteins are described, wherein said epitope may for instance constitute or comprise a tag as present on a fusion protein. Examples of tags are herein included but not limited to affinity tags such as commonly used Polyhistidine (His), gluthatione transferase (GST), maltose binding protein (MBP), calmodulin binding peptide (CBP) , intein-chitin binding domain (intein-CBD), Streptavidin/Biotin-based tags, His-Patch ThioFusion (thioredoxin based), EPEA (CaptureSelect C-tag; US9518084B2), ubiquitin, or Small ubiquitin-like modifier (SUMO), yeast SUMO or SMT3, or HaloTag. Furthermore, tags may constitute epitope tags, such as HA, FLAG, or cMyc, or even reporter tags, such as HRP, or Alkaline phosphatase, though the latter being less preferred for affinity purification. A number of non-limiting examples is provided for example in Kimple et al. (2015 Table 9.9.1).

Alternatively, the protein binding agents of the method for purification described herein specifically bind an epitope of a native, a naturally occurring, and/or an endogenous protein, not requiring a fusion to a tag. Another alternative is that the epitope is present on a recombinantly produced exogenous protein, not requiring a tag. For such protein binding agent pairs, in order to compete for the same target, one may screen and select to provide for a pair of competing binding agents, or one may design towards a higher affinity and lower affinity pair of protein binding agents. Indeed, using a 3D-structure of the stripper or 2nd protein binding agent bound to the target allows to design mutations in the binding site of the protein binding agent that will result in a reduced affinity or higher k_{off}, and thereby provide for compatible trapper or 1st protein binding agent. Besides, more straightforward methods, not requiring structural information also allow to determine pairs based on a single binding agent, once the sequence is known. As shown in the examples, in a non-limiting way, for the mCherry target protein, based on a screening for different binders, their competing nature was analysed by epitope mapping using BLI, or alternatively, based on the sequence of a nanomolar binder, or stripper, using an alanine mutation scan in the CDR3 region, known to be most critical in defining the binding kinetics, new pairs with lower dissociation rate constants could be identified, to function as trappers in the NANEX method. In this way, pairs of protein binding agents binding the same epitope with a different k_{off} or affinity will be obtained simply by introducing single or multiple mutations.

In the specific embodiment relating to a method wherein not only the second but also the first (and optionally the 3rd and 4th) protein binding agent comprise an ISVDs, the ‘monovalent’ format may be used as a trapper (first or third binding agent), and a ‘multivalent’ format in combination as a stripper (2nd or 4th binding agent), since multivalent formats have a higher avidity as compared to the monovalent forms, with higher k_{off}, resulting in optimal elution yields and target protein purity as well (see Example 12). The term ‘monovalent format’ herein refers to an ISVD, as used herein, that can only recognize one antigenic determinant, while the term ‘multivalent’ format refers to an ISVD as used herein that can recognize more than one antigenic determinant, such as—but not limited to—bivalent, trivalent or tetravalent formats. Moreover, instead of a multivalent stripper, also a multiparatopic or multispecific stripper may be envisaged, wherein said stripper may comprise an identical building block binding to the same antigenic determinant, and at least one or more building blocks binding that may be different and may bind the same or another epitope on the target protein, or alternatively, an epitope on another target protein in complex with the first target protein.

In a further embodiment, the method for purification of the target protein applies a MegaBody as a first and/or second protein binding agent. The term MegaBody as used herein refers to the novel fusion proteins disclosed in Steyaert et al. (WO2019/086548A1), also called antigen-binding chimeric proteins herein, referring to the fusion protein comprising an antigen-binding domain, which is connected to a scaffold protein, wherein said scaffold protein is coupled to said antigen-binding domain at one or more amino acid sites accessible or exposed at the surface of said domain, resulting in an interruption of the topology of said antigen-binding domain. Said antigen-binding chimeric protein is further characterized in that it retains its antigen-binding functionality as compared to the antigen-binding domain not fused to said scaffold protein. The MegaBody as described herein relates to the particular MegaBody or antigen-binding chimeric protein for which the antigen-binding domain comprises an immunoglobulin single variable domain (ISVD) or a Nanobody, which is fused or connected to a scaffold protein, at an accessible surface of said ISVD domain (β turn or loop, excluding the CDRs), resulting in an interruption of the topology of said antigen-binding domain, and retaining its antigen-binding functionality, i.e. the specific epitope recognition. In a specific embodiment, said second protein binding agent relates to the MegaBody or antigen-binding chimeric protein comprising an ISVD connected to the scaffold protein via an insertion of the scaffold protein in the first beta-turn connecting the beta-strand A and B of the ISVD (as defined according to IMGT nomenclature, and as defined in WO2019/086548A1). In an even more specific embodiment, the scaffold protein used herein is the HopQ or Ygjk scaffold protein, wherein the fusion of the scaffold interrupts the topology of the ISVD, but not its overall 3D-structure, neither its epitope-binding specificity. The ‘HopQ’ or ‘HopQ-derived’ scaffold as used herein relates to a protein scaffold of the Adhesin domain of the type 1 HopQ of Helicobacter pylori strain G27 (Protein Database: PDB 5LP2), or a circularly permutated protein thereof, also called cHopQ or c7HopQ (see also WO2019/086548A1). The ‘Ygjk’ or ‘Ygjk-derived’ scaffold as used herein relates to a protein scaffold of the Escherichia coli K12 YgjK (PDB 3W7S), or a circularly permutated gene encoding said protein thereof, also called cYgjk (see also WO2019/086548A1).

The embodiment relating to said method wherein the second protein binding agent is a MegaBody, specifically binding the epitope of the target protein via its ISVD antigen-binding domain, results in the elution of the target protein bound to said MegaBody. As disclosed in Steyaert et al. (WO2019/086548A1), as well as in Laverty et al. (2019), Masiulis et al. (2019) and Uchanski et al (2019), these exemplified Megabodies act as a novel type of Nanobody-based chaperones for improved structural resolution in cryo-EM analysis. So, the method of purification applying a MegaBody as described herein as second or fourth protein binding agent, i.e. as a stripper, is advantageous for straightforward preparation and purification of complex samples for cryo-EM or other structural analyses. The k_{off} of the ISVD comprised in said MegaBody is lower than the k_{off} of the first protein binding agent, which constitutes another protein binding agent binding the same, substantially the same or largely overlapping epitope.

In fact, a specific embodiment relates to a method for purification of a target protein comprising the steps of: a) mixing a first protein binding agent, comprising an ISVD, specifically binding an epitope of a target protein with a sample containing said target protein, followed by optionally, washing the mixture of step a) to remove non-bound sample components, and b) adding a second protein binding agent recognizing the same or largely overlapping epitope of said target protein as the first binding agent, to displace the first binding agent from the target protein by specifically binding the target protein, and c) collecting the eluate comprising the bound target protein to the second protein binding agent, wherein said second protein binding agent comprises a MegaBody, as described herein, comprising an ISVD specifically binding the same, substantially the same or largely overlapping epitope as the first protein binding agent, and wherein the rate constant of dissociation (koff value) of said MegaBody is lower, and its affinity is equal or higher as compared to the first ISVD-comprising binding agent. In a specific embodiment said ISVD of the first binding agent is a mutant ISVD of the ISVD comprised in the MegaBody. Said method of purification ultimately provides for a single step purification from, for instance, complex cellular sample, or small sample mixtures for use in structural biology and physicochemical characterization or analytical studies.

Another embodiment relates to the method of purification of a target protein as described herein, wherein the epitope recognized by the first and second protein binding agent relates to a protein binding site or epitope present on a scaffold protein as comprised in a Mega Body. Said Mega Body may preferably be built using a scaffold protein derived from HopQ or Ygjk protein, hence said HopQ or Ygjk protein scaffolds containing the epitope specifically binding to the protein binding agents of the method. Said pair of protein binding agents specifically binding the scaffold protein epitopes present on a MegaBody as disclosed herein, or as disclosed in Steyaert et al. (WO2019/086548A1), or as may be described elsewhere has the further advantage that the purification method can be applied to capture or scavenge MegaBody-bound target protein from complex mixtures.

Furthermore, if said protein binding agents specifically binding said epitope of HopQ or Ygjk scaffold proteins would still bind HopQ and Ygjk when fused to other proteins, or via other fusion formats, besides MegaBody fusions, the pair of HopQ- and/or Ygjk-specific protein binding agents can be used in said method for further applications requiring purification of said HopQ- or Ygjk-based fusion proteins, in a similar manner as for the tags discussed herein.

Altogether the method as presented herein further provides for another aspect of the invention, which constitutes the pairs of binding agents itself, as to allow a full-blown toolbox for analytics of endogenous (often present in small amounts) and difficult to isolate proteins, as well as analytical tools for tagged proteins, to further miniaturize the method on for instance microfluidics chips to allow HTP analytical application in Mass spec and structural analysis. So a second aspect of the invention relates to a kit comprising the first and second protein binding agent of the method for purification of a target protein, wherein said second protein binding agent comprises an ISVD or a functional variant thereof, and wherein the rate constant of dissociation of the second protein binding agent is equal or lower as compared to the first binding agent and competed for the target protein. Said kit may further comprise buffers for solubilizing, washing or eluting, or a resin. Furthermore, instructions or protocol of performing the method of purification may be provided in said kit. The first and second protein binding agent of said kit relate to a protein binding agent, the second one being defined as the one with the lower dissociation rate or alternatively, the higher affinity. If more than 2 protein binding agents are present in said kit, these multiple protein binding agents may specifically bind the same, substantially the same or largely overlapping epitope, or different epitopes, and/or may differ in format (ISVD comprising and/or functional variants thereof) and antibodies, peptides, among others as a first/third binding agent. Alternatively, said kit comprising multiple protein binding agent for the method of purification of a target protein as described herein, may comprise protein binding agents binding to different epitopes or even different proteins (e.g. of a protein complex), to allow purification of a mixture of proteins or of a protein complex. Said multiple protein binding agents however should at least be present in pairs as described herein to allow for using them in the purification or tandem purification method as described herein. In another embodiments said kit comprising said binding agents may comprise the first binding agent or trapper in an immobilized format, present on a solid structure, such as beads, resin, or in a chip.

In a specific embodiment, said kit comprises a first and second protein binding agent for use in the method of purification of a GFP-tagged protein, wherein said protein binding agent is selected from the group of SEQ ID NO: 1-6, 18 or 19 (or comprising any of these sequences without the his-EPEA tag) or a sequence with a homologous amino acid sequence of at least 70%, or at least 80%, or at least 90%, or at least 95%, or at least 99% identity thereof, and wherein the first and second binding agent must not be identical in said selected sequence of the KD of said ISVD is below 0.1nM, or more specifically if SEQ ID NO: 1 is selected. Similarly, the kit may comprise other protein binding agents for use in the method as described herein, wherein the agents specifically recognized a commercially available tag for a protein, such as GST, EPEA, mCherry, Ubiquitin, SMT3 or others, as exemplified and described herein.

Another embodiment relates to the use of said kit for the method as described herein, for purification or analysis purposes, as well as the use in a screening assay for instance in which binders for a druggable conformation are screened for.

A further aspect of the invention relates to a protein complex comprising the second (or fourth) protein binding agent of the method of purification, bound to the target protein. Said protein complex is provided for in the elution step c) (or repeat of c) in d)) of the method as described herein, which provides for the sample containing the protein complex of interest for further analysis. Indeed, the (second or fourth) protein binding agent eluted in complex with the target protein may provide for a stabilizing chaperone required for high-resolution structural analysis, and/or may provide for a stabilizing effect for certain target protein conformations, and/or may provide for an increased protein mass required for atomic resolution cryo-EM microscopy imaging, or may provide for a visually detectable complex, for instance when a labelled protein binding agent was used, or may provide for alternative applications, such as mass spec analysis, and is not limited by the examples provided herein. The elution fraction of step c) (or repeat of c) in d)) of the method described herein does provides at least for the protein complex, but may also contain additional buffer components, residual impurities, and/or components added to the elution solution to provide for suitable analytical conditions for the protein complex. Furthermore, said complex comprising the target protein of interest and the displacer, may also contain further proteins bound to said target protein, as part of a protein-protein complex that is isolated from the sample through NANEX purification.

A specific embodiment relates to the protein complex wherein the epitope of the target protein, which is recognized by and bound to the 2^{nd} or 4^{th} protein binding agent is a protein comprising a tag or consisting of said tag, as selected from the list of GFP, mCherry, GST, EPEA, SMT3, among others as listed herein. Hence said protein complex comprises the second (or fourth) protein binding agent, and the target protein comprising or constituting said tag as selected from said group. Furthermore, said protein complex may be a crystalline complex or a crystal.

A further aspect relates to the use of said protein complex as described herein for structural analysis, structure-based drug design, drug discovery, mass spectrometry, but also as a diagnostic tool, or for in-vivo imaging. Indeed, quantitative mass spectrometry analysis of low abundant proteins present in complex mixtures is often difficult and unreliable due to the low signal to noise ratio. So, the method of purification as described herein may be advantageous to provide for highly pure analytical samples of such target proteins for MS profiling, or for identification of interacting protein partners.

Furthermore, the protein complex as described herein may provide for a 3-dimensional structural representation at atomic resolution of said complex, in a high resolution, preferably with a resolution between 0.1 and 3 Å, obtained by cryo-EM structural analysis. In an alternative embodiment, the crystalline protein complex as described herein may specifically relate to a crystal of a GFP-specific Nanobody and GFP target protein, said GFP-specific Nanobody as depicted in SEQ ID NO: 1 or a sequence with at least 90%, at least 95%, or at least 99% identity thereof, and said GFP protein as depicted in SEQ ID NO: 16, or a sequence with at least 90% , at least 95%, or at least 99% identity thereof, wherein said crystal is characterized in that the crystal lattice constants are: a=74.497±5% b=103.450 ±5% c=209.774±5% Å α=90.00° β=90.00° [symbol] =90.00° (Space group P212121).

From said crystal structure, the binding site or epitope on the GFP protein where said Nb as depicted in SEQ ID NO: 1 is interacting with, consist of a subset of atomic coordinates, providing for the binding site consisting of amino acids residues number PRO89, GLU90, GLU111, LYS113, PHE114, GLU115, GLY116 of SEQ ID NO: 16.

Said binding site or epitope as determined by the 3D-structural representation of crystal may further allow to design and generate mutant protein binding agents, such as mutant ISVDs, to reduce their affinity, or increase their k_{off} as compared to the protein binding agent or ISVD present in the protein complex. For designing such a mutant variant, which will differ in at least one amino acid from the protein binding agent present in the complex, a skilled person will rely on (computer-assisted) methods available in the art as to obtain a higher k_{off} and/or lower affinity for the epitope on the target protein, as defined herein. So using the 3D structure of the protein complex as described herein, the skilled person may create mutation(s) in the binding agent its binding domain of the 3D structure using a computer-assisted method, further allowing him to display a superimposing model of said mutated binding domain on the three-dimensional model, and finally allowing him to assess whether said mutated binding domain results in a higher k_{off} and/or lower affinity to the target protein. As presented herein in Examples 1 and 2, the GFP binding agents were designed in a similar manner.

Finally, the application of the method and means of the present invention for use in structure-based drug design, drug discovery and structure-based drug screening is also encompassed herein. The iterative process of structure-based drug design often proceeds through multiple cycles before an optimized lead goes into phase I clinical trials. The first cycle includes the cloning, purification and structure determination of the target protein or nucleic acid by one of three principal methods: X-ray crystallography, NMR, or homology modeling. Using computer algorithms, compounds or fragments of compounds from a database are positioned into a selected region of the structure. One could use the purification method and the ISVD-based protein binding agent of the invention to purify, fix and/or stabilize certain structural conformations of a target. The selected compounds are scored and ranked based on their steric and electrostatic interactions with this target site, and the best compounds are tested with biochemical assays. In the second cycle, structure determination of the target in complex with a promising lead from the first cycle, one with at least micromolar inhibition in vitro, reveals sites on the compound that can be optimized to increase potency. Also at this point, the purified protein complex of the invention may come into play, as it facilitates the structural analysis of said target in a certain conformational state. Additional cycles include synthesis of the optimized lead, structure determination of the new target:lead complex, and further optimization of the lead compound. After several cycles of the drug design process, the optimized compounds usually show marked improvement in binding and, often, specificity for the target. A library screening leads to hits, to be further developed into leads, for which structural information as well as medicinal chemistry for Structure-Activity-Relationship analysis is essential. Applying protein binding agents as described herein that comprise an ISVD such as a Nanobody or MegaBody offer the additional advantage for said drug discovery method that only average images of correctly folded target proteins will be encompassed because the selection for displayed targets using Nanobodies or MegaBodies reveals mostly binders to conformational epitopes. According to a particularly preferred embodiment, the above described method of identifying conformation-selective compounds is performed by a ligand binding assay or competition assay, even more preferably a radioligand binding or competition assay. Most preferably, the above described method of identifying conformation-selective compounds is performed in a comparative assay, more specifically, a comparative ligand competition assay, even more specifically a comparative radioligand competition assay. The compounds to be tested can be any small chemical compound, or a macromolecule, such as a protein, a sugar, nucleic acid or lipid. Typically, test compounds will be small chemical compounds, peptides, antibodies or fragments thereof. It will be appreciated that in some instances the test compound may be a library of test compounds. In particular, high-throughput screening assays for therapeutic compounds such as agonists, antagonists or inverse agonists and/or modulators form part of the invention. Methodologies for preparing and screening such libraries are known to those of skill in the art. The test compound may optionally be covalently or non-covalently linked to a detectable label. Suitable detectable labels and techniques for attaching, using and detecting them will be clear to the skilled person, and include, but are not limited to, any composition detectable by spectroscopic, photochemical, biochemical, immunochemical, electrical, optical or chemical means.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The test compound as used in any of the above screening methods is selected from the group comprising a polypeptide, a peptide, a small molecule, a natural product, a peptidomimetic, a nucleic acid, a lipid, lipopeptide, a carbohydrate, an antibody or any fragment derived thereof, such as Fab, Fab′ and F(ab′)2, Fd, single-chain Fvs (scFv), single-chain antibodies, disulfide-linked Fvs (dsFv) and fragments comprising either a VL or VH domain, a heavy chain antibody (hcAb), a single domain antibody (sdAb), a minibody, the variable domain derived from camelid heavy chain antibodies (VHH or Nanobody), the variable domain of the new antigen receptors derived from shark antibodies (VNAR), a protein scaffold including an alphabody, protein A, protein G, designed ankyrin-repeat domains (DARPins), fibronectin type III repeats, anticalins, knottins, engineered CH2 domains (nanoantibodies), as defined hereinbefore. In one preferred embodiment, high throughput screening methods involve providing a combinatorial chemical or peptide library containing a large number of potential therapeutic ligands. Such “combinatorial libraries” or “compound libraries” are then screened in one or more assays, as described herein, to identify those library members (particular chemical species or subclasses) that display a desired characteristic activity. A “compound library” is a collection of stored chemicals usually used ultimately in high-throughput screening. A “combinatorial library” is a collection of diverse chemical compounds generated by either chemical synthesis or biological synthesis, by combining a number of chemical “building blocks”. Preparation and screening of combinatorial libraries are well known to those of skill in the art. The compounds thus identified can serve as conventional “lead compounds” or can themselves be used as potential or actual therapeutics.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### General Introduction

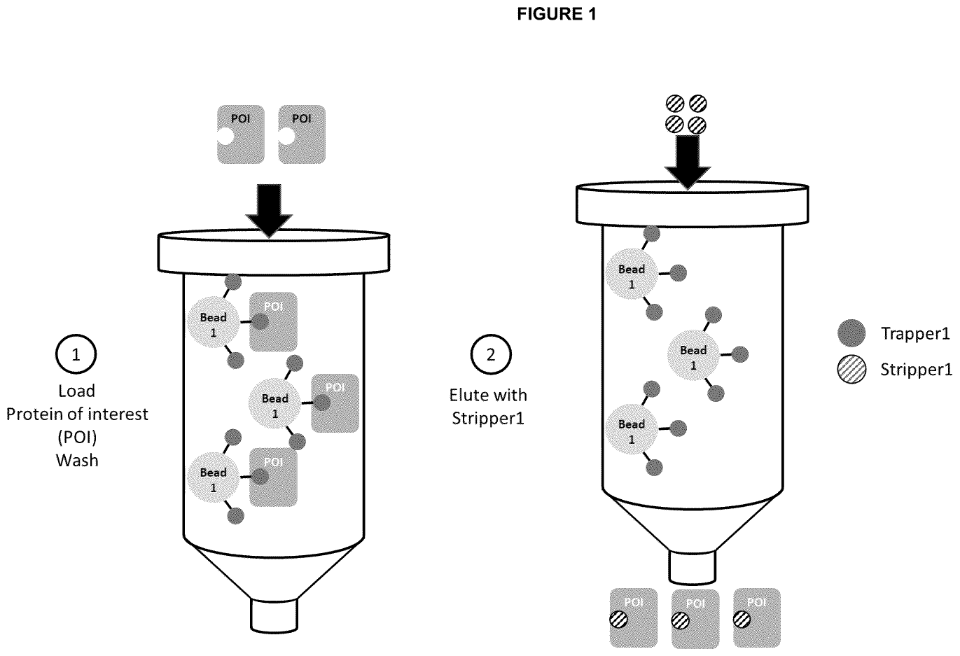

Nanobody exchange chromatography (NANEX) is described herein for the first time and is based on the principle of affinity-displacement chromatography, specifically developed herein for binding agents competing for the target protein binding in exchange chromatography, and using ISVD-containing displacer agents. FIG. 1 pictures the principle of the competitive affinity exchange by the use of Nanobodies as binding agents for a Protein of interest (or target protein, as interchangeably used herein). The protein of interest is in first instance captured by a trapper antigen-binding protein, or in particular a Nanobody, which may be immobilized, optionally followed by a washing step, and subsequently specifically eluting the protein of interest by addition of an elution buffer which contains the second binding agent, which acts as a displacer or stripper, and specifically binds the antigen via its immunoglobulin single variable domain recognizing the target protein epitope. The eluted complex is obtained through competing kinetically with the first binding agent or trapper, and as shown herein, allows to apply strippers which compete by binding the same or a largely overlapping epitope as the first bound Nb, or compete by binding the target at a minimally overlapping or even different epitope through an allosteric or kinetic difference. The main kinetic requirement for using an ISVD or functional variant thereof as a displacer relates to the dissociation rate constant (k_{off}), which is preferably lower than the k_{off} of the trapper. This often results in a higher affinity for the stripper as compared to the trapper. The dose-dependency also allows displacement even if the same binder is used for trapping and stripping, so an equal k_{off} value for the reaction, though this is only providing for a relatively efficient reaction when the dissociation rate constant (k_{off}) is at least 0.0001 s⁻¹, resulting in a half-time of less than 2 hours, which allows good trapping and stripping. More specifically, the k_{off} may also be lower than 0.0001 s⁻¹, which will result in displacement, though with a longer dissociation half-time, and therefore less favorable. In a specific embodiment the k_{off} may be at least 0.00005, or 0.00001 s⁻¹. In another embodiment said k_{off} may be in the range of 0.0001 to about 0.05 s⁻¹.

In general, due to the addition of this second competing Nb to the mixture/column where the first binding agent binds the target, the binding of the target to the first Nb will be disturbed and the target protein will release from the first protein binding agent or Nb, through competing binding for the second protein binding agent or Nb, resulting in elution of the protein of interest. Although one would expect, as shown for other antibodies such as monoclonal antibodies, that in cases where epitopes are largely overlapping competition may lead to a block of the first binding agent for the epitope of the second binding agent, the kinetic mechanism for the ISVD-driven displacement to the target was shown to be different. Moreover, we showed that an optimal kinetic ratio between said binders for trapping and stripping, as defined by the k_{off}, offered a robust and elegant displacement and purification method. Moreover, this new technology allows for mild purification conditions yielding a high recovery and purity within a fast, single purification step, even when starting from complex mixtures. Moreover, the method is applicable to very small sample sizes due to the high affinity and specificity of the applied binding agents.

The Examples provide support for the development, application and optimization of the NANEX technology, wherein several aspects have been highlighted. As mentioned herein, the binding agents throughout the majority of the example relate to ISVDs or Nanobodies, which are depicted herein by ‘CA’ numbering, relating to the sequence information for the specific Nb clones, as linked via the SEQ ID NOs provided herein. The Examples as given herein are not limiting and provide severable manners to enable the skilled person to apply the NANEX technology in a versatile manner.

As an overview, the Examples provide for a first proof of concept made by targeting a well-known and easily traceable target protein, Green fluorescent protein (GFP), wherein more specifically a subset of Nbs was generated for designing an optimal method. In particular, in Examples 1 and 2, based on the crystal structure of high (low picomolar) affinity Nb bound to GFP, a number of trapper/stripper pairing Nbs were designed by mutation of the paratopic residues, produced, purified and analysed by BLI to determine the kinetic constants. In Examples 1 to 6 these Nb binding agents targeting the same epitope on GFP with different kinetic constants were tested for their relative displacement potential in several combinations, which allows to conclude that binding pairs which target the same epitope of the target can be used for displacement, however with certain kinetic limitations, and mainly dictated by the relative difference in k_{off} value. Another way to generate a suitable trapper/stripper pair relates to increasing the avidity of the stripper as compared to the target. This has been shown in Example 12, where the stripper is a bivalent ISVD and the trapper a monovalent ISVD targeting the EPEA tag fused to a GFP protein.

Besides using Nb pairs which compete for the same epitope, Nanobody binding pairs which compete for a target kinetically but only partially (or not largely) overlapping epitopes have been demonstrated to also allow displacement, such as in Example 15 for factor IX.

In addition to purely Nbs, a functional variant of an ISVD, such as functionalized forms can as well be used as trapper or stripper agent, as is the case and shown herein for the Megabodies targeting GFP, usable as a stripper, as shown in Example 9, 10, and 16 or even as a trapper, as shown in Example 24.

Further trapper/stripper pairs were developed herein to target commercial tags as shown in Examples 1-10 and 18 for GFP, Example 12 for EPEA, Example 19 for GST, Example 20 for SMT3, and Example 21 and 22 for mCherry. Besides designing and generating pairs based on structural information of the binder/target complex, the Examples 21 and 22 also demonstrate that more straightforward tools are available to obtain suitable pairs.

Moreover, target proteins do not require a tag for NANEX, but may also be recognized at native or specific endogenously displayed epitopes, as has been shown in Example 13 for Synaptojanin, Examples 14, 15, 16, and 23 and 24 for factor IX.

Another application has been demonstrated in the field of protein-protein interactions (PPI), since purification using NANEX also allows to identify and purify PPI complexes. This was shown for eGFP-GR (Glucocorticoid receptor) (Example 25) and eGFP-ARb (Androgen receptor) (Example 26) which were purified with a GFP trapper/stripper pair and revealed co-purification of the HSP proteins known to form a complex with these nuclear receptors.

With regards to the possibilities to design kits and products for applying NANEX, we demonstrated several ways of immobilizing the trapper, as to further apply the stripper in solution. For instance, in Examples 1 to 5, the trapper was immobilized using agarose beads, in Example 27 magnetic beads were used. Furthermore in for instance examples 6 to 10 the immobilization of trapper Nb was obtained by coupling to a resin and packing in a column. To miniaturize the system, in Example 11, a microcolumn was applied, and in Example 28, a microchip prototype has been tested.

Finally, the diversity of samples that may be used herein for purification is clear from the examples since for instance bacterial lysates (e.g. in Example 1 and further), yeast extracts (e.g. in Example 17, 27), human plasma (in Example 23 and 24), and HEK cell lysates (in Example 25, 26), all complex mixtures, provide for one-step purification options using NANEX. Moreover, where a second purification step is desired, a tandem purification using NANEX sequentially and/or using multiple trapper/stripper pairs is in place as well (e.g. Example 16).

### Example 1. Purification of GFP Protein Spiked in a Bacterial Lysate by NANEX Chromatography, Using Nb CA15816 as an Immobilized Trapper on HiTrap NHS-Activated Sepharose HP Columns and Nb CA12760 as a Stripper

To proof the concept that proteins can be purified by Nanobody exchange chromatography (NANEX), we selected two Nanobodies that bind substantially the same or largely overlapping epitope on GFP and used these Nanobodies to purify GFP protein according to FIG. 1.

First, we selected a high affinity Nanobody against GFP following standard procedures (Pardon et al., 2014) to be used as a stripper. CA12760 was selected as a Nanobody that binds GFP proteins with low picomolar affinity. Bio-Layer Interferometry (BLI) assays identified the affinity (K_{D}) to be 40 pM, kₒₙ 2.56×10⁵ (M⁻¹.s⁻¹) and k_{off} 0.032×10⁻³ (s⁻¹). To perform NANEX chromatography, we next designed a nanobody that binds substantially the same or largely overlapping epitope on GFP to be immobilized on a solid phase as the trapper by site directed mutagenesis. Maximal elution could be obtained when using

NANEX with an immobilized trapper Nanobody that has a lower affinity and/or higher off-rate compared to the stripper Nanobody that is used to competitively elute the target, which vice versa has a higher affinity and or lower off-rate for the target protein. Based on the structure of the GFP⋅CA12760 protein complex (FIG. 3, Example 2), we designed CA15816 as an engineered version of CA12760 with a lower affinity and a higher off-rate. Indeed, CA15816 was designed by mutation of two residues contained in the paratope (T54A, V55A) to decrease its affinity compared to CA12760.

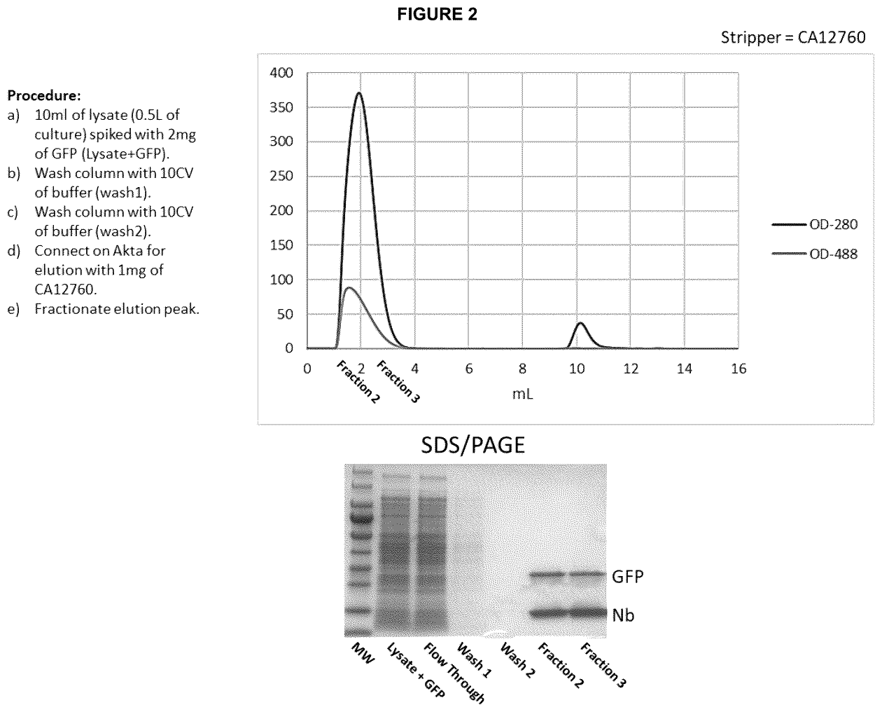

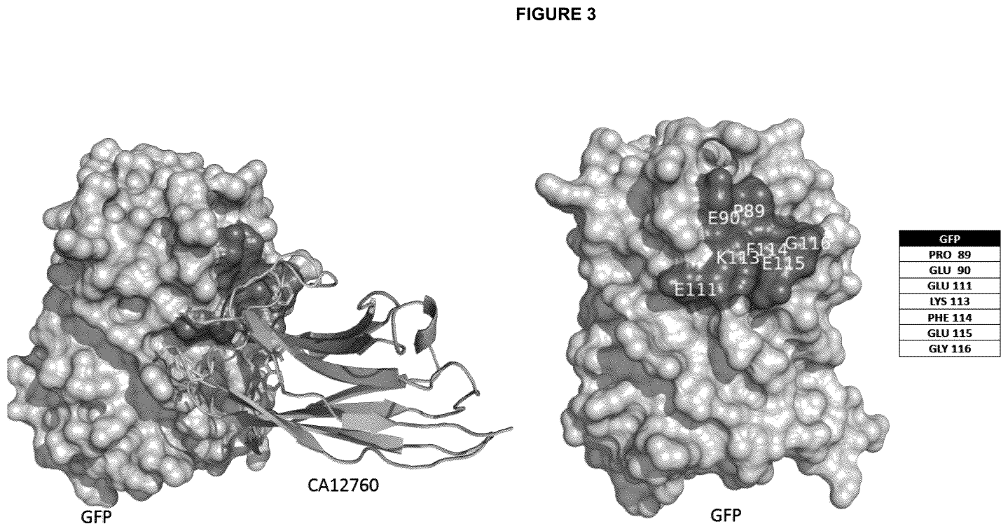

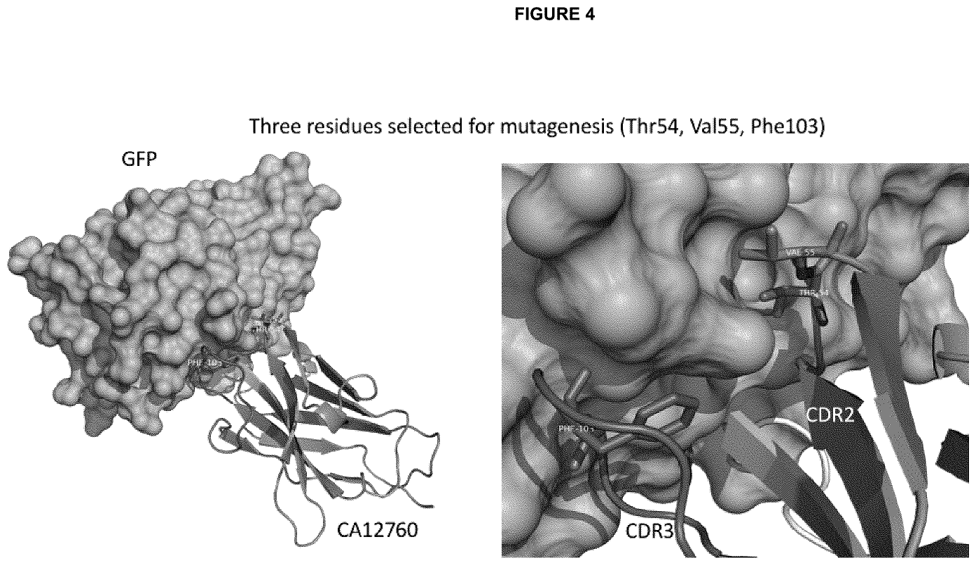

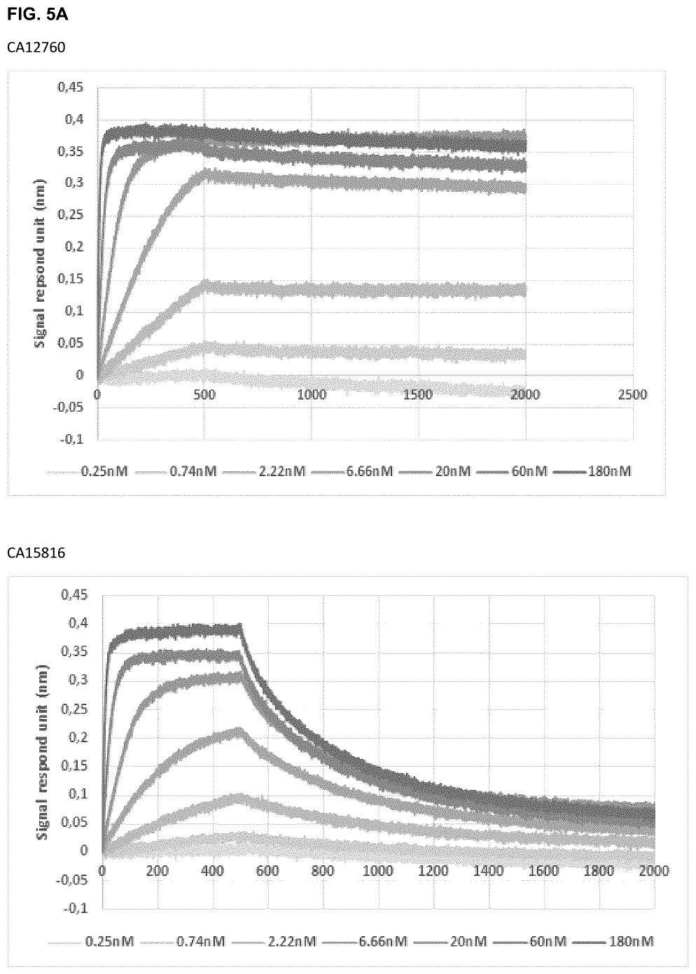

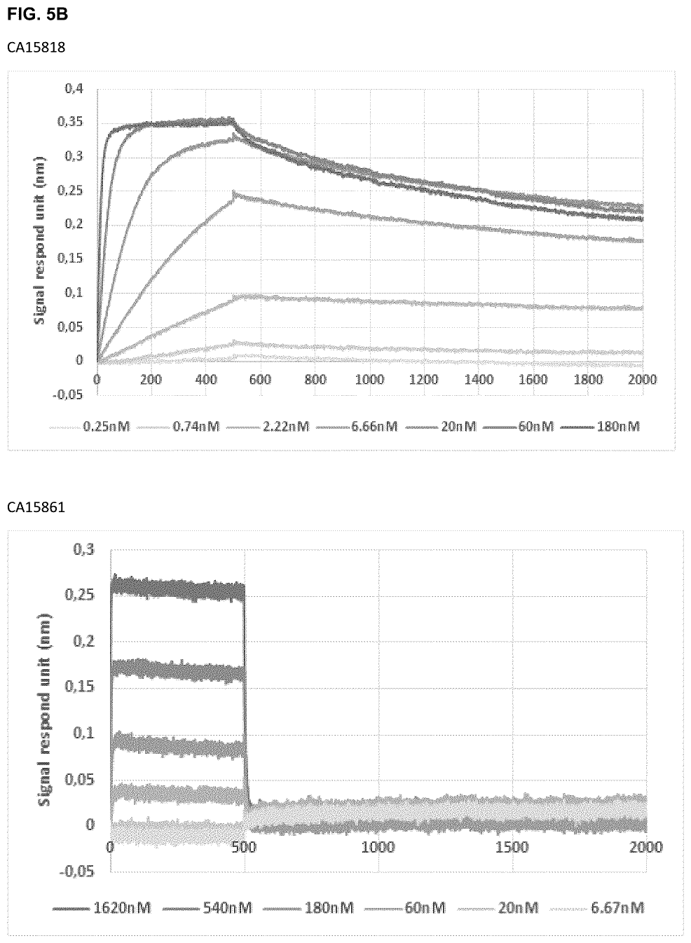

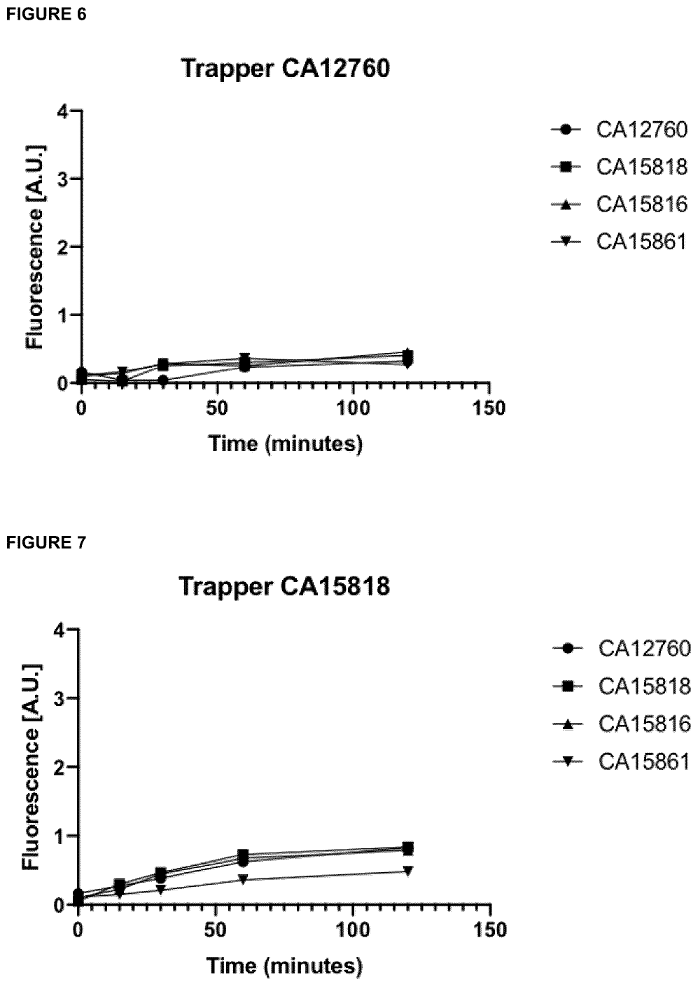

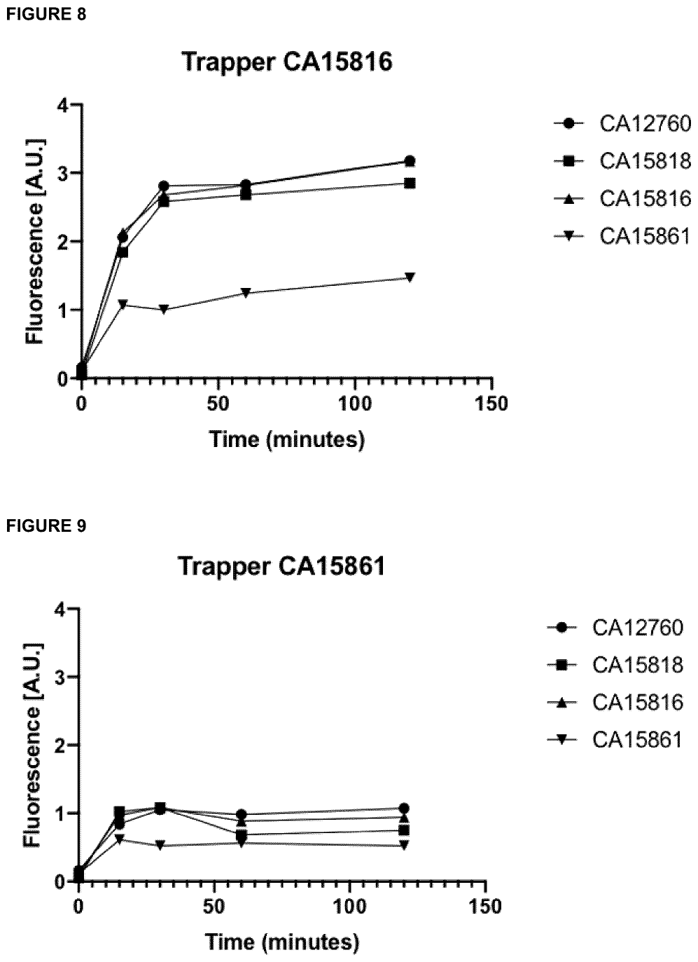

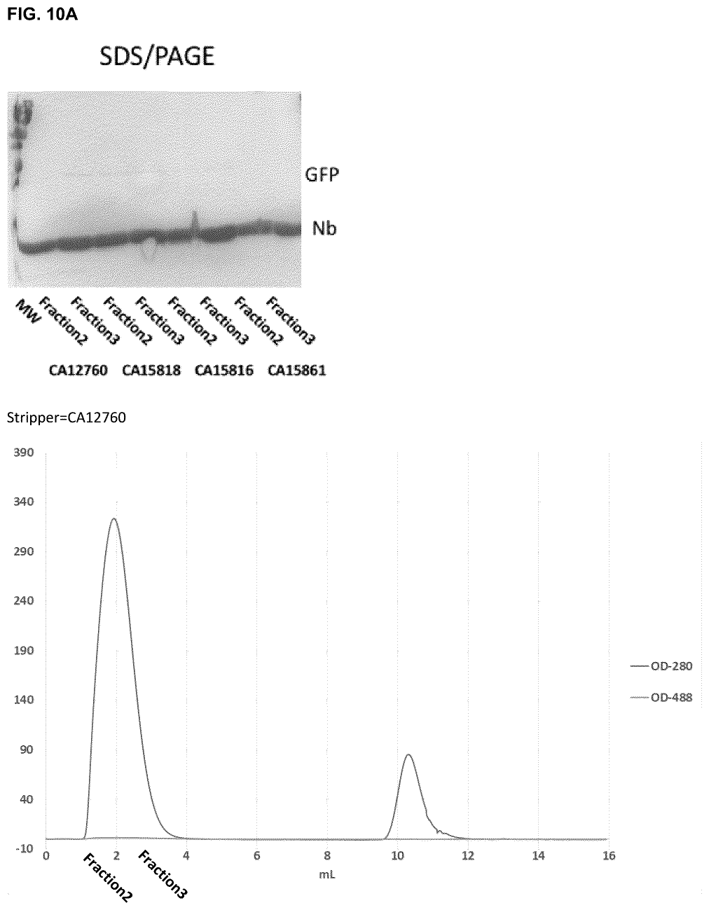

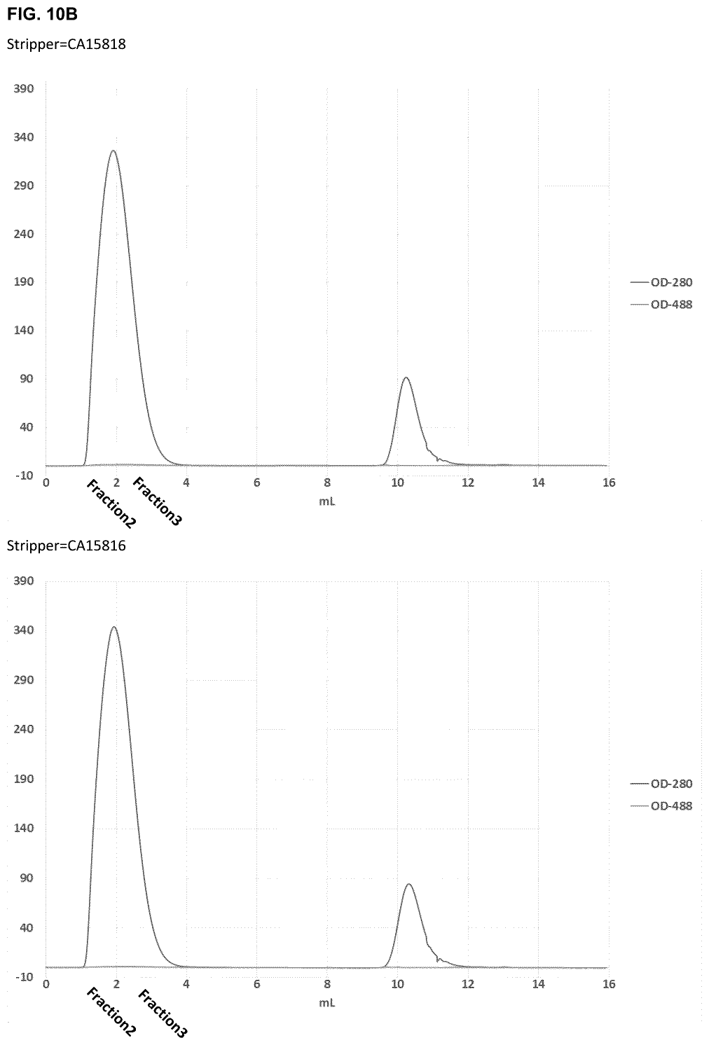

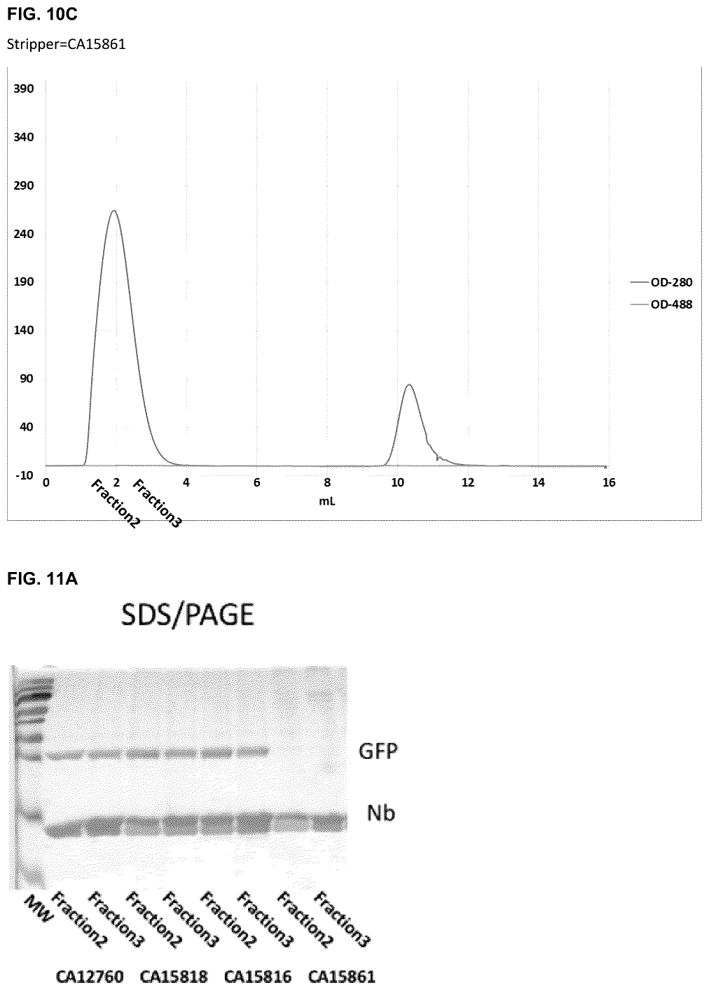

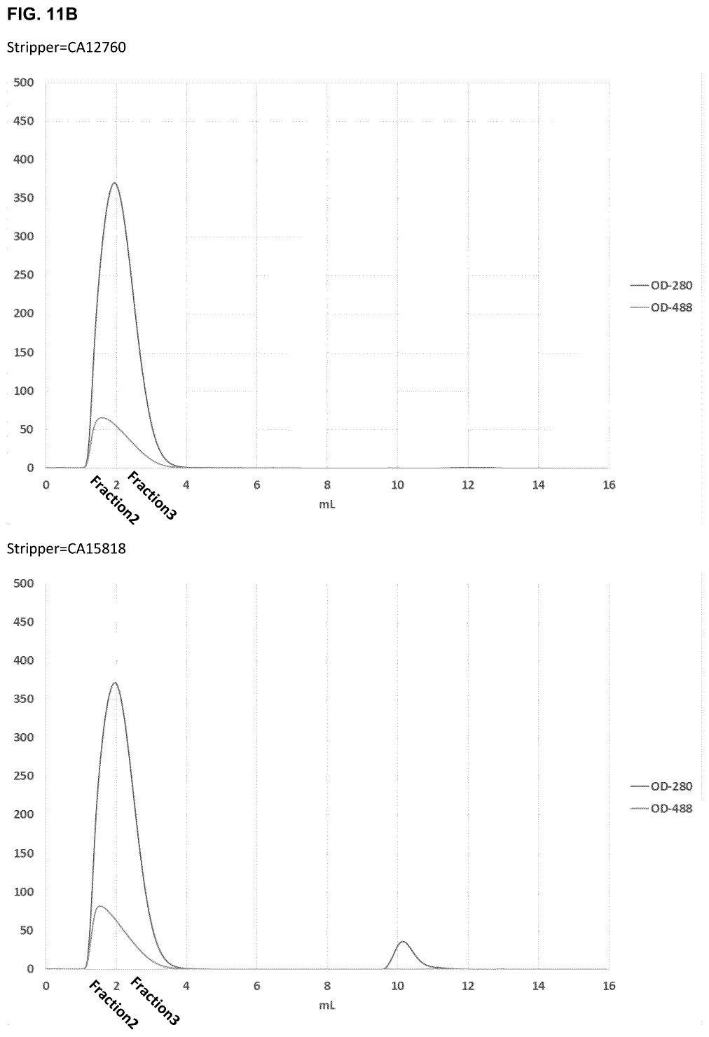

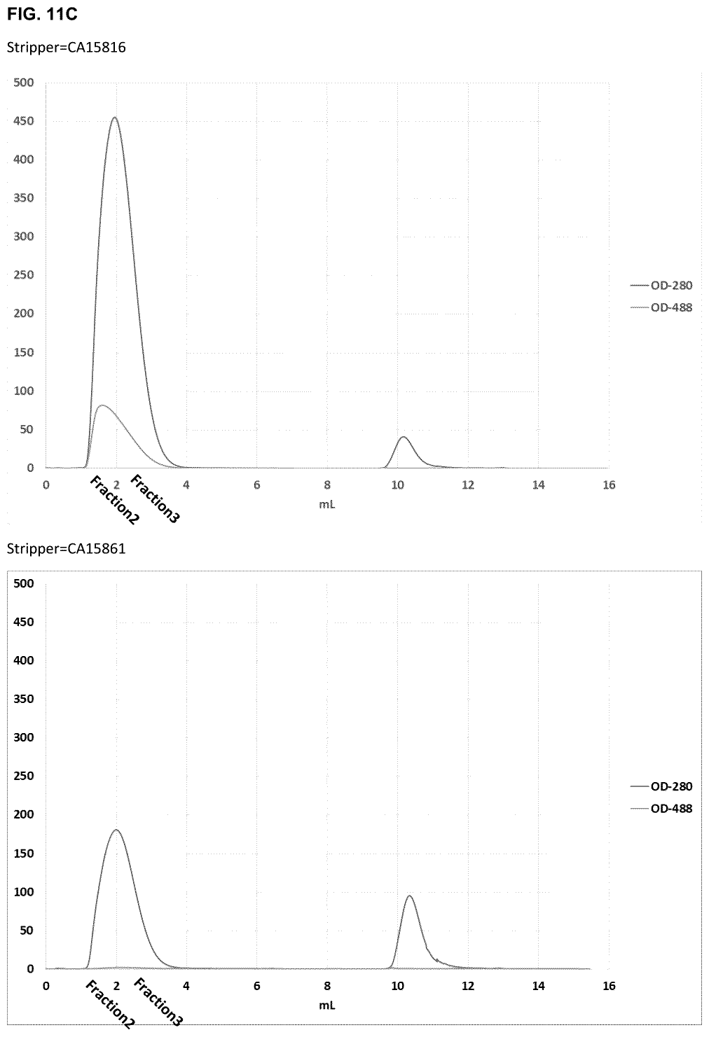

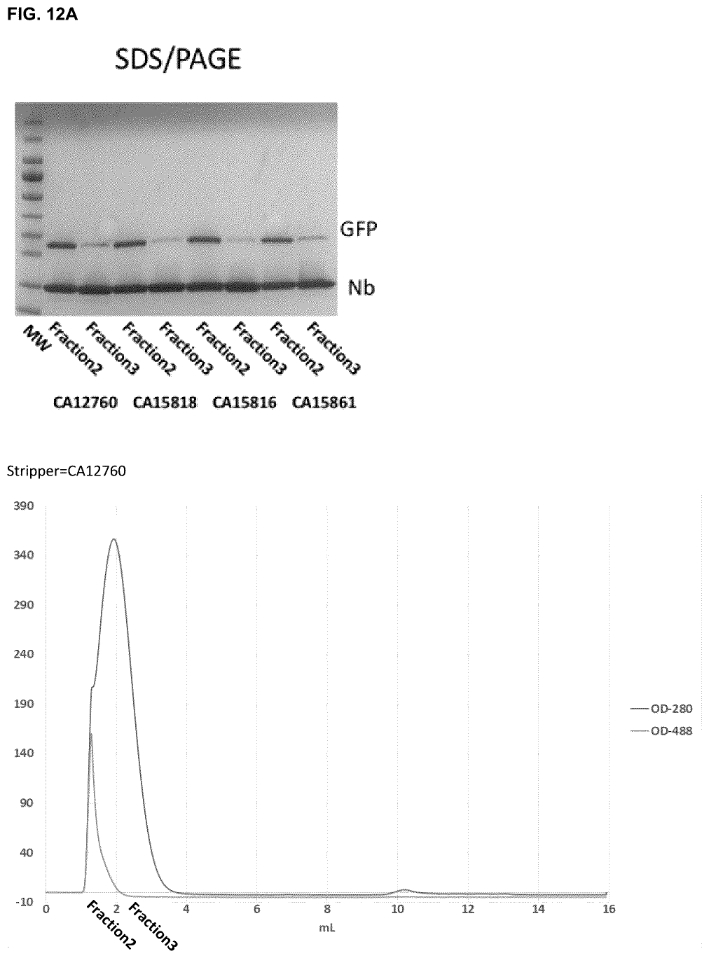

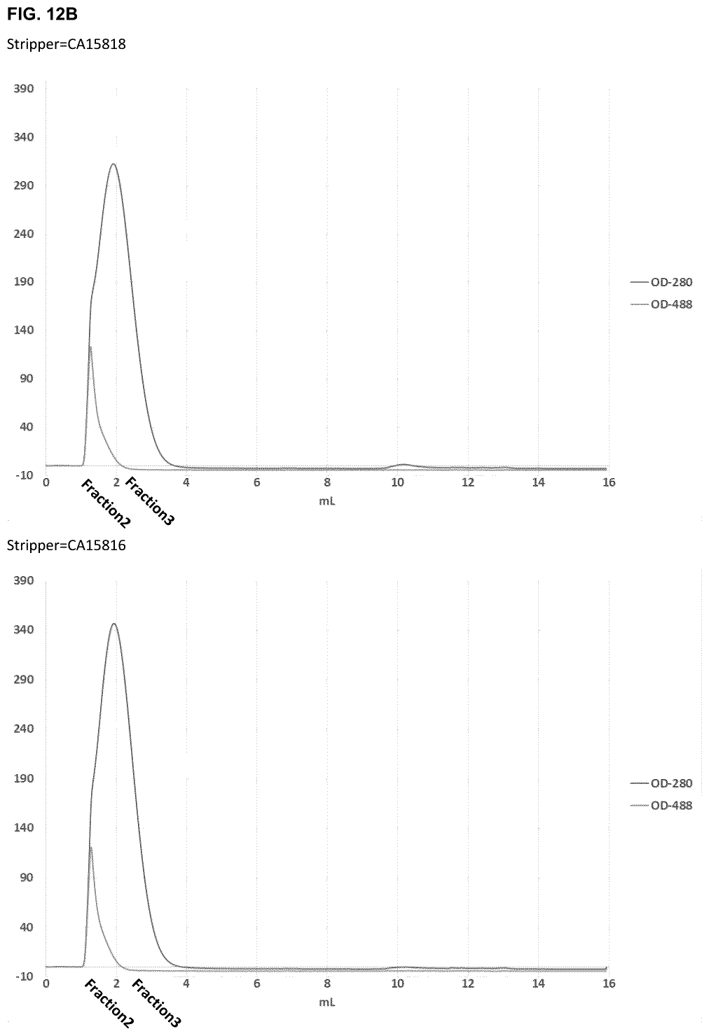

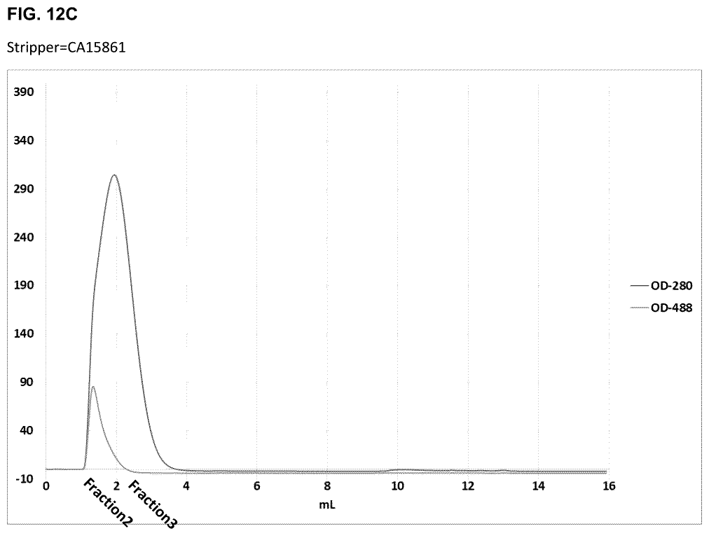

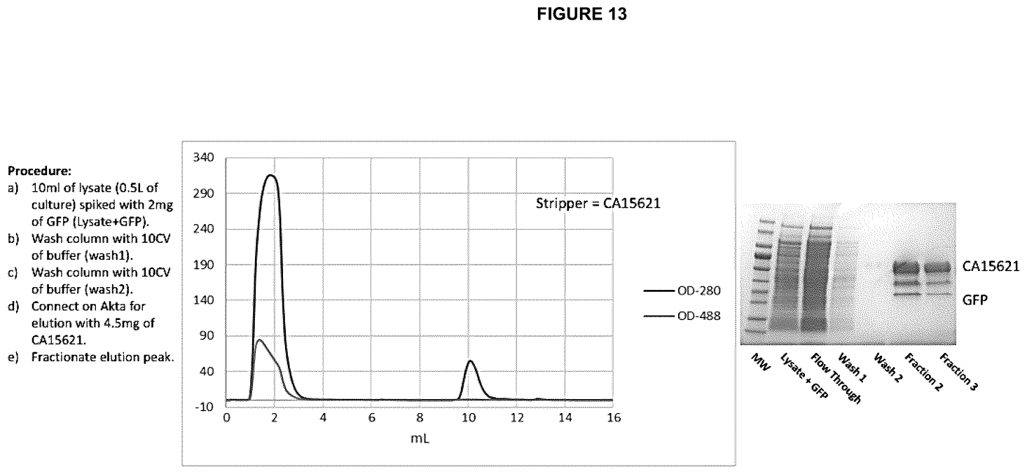

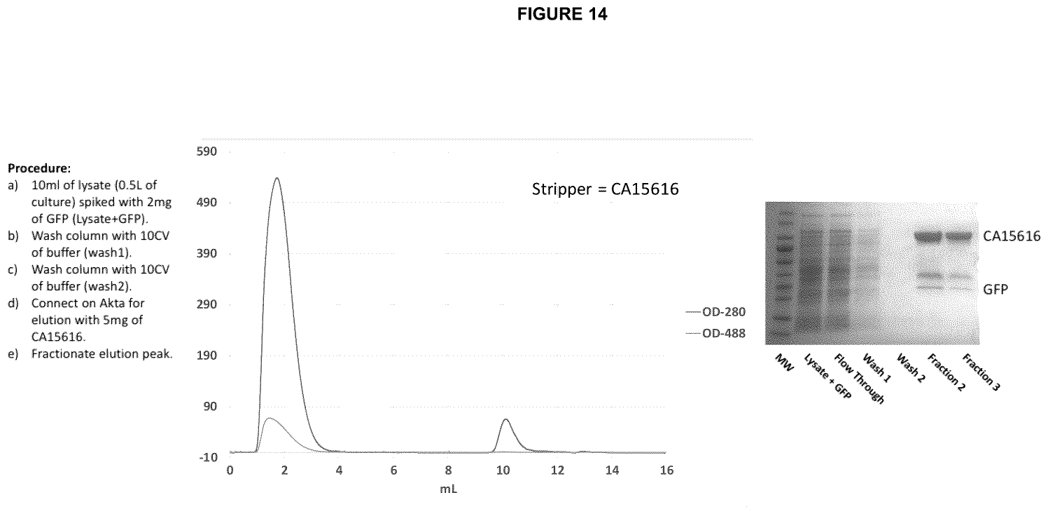

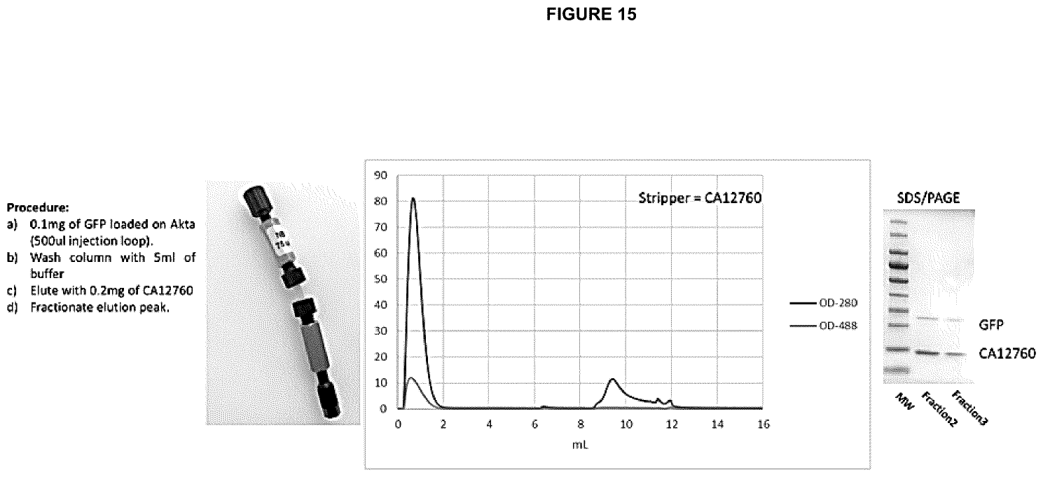

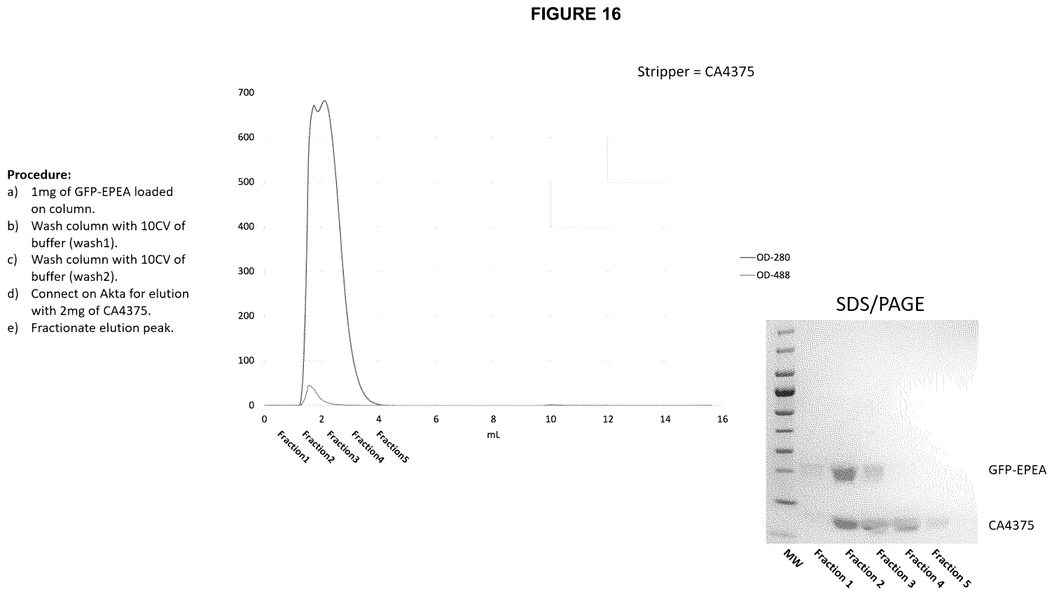

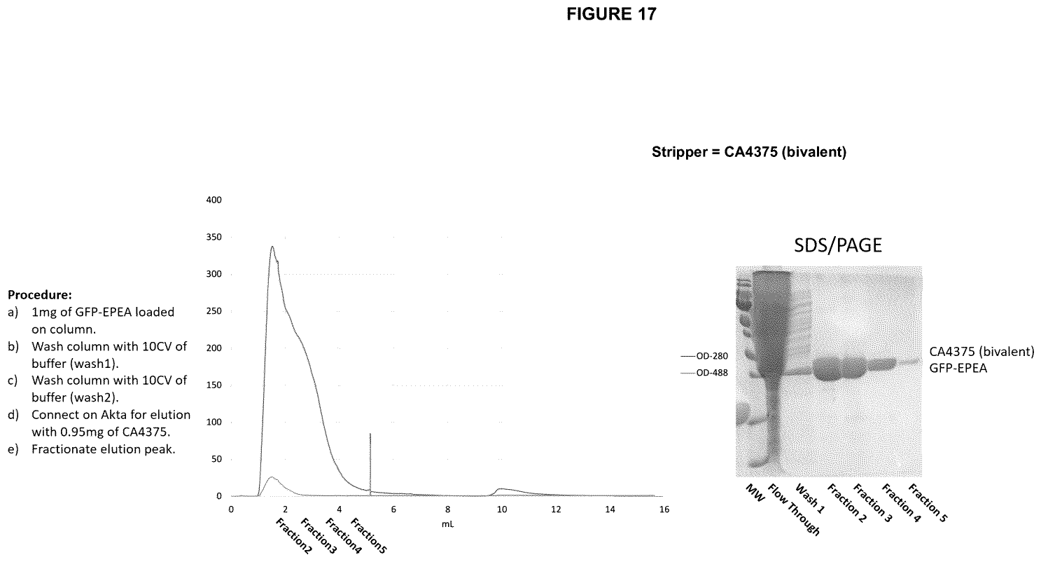

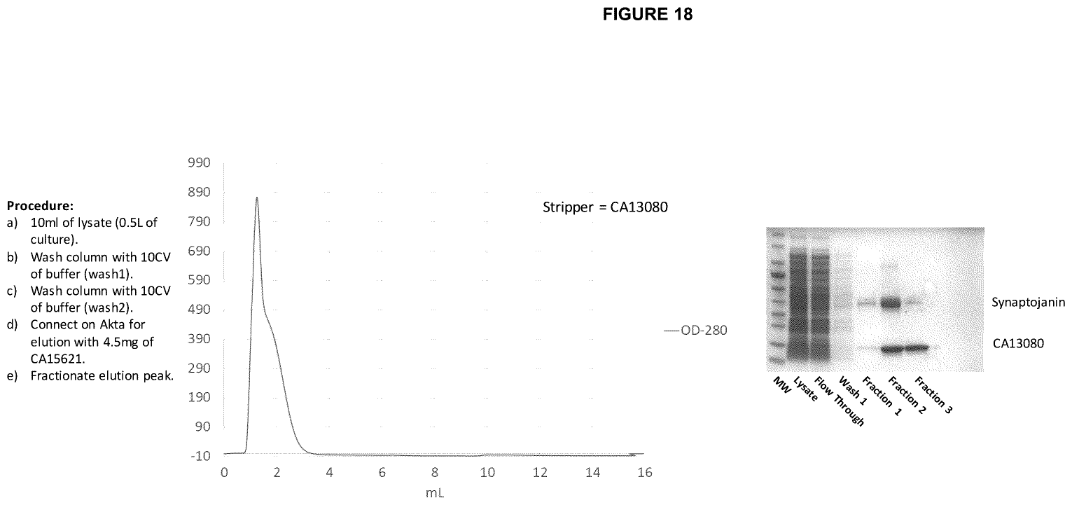

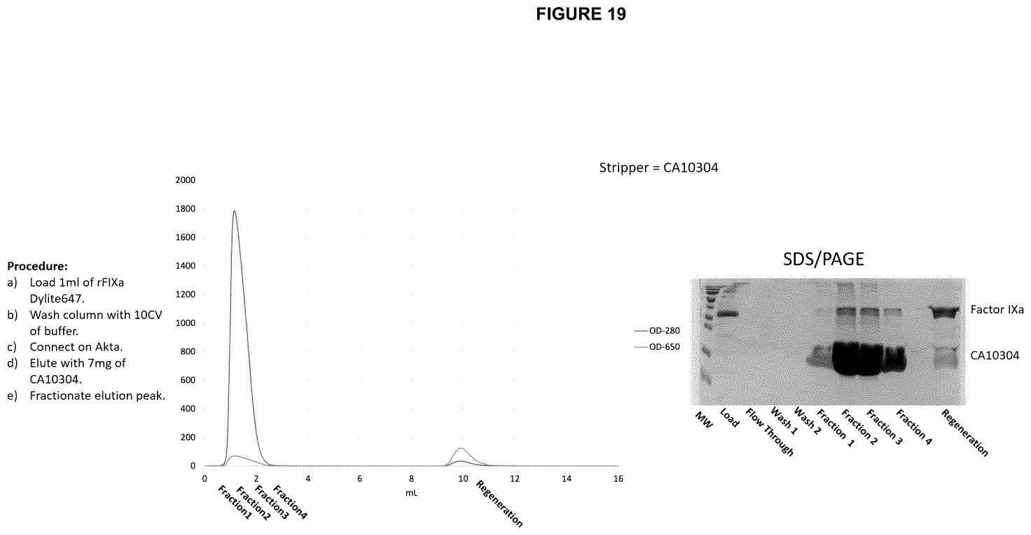

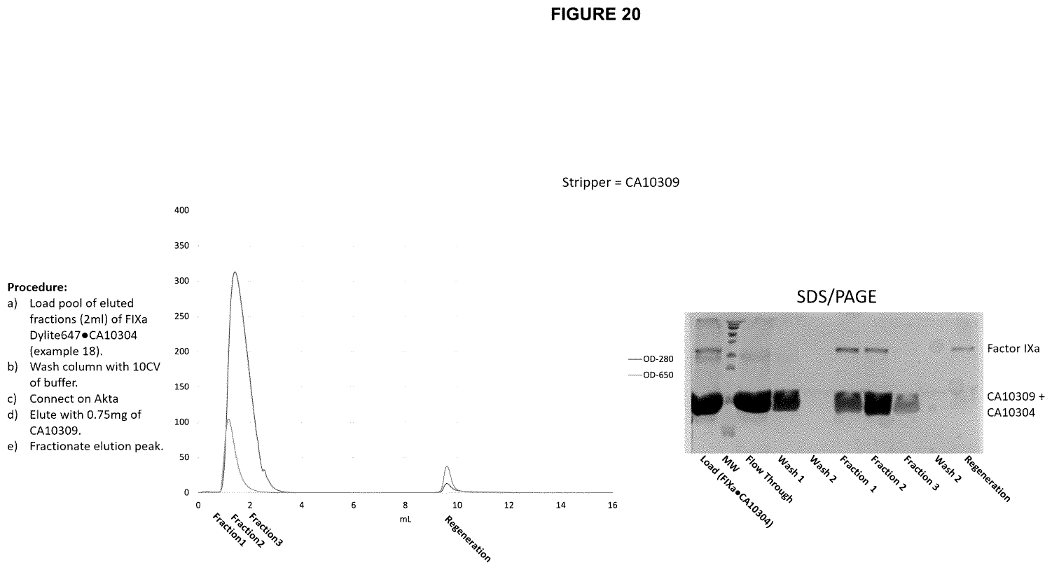

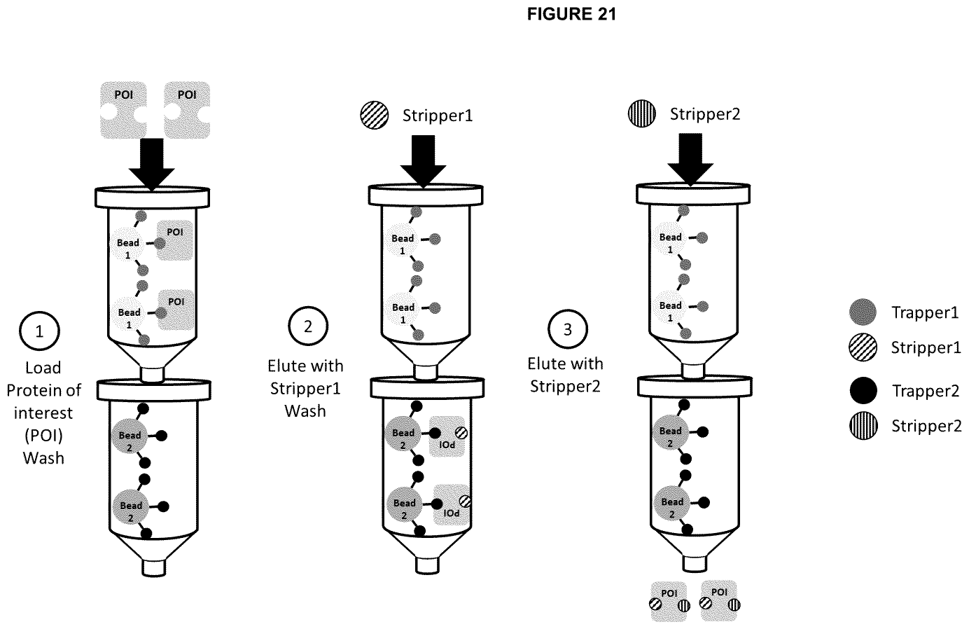

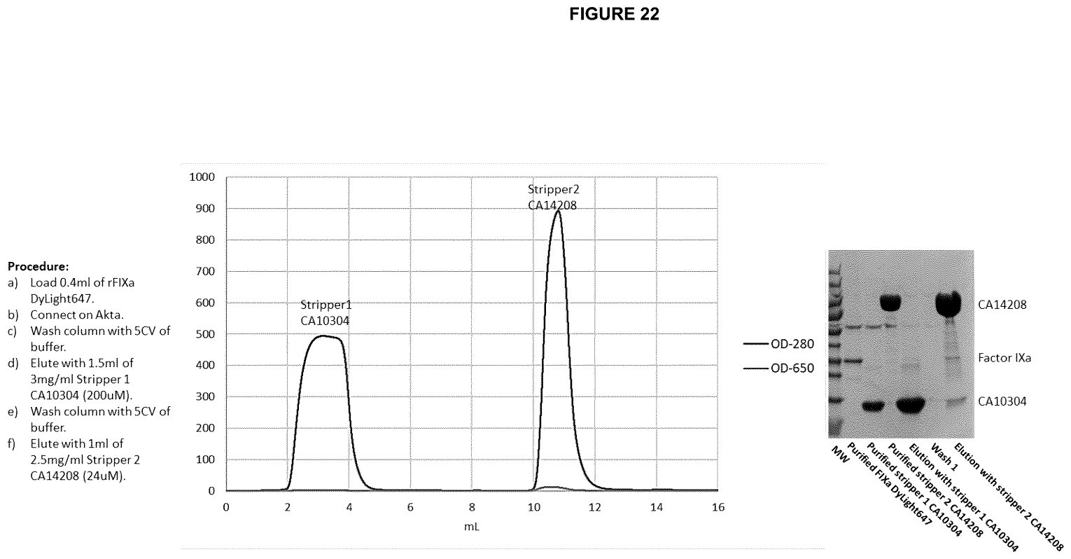

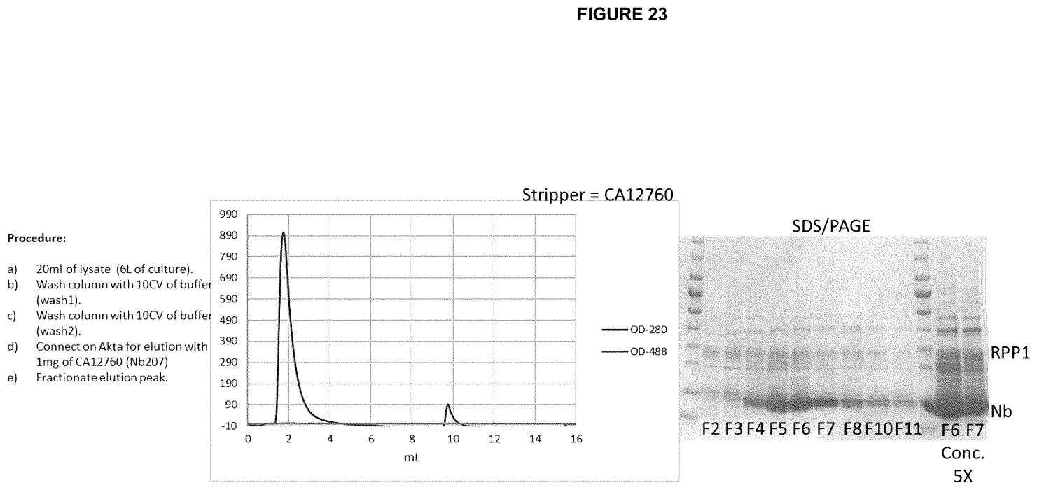

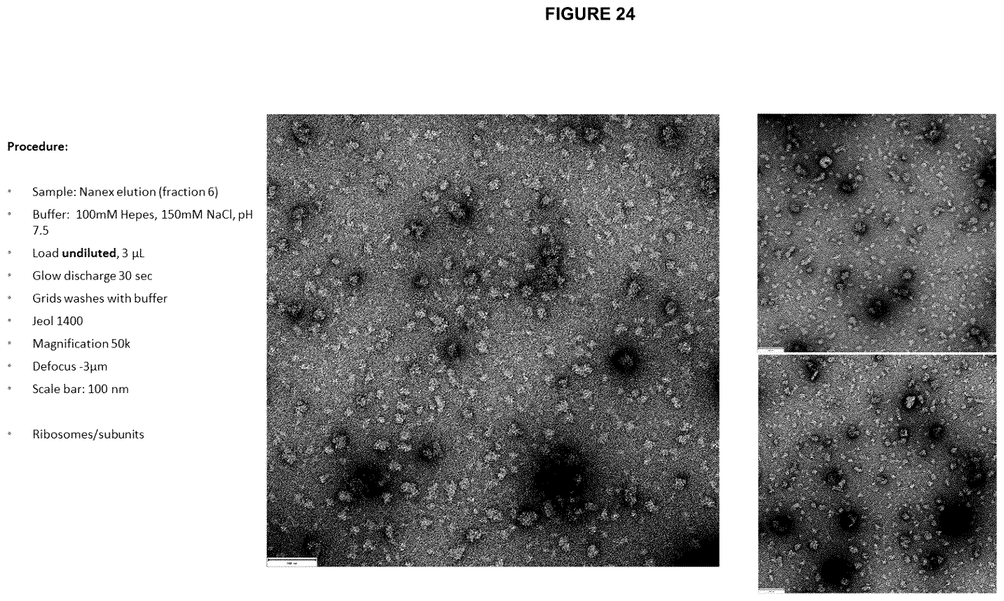

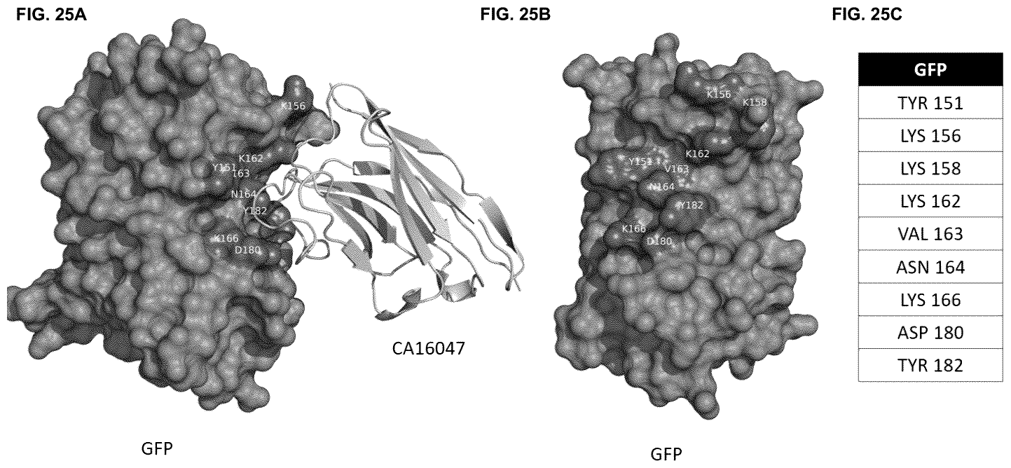

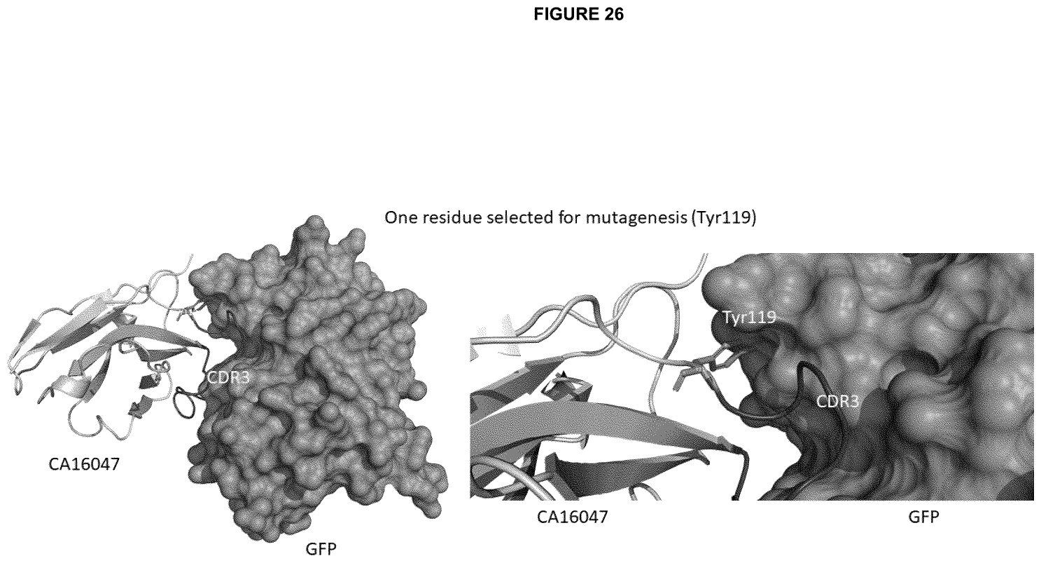

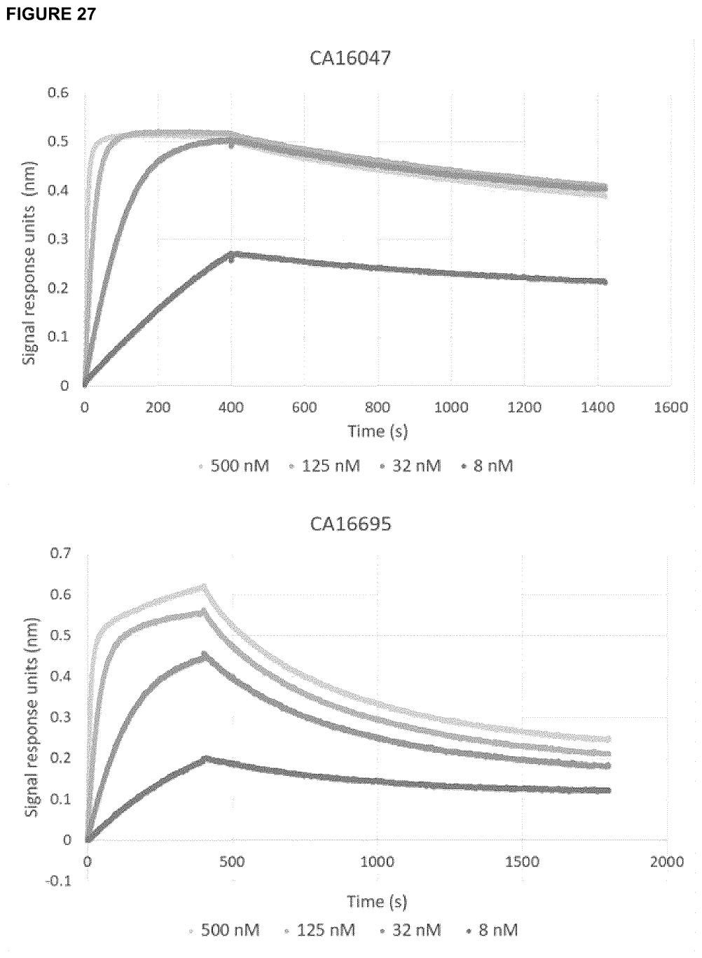

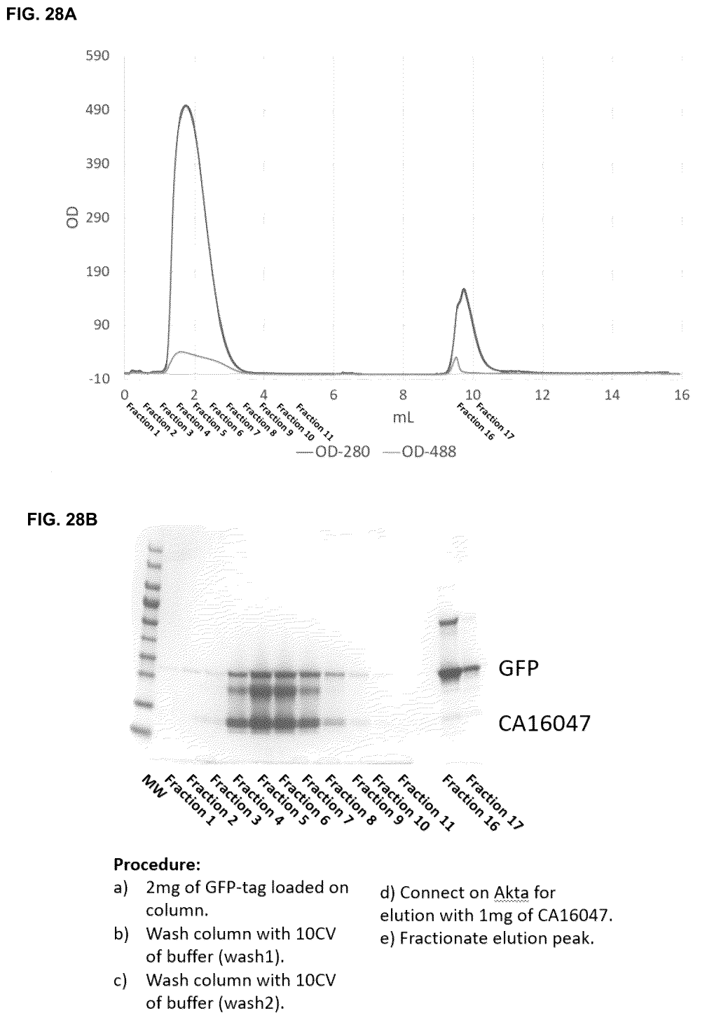

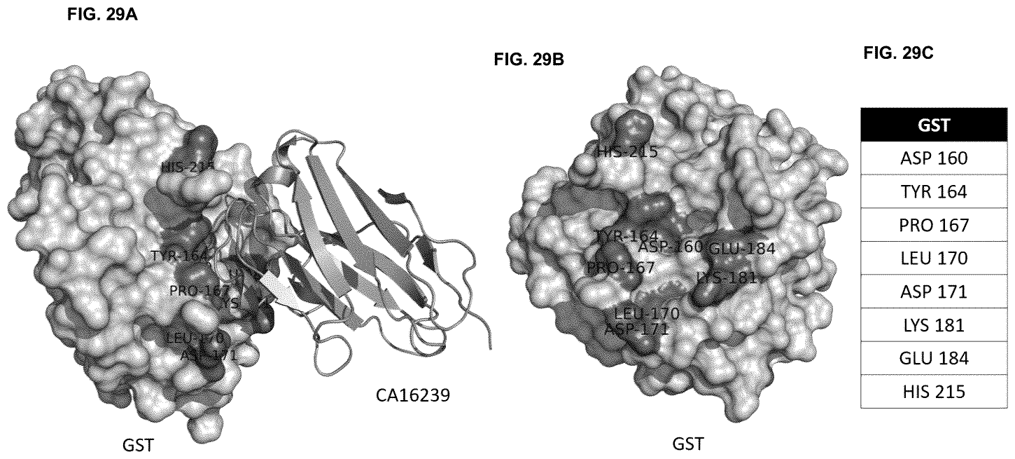

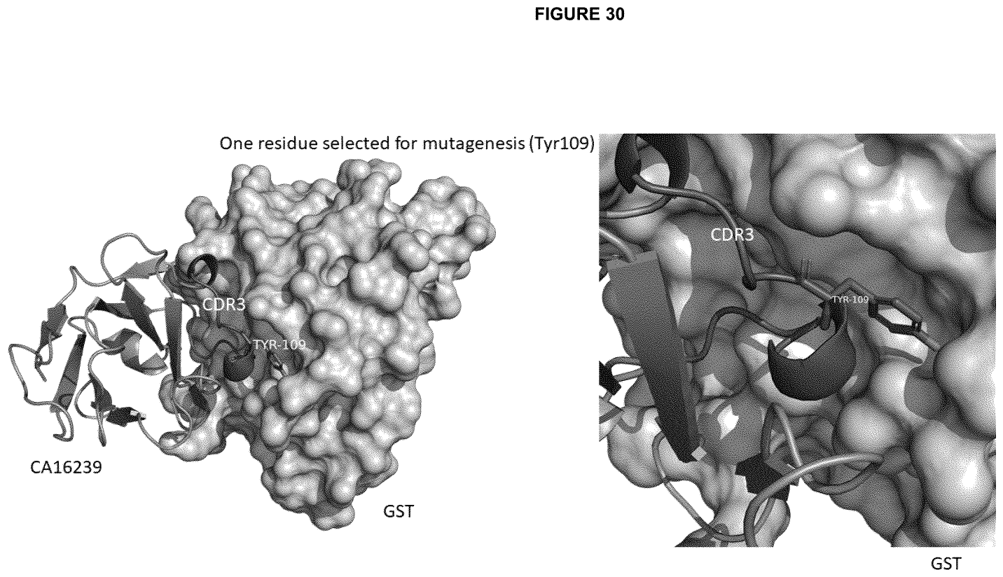

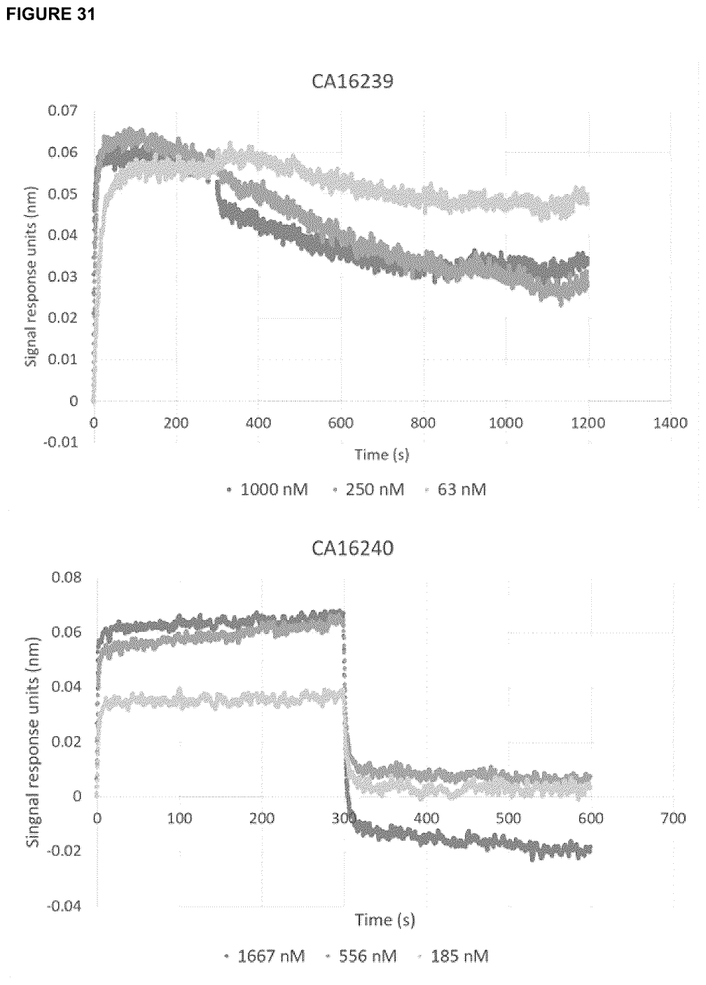

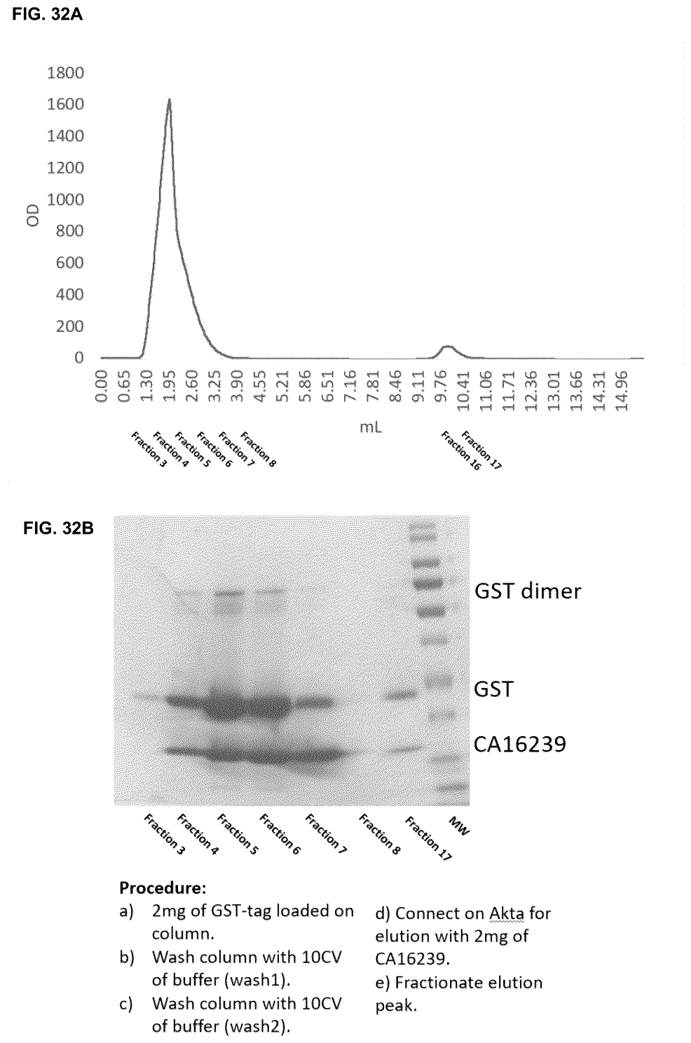

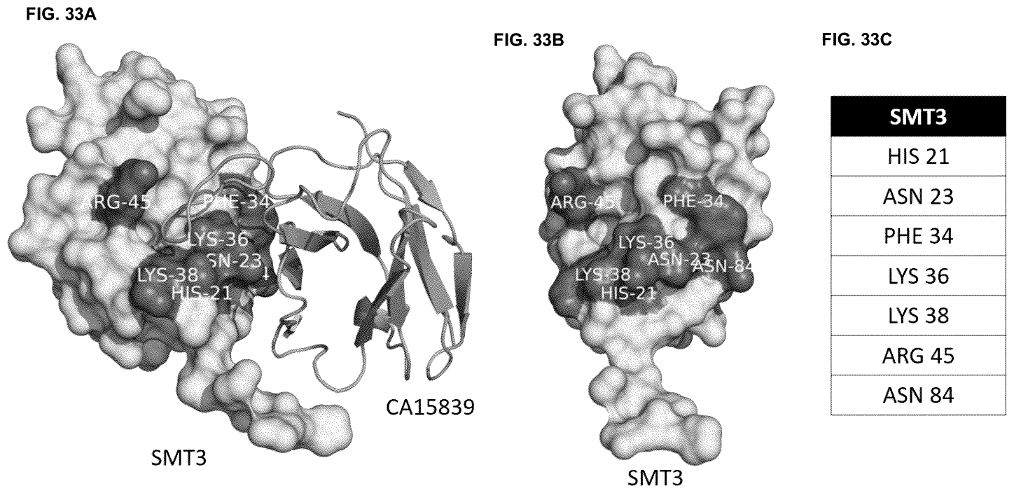

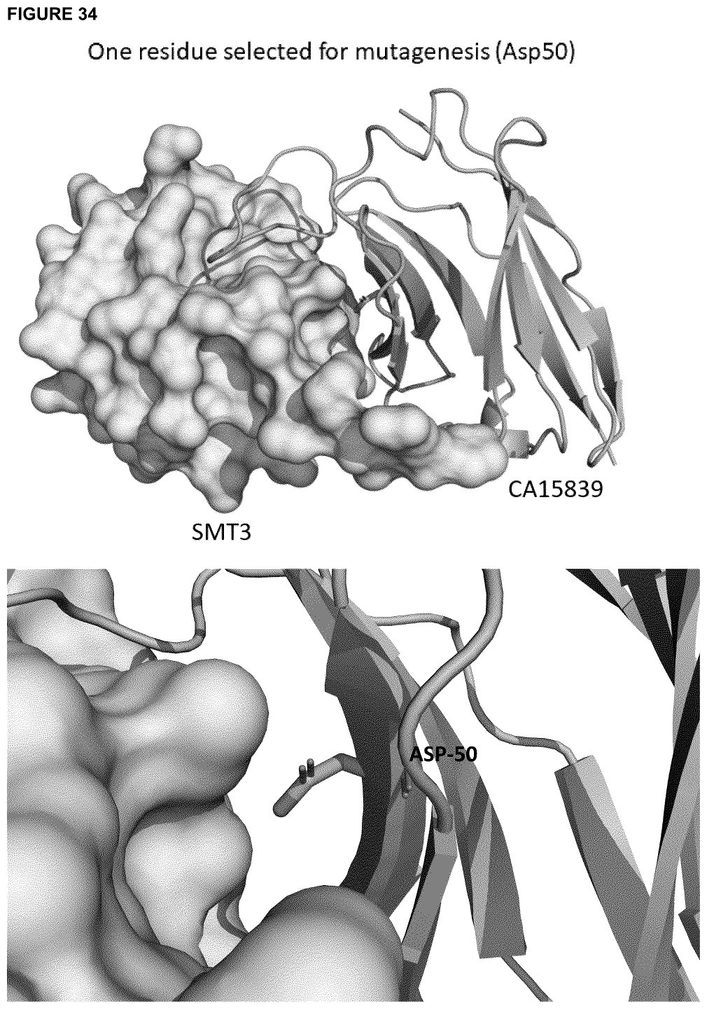

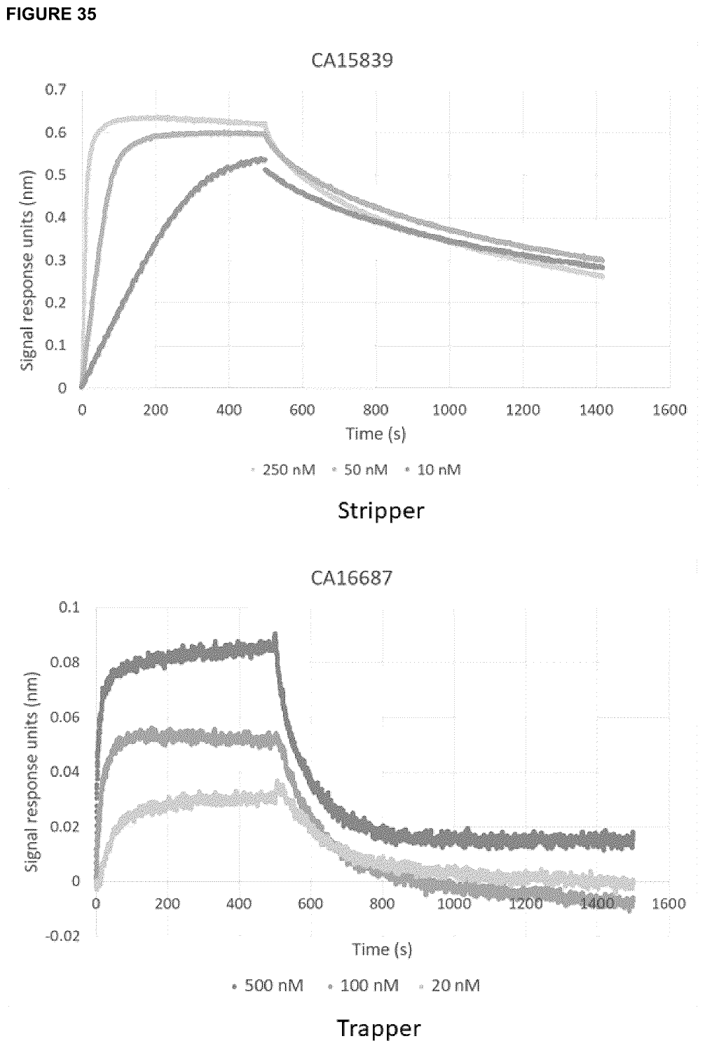

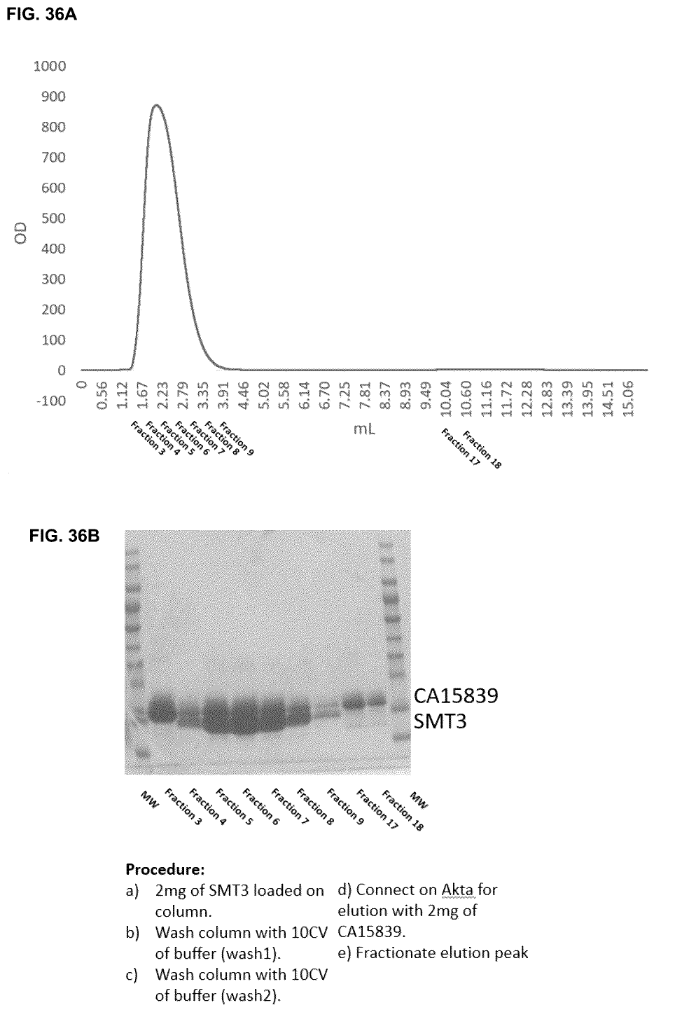

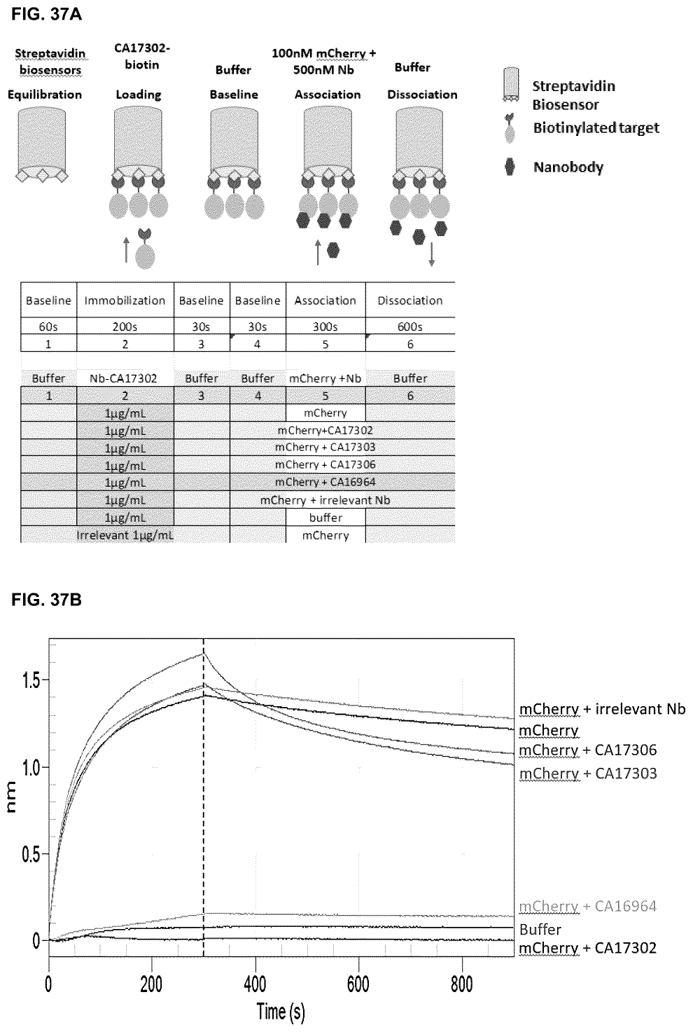

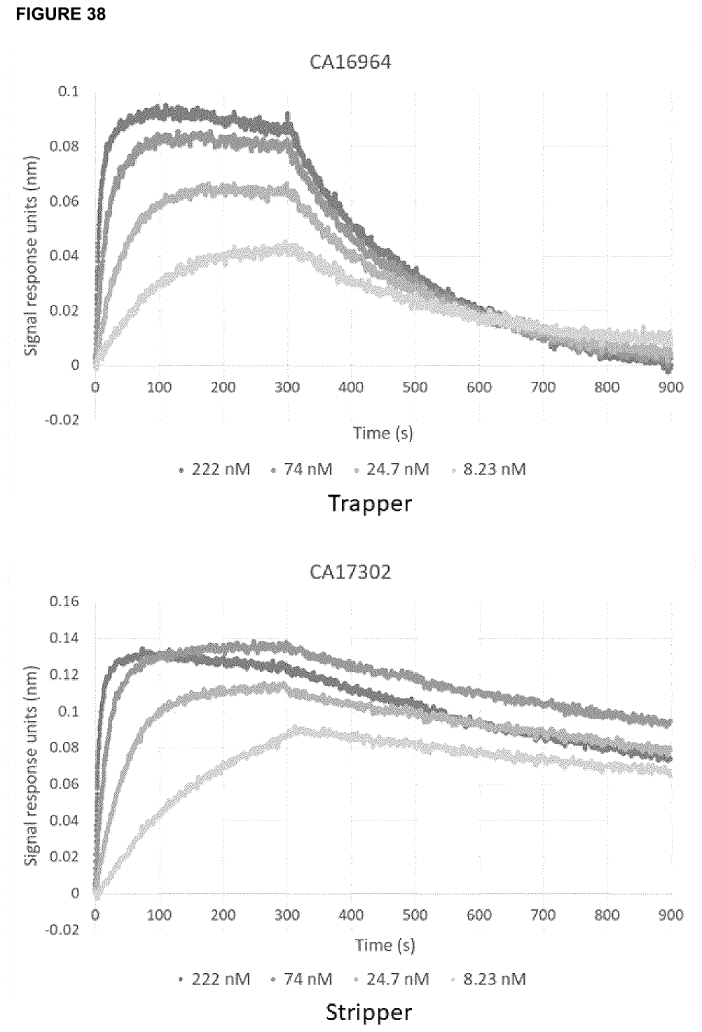

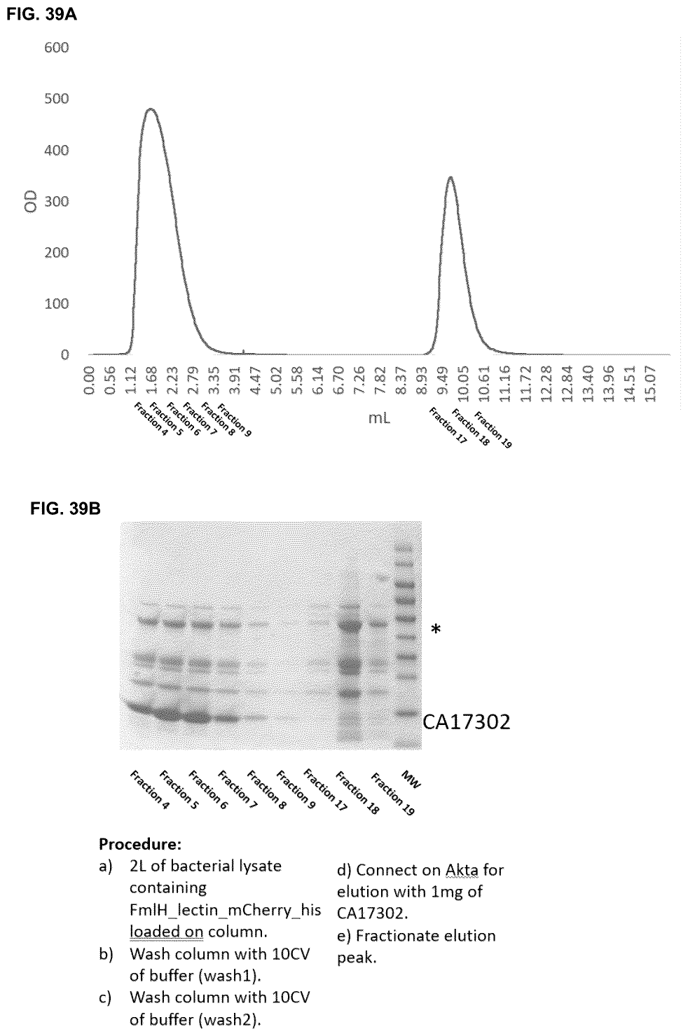

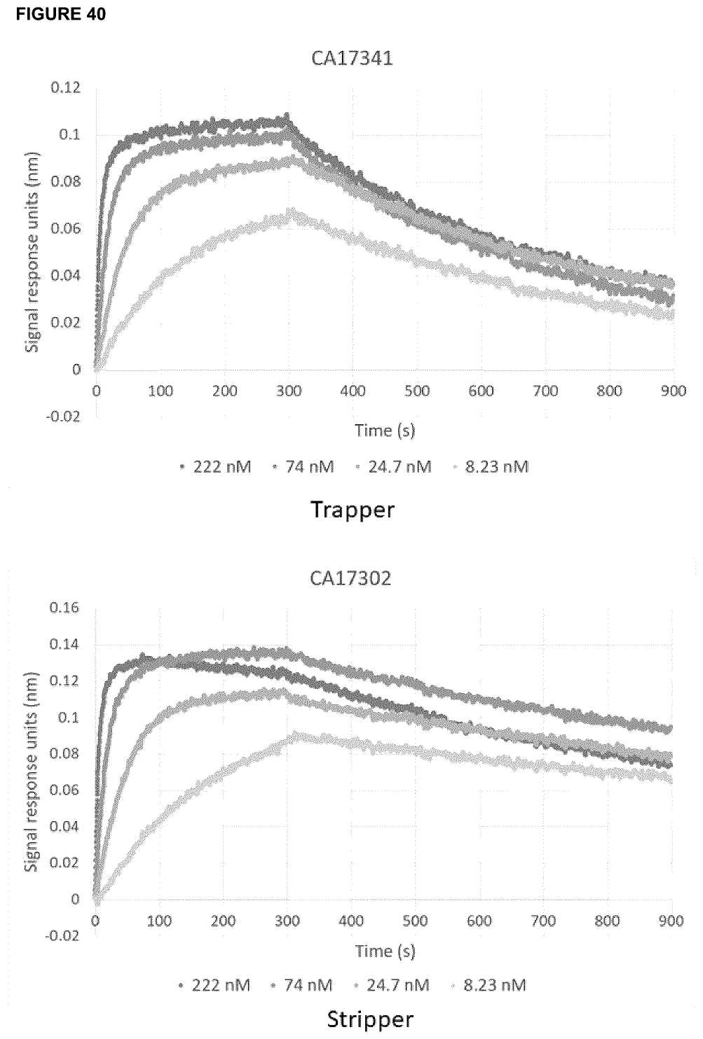

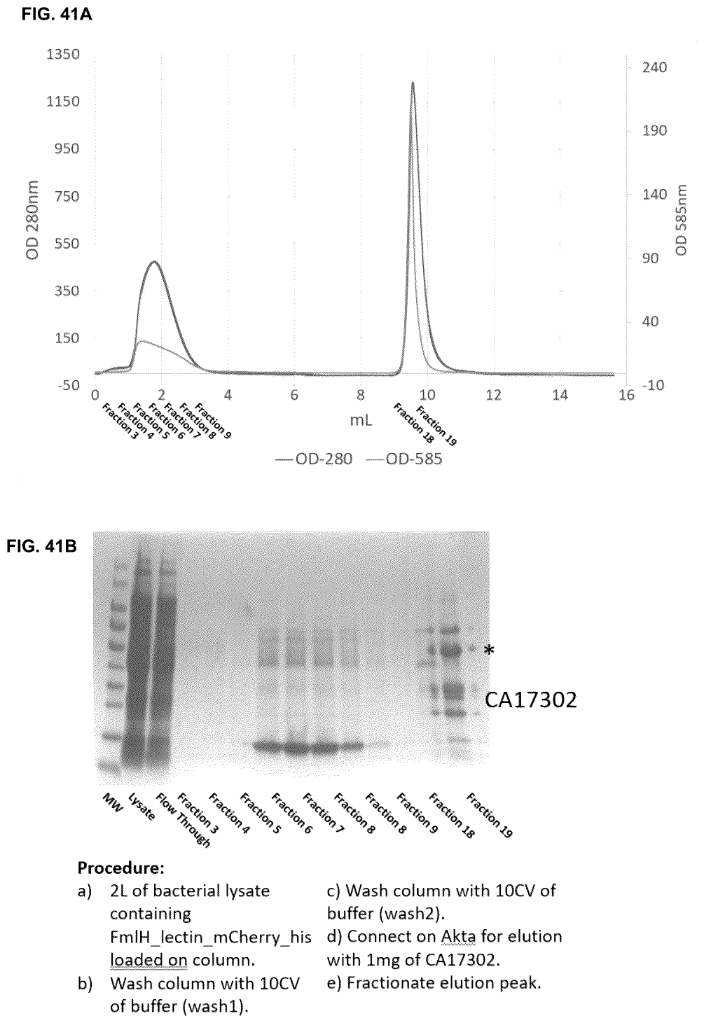

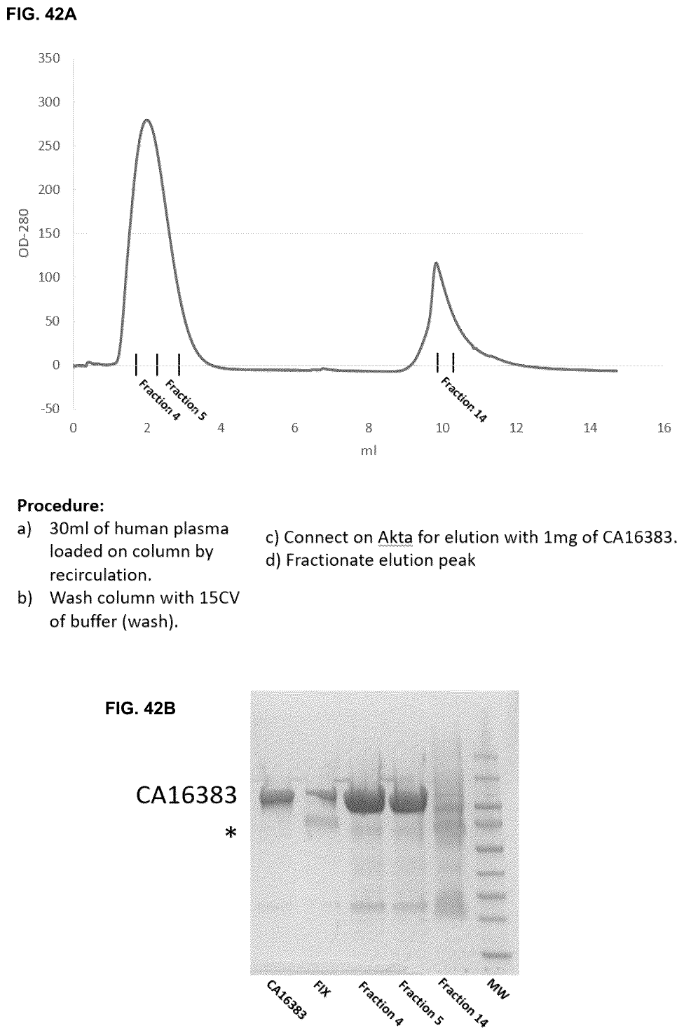

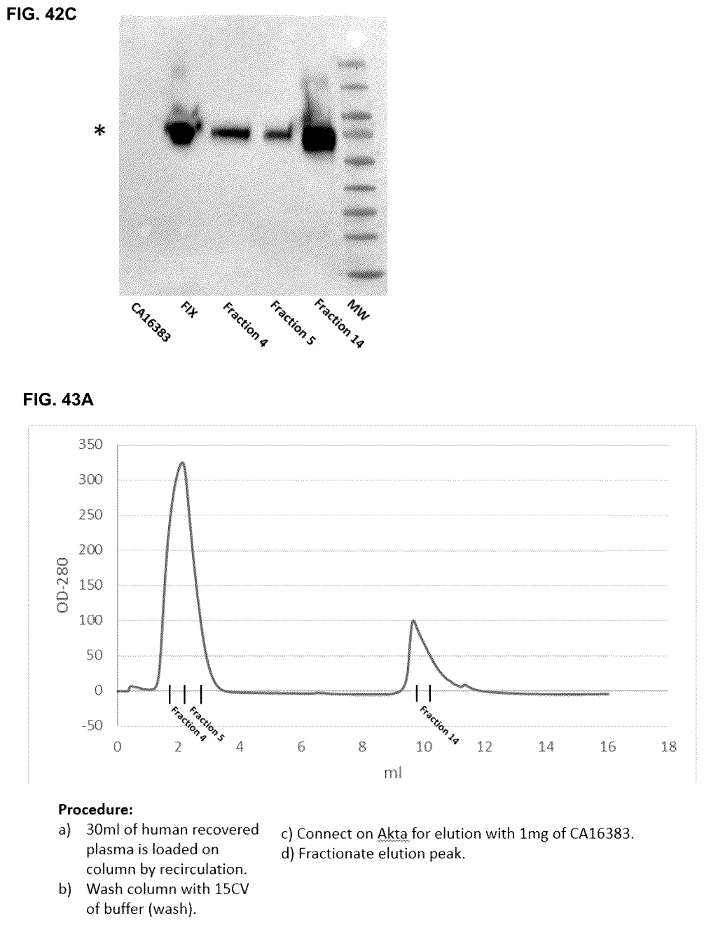

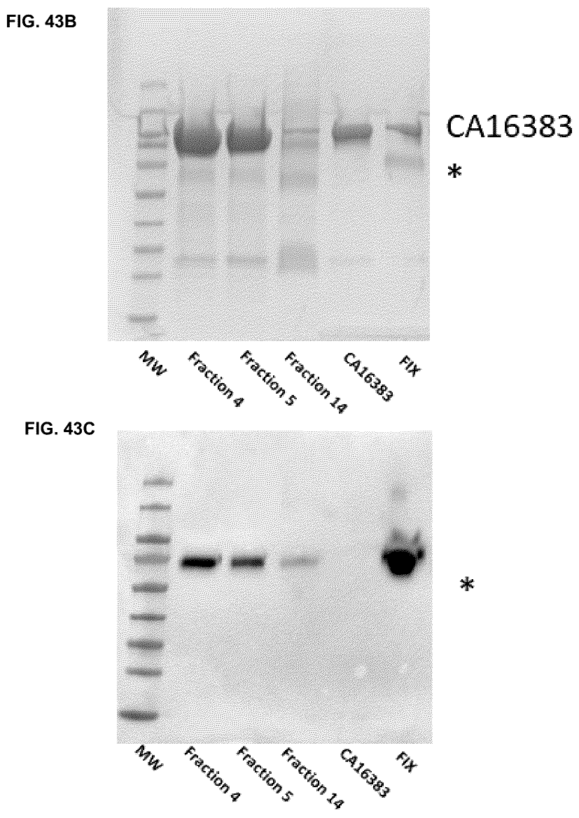

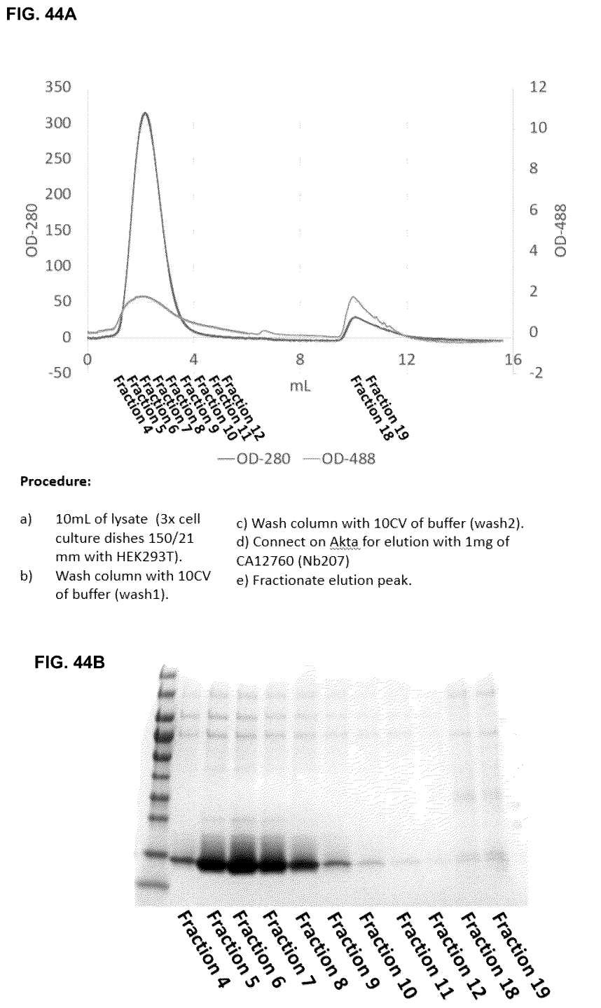

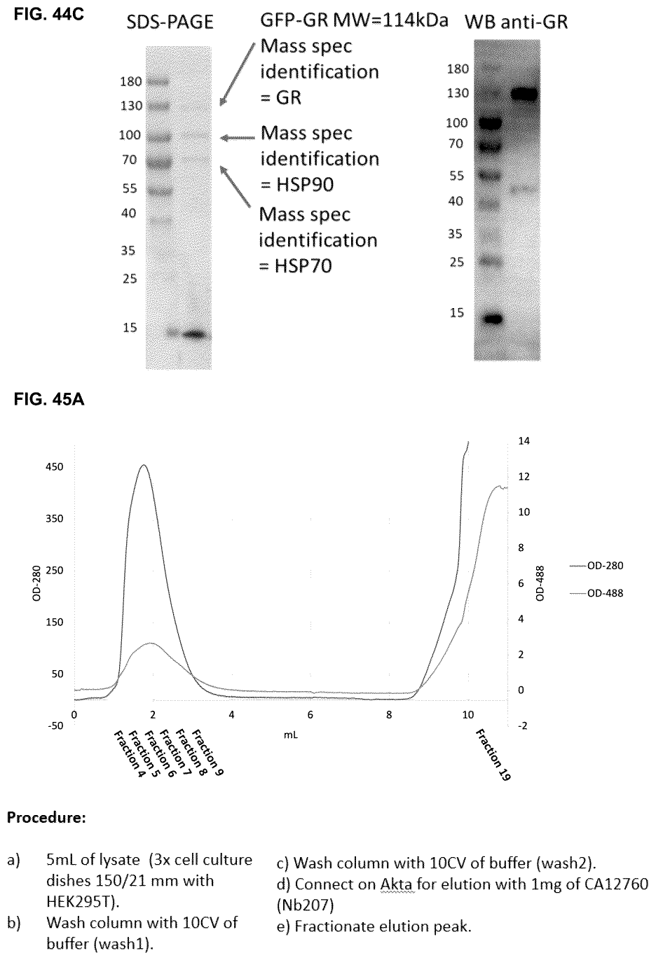

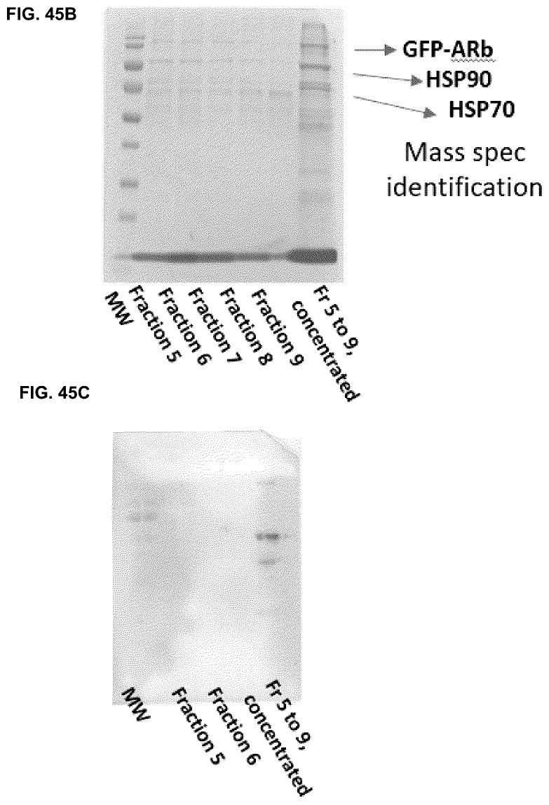

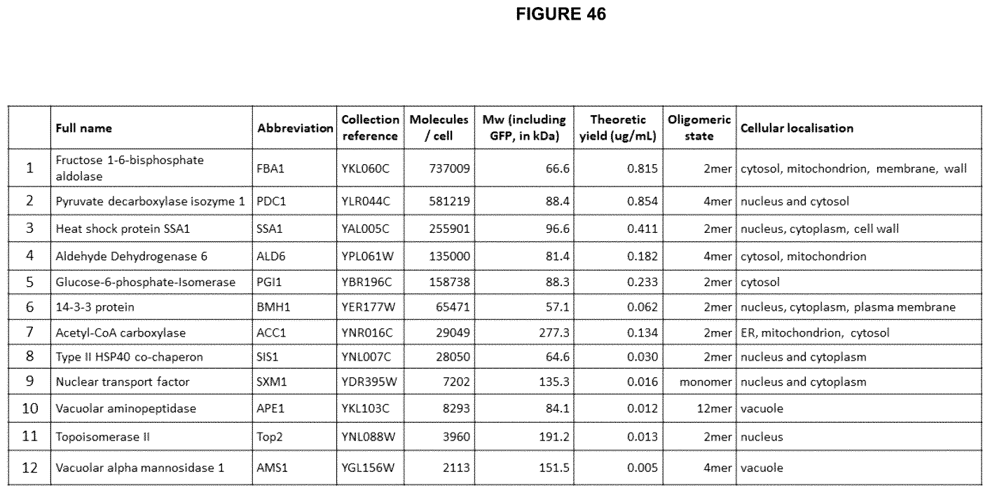

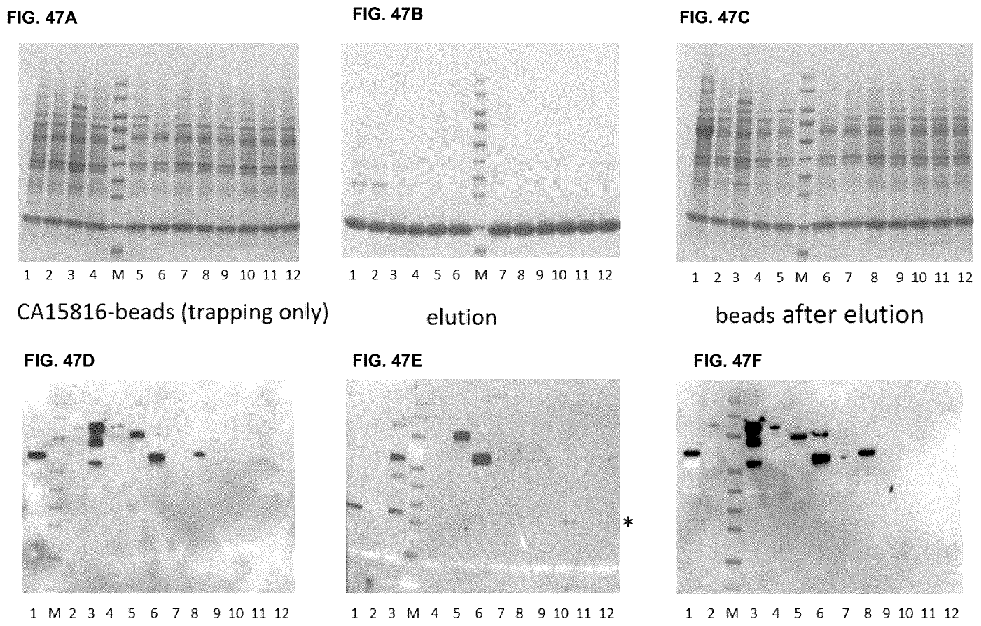

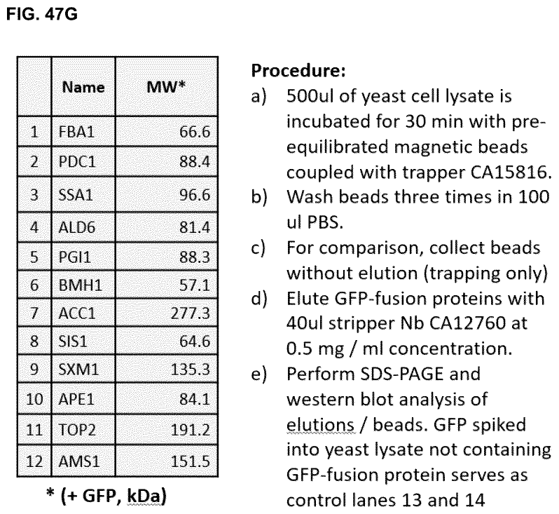

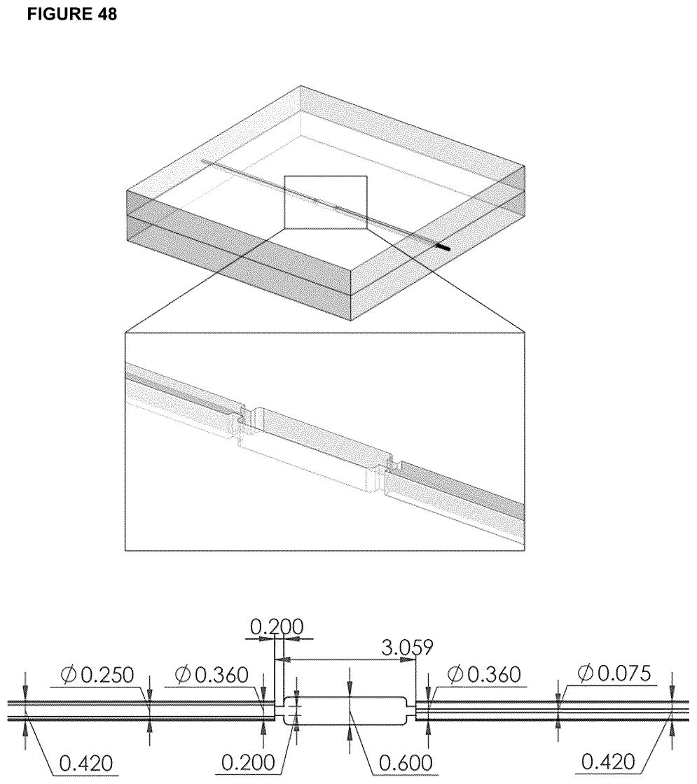

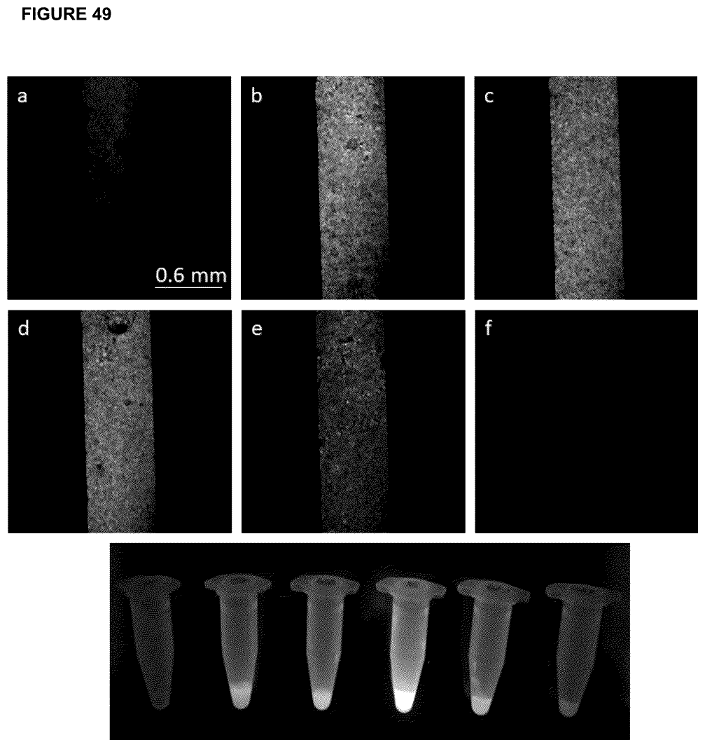

BLI assays identified the affinity (K_{D}) of CA15816 for the GFP epitope to be 2.7 nM, kₒₙ 8.35×10⁵ (M⁻¹.s⁻¹) and k_{off} 2.25×10⁻³ (s⁻¹). One mg (66.67 nmol) of CA15816 Nb was immobilized on HiTrap NHS-activated Sepharose HP column (GE) following the supplier's recommendations. Ten mL of a bacterial lysate (equivalent to the cell pellet of 0.5 L of an E. coli culture grown in LB) was spiked with 2 mg of GFP. The spiked lysate was loaded on the CA15816 column using a syringe, washed twice with 10 mL (10 Column volumes (CV)) of buffer (100 mM Hepes pH 7.5, 150 mM NaCl). The column was then connected on the Akta pure FPLC system (GE) for elution using the stripper in 8 CV of elution buffer (100 mM Hepes pH 7.5, 150 mM NaCl, 66.67 μM stripper Nb (1 mg/mL)) at a flow rate of 0.1 mL/min. The elution was collected in 500 μl fractions and the major elution peak was analysed by SDS/PAGE gel (FIG. 2). Regeneration of the column was obtained by 8 CV of 200 mM Glycine buffer at pH2.3.

The purification of GFP protein using CA15816 as an immobilized trapper on a HiTrap NHS-activated Sepharose HP column that was eluted from this column by CA12760 as a stripper was monitored by measuring the absorbance at 280 nm (Protein absorption) and 488 nm (GFP fluorophore absorption). From the elution profile, we can conclude that the washing was sufficient for removing most of the impurities, and that the elution fractions 2 and 3 contained the Stripper Nb and GFP protein as detected in the main peak. The smaller peak at 280 nm represents the remaining sample (or GFP protein) that was washed off from the column with the regeneration buffer.

Nanobody exchange chromatography (NANEX) using binders for the same epitope requires that the immobilized Nanobody (trapper) has a higher off-rate and/or lower affinity, whereas the nanobody that is used to competitive elute the target (stripper) has a lower off-rate and/or higher affinity for the target protein, to result in optimal yield and purity. To proof this principle, we performed NANEX experiments with different trapper-stripper pairs targeting the same GFP epitope but vary in affinity and off-rate (k_{off}) in Examples 2-8.

### Example 2: Purification of GFP Protein Using Nb CA12760 as an Immobilized Trapper on NHS-Activated Agarose Beads and Nbs CA12760, CA15818, CA15816, CA15861 as Strippers, Respectively

In this Example, we show that high affinity trappers cannot conveniently be combined with lower affinity strippers.

This example describes the affinity purification of GFP using CA12760 (SEQ ID NO: 1) as the trapper, which is a Nanobody specifically binding GFP with low picomolar affinity. BLI assays identified the affinity (K_{D}) to be 40 pM, kₒₙ 2.56×10⁵ (M⁻¹.s⁻¹) and k_{off} 0.032×10⁻³ (s⁻¹).

To identify the epitope and paratope of GFP⋅CA12760 we solved the structure of this complex by X-ray crystallography (FIG. 3). The crystal comprising GFP (as depicted in SEQ ID NO: 16) and the GFP-Nanobody Nanobody (SEQ ID NO: 1) is in the space group P212121, with the following lattice constants: a=74.497 Å±5%, b=103.450 Å±5%, c=209.774 Å±5%, α=90°, β=90°, γ=90°. This crystal allowed to further delineate the binding site, which is defined herein as being formed by the amino acid residues of the GFP protein (SEQ ID NO: 16), that are in a hydrogen bond with the Nb CA12760, and identified as amino acid Pro89, Glu90, Glu111, Lys113, Phe114, Glu115, and Glyl16 of SEQ ID NO: 16. Based on the structure of GFP⋅CA12760, we identified three key residues (Thr54, Va155, Phe103) to be mutated in the paratope region of the GFP-specific Nb CA12760 in order to decrease the affinity of CA12760 (FIG. 4).
CA15818 Nb has one residue (F103A) mutated to decrease its affinity compared to CA12760. BLI assays identified the affinity (K_{D}) of CA15818 to be 1.5 nM, kₒₙ 3.87×10⁵ (M⁻¹.s⁻¹) and k_{off} 0.29×10⁻³ (s⁻¹) (FIGS. 5A and 5B and Table1).
CA15816 was designed based on the structure of GFP⋅CA12760, two residues (T54A, V55A) are mutated to decrease its affinity compared to CA12760. BLI assays identified the affinity (K_{D}) of CA15816 to be 2.7 nM, kₒₙ 8.35×10⁵ (M⁻¹.s⁻¹) and k_{off} 2.25×10⁻³ (s⁻¹).
CA15861 was designed based on the structure of GFP⋅CA12760, three residues (T54A, V55A, F103) are mutated to decrease its affinity compared to CA12760. BLI assays identified the affinity (K_{D}) of CA15861 to be 111 nM, kₒₙ 28.85×105 (M⁻¹.s⁻¹) and k_{off} 45.8×10⁻³ (s⁻¹).

TABLE 1
GFP-specific Nanobodies with distinct binding kinetics as measured in BLI.

| | | Kon (×10⁵) | Koff | Half- | | Half-time | KD | KD |
|---|---|---|---|---|---|---|---|---|
| Nanobody | Mutations | (M⁻¹ · s⁻¹) | (×10⁻³) (s⁻¹) | time | | ratio | (nM) | ratio |
| CA12760 | Wt | 2.56 | 0.032 | 5 | h | 1 | 0.04 | |
| CA15818 | F103A | 3.87 | 0.29 | 40 | min | 9× | 1.5 | 30× |
| CA15816 | TS4A VSSA | 8.35 | 2.25 | 5.13 | min | 70× | 2.7 | 50× |
| CA15861 | T54A | 28.85 | 45.8 | 15 | s | 1400× | 111 | 2000× |

V55A F103A

Half-time is defined in function of the k_{off} (t_{1/2}=ln2/k_{off}), the half-time ratio is defined here relative to the wt Nb, as well as the K_{D} ratio.

To purify GFP, we covalently immobilized 4 mg of the trapper Nb CA12760 on 500 μl of NHS-Activated agarose beads (Thermo Fisher Scientific) following the supplier's recommendations. 200 μg (7.69 nmol) of purified GFP was loaded on 50 μl CA12760 Nb-coupled agarose beads in a final volume of 1 mL in 100 mM Hepes buffer pH7.5, 150 mM NaCl. To specifically elute GFP protein from this affinity matrix, we used CA12760, CA15818 (SEQ ID NO: 2), CA15816 (SEQ ID NO: 3), CA15861 (SEQ ID NO: 4) as strippers.

The purification of GFP using CA12760 as an immobilized trapper on NHS-Activated agarose beads and CA12760, CA15818, CA15816, CA15861 as a stripper was performed in elution buffer (100 mM Hepes pH7.5, 150 mM NaCl, 53 μM stripper Nb (800 μg/mL)) and monitored by measuring absorbance at 488 nm (indicative of amount of GFP fluorescent protein) at 5 different time points (0, 15, 30, 60, 120 minutes) (FIG. 6). From the absorbance profile of the GFP fluorophore in the elution, we can conclude that using a high affinity trapper Nb with picomolar affinity in combination with the same Nb as the stripper, or using lower affinity Nbs (as compared to the trapper) as strippers, does not cause the fast and quantitative elution of the GFP protein from the beads.

### Example 3. Purification of GFP Using Nb CA15818 as an Immobilized Trapper on NHS-Activated Agarose Beads and Nbs CA12760, CA15818, CA15816, CA15861 as a Stripper

In this Example, a medium affinity trapper was combined with high and low affinity strippers, respectively.

This example describes the affinity purification of GFP protein using CA15818 (see Example 2 and Table 1) as the trapper. To purify GFP, we covalently immobilized 4 mg of the trapper CA15818 on 500 μl of NHS-Activated agarose beads (Thermo Fisher Scientific) following the supplier's recommendations. 200 μg (7.69 nmol) of purified GFP was loaded on 50 μl CA15818 Nb-coupled agarose beads in a final volume of 1 mL in 100 mM Hepes buffer pH7.5, 150 mM NaCl. To specifically elute the GFP from this affinity matrix, we used CA12760, CA15818, CA15816, CA15861 as the stripper (see Table 1).

The purification of GFP protein using Nb CA15818 as an immobilized trapper on NHS-Activated agarose beads and CA12760, CA15818, CA15816, CA15861 as a stripper was performed in elution buffer (100 mM Hepes pH7.5, 150 mM NaCl, 53 μM stripper Nb (800 μg/mL)) and monitored by measuring absorbance at 488 nm at 5 different time points (0, 15, 30, 60, 120 minutes) (FIG. 7). From the absorbance profile of the GFP fluorophore of the elution, we can conclude that using a trapper Nb with nanomolar affinity in combination with a picomolar stripper Nb with an affinity that is about 35-fold higher as the trapper (CA12760 Nb), allows GFP elution to a better extent as compared to the results in Example 2. Moreover, we confirm as in Example 2 that using a trapper Nb with higher affinity than the stripper Nb, does not cause the fast and quantitative elution of the GFP protein from the beads.

### Example 4. Purification of GFP Protein Using Nb CA15816 as an Immobilized Trapper on NHS-Activated Agarose Beads and Nbs CA12760, CA15818, CA15816, CA15861 as a Stripper

In this example, we combined a low affinity trapper with high and low affinity strippers, respectively.

This example describes the affinity purification of GFP using CA15816 Nb as the trapper (See Example 2 and Table 1). To purify GFP, we immobilized covalently 4 mg of the trapper CA15816 Nb on 500 μl of NHS-Activated agarose beads following the supplier's recommendations. 200 μg (7.69 nmol) of purified GFP was loaded on 50 μl CA15816 Nb-coupled agarose beads in a final volume of 1 mL 100 mM Hepes buffer pH7.5, 150 mM NaCl. To specifically elute GFP protein from this affinity matrix, we used CA12760, CA15818, CA15816, and CA15861 Nbs as stripper (see Example 2, Table 1). The purification of GFP using CA15816 Nb as an immobilized trapper on NHS-Activated agarose beads and CA12760, CA15818, CA15816, CA15861 Nbs as a stripper was performed in elution buffer (100 mM Hepes pH7.5, 150 mM NaCl, 53 μM stripper Nb (800 μg/mL)) and was monitored by measuring absorbance at 488 nm at 5 different time points (0, 15, 30, 60, 120 minutes) (FIG. 8). From the absorbance profile of the GFP fluorophore of the elution, we can conclude that using a trapper Nb (CA15816) with nanomolar affinity in combination with a picomolar stripper Nb (CA12760) with an affinity that is about 200-fold higher as the trapper, allows fast and quantitative elution of GFP from the column. Moreover, stripping the beads with slightly higher affinity Nb of only 2-fold (CA15818) or equal affinity (CA15816) allows nearly complete elution of GFP, while using a stripper Nb with a lower affinity of about 100-fold as compared to the trapper, does not allow to recover or elute the GFP protein.

### Example 5. Purification of GFP Protein Using Nb CA15861 as an Immobilized Trapper on NHS-Activated Agarose Beads and Nbs CA12760, CA15818, CA15816, CA15861 as a Stripper

In this Example, we combined a low affinity trapper with higher affinity strippers, respectively.

This example describes the affinity purification of GFP using CA15861 Nb as the trapper (see Example 2 and Table 1). To purify GFP, we covalently immobilized 4 mg of the trapper CA15861 Nb on 500 μl of NHS-Activated agarose beads following the supplier's recommendations. 200 μg (7.69 nmol) of purified GFP protein was loaded on 50 μl CA15861 Nb-coupled agarose beads in a final volume of 1 mL 100 mM Hepes buffer pH7.5, 150 mM NaCl. To specifically elute GFP from this affinity matrix, we used CA12760, CA15818, CA15816, and CA15861 Nbs as the strippers (see Example 2 and Table 1). The purification of GFP using CA15861 as an immobilized trapper on NHS-Activated agarose beads and CA12760, CA15818, CA15816, CA15861 as a stripper was performed in elution buffer (100 mM Hepes pH7.5, 150 mM NaCl, 53 μM stripper Nb (800 μg/mL)) and monitored by measuring absorbance at 488 nm at 5 different time points (0, 15, 30, 60, 120 minutes) (FIG. 9). We can conclude that the affinity of trapper Nb (CA15861) (micromolar range) is too low to trap GFP on the beads.

### Example 6. Purification of GFP Protein by Nanobody Exchange Chromatography, Using Nb CA12760 as an Immobilized Trapper on HiTrap NHS-Activated Sepharose HP Columns and Nbs CA12760, CA15818, CA15816, CA15861 as a Stripper

In this example, we combined a high affinity trapper with lower affinity strippers (analogous to Example 2 but using a trapper that is immobilized on beads that are contained in a prepacked column), for the affinity purification of GFP with CA12760 Nb (see Example 2 and Table 1) coupled to HiTrap NHS-activated Sepharose HP beads using a 1 mL column (1 mL CV), connected to an FPLC (AktaPure-GE) system. To specifically elute the GFP from this affinity matrix, we used CA12760, CA15818, CA15816, CA15861 as the strippers (see Example 2 and Table 1), respectively. 1 mg (66.67 nmol) of CA12760 was immobilized on HiTrap NHS-activated Sepharose HP beads, prepacked in a 1 mL column (GE) following the supplier's recommendations. 2 mg (76.92 nmol) of the GFP was loaded on the CA12760 nb-coupled column through the injection loop. 10 CV of 100 mM Hepes washing buffer pH7.5, 150 mM NaCl was passed over the column to remove unbound material. Elution was performed using 8 CV elution buffer (100 mM Hepes buffer pH7.5, 150 mM NaCl, 66.67 μM stripper Nb (1 mg/mL)) at a flow rate of 0.1 mL/min.

The elution peak was collected in 500 μl fractions and analysed in SDS/PAGE gel (FIGS. 10A-10C). Regeneration of the column was obtained by 8CV of 200 mM Glycine buffer pH2.3.

The purification of GFP protein using CA12760 as an immobilized trapper on HiTrap NHS-activated Sepharose HP column and CA12760, CA15818, CA15816, CA15861 Nbs as a stripper was monitored by measuring the absorbance at 280 nm (Protein absorption) and 488 nm (GFP fluorophore absorption) (FIGS. 10A-10C). From the absorbance profile of the GFP fluorophore in the elution and SDS-PAGE analysis of the eluted fractions, we can conclude that using a trapper Nb with picomolar affinity in combination with the same stripper Nb, or with lower affinity Nbs (as compared to the trapper) as strippers, does not cause the fast and quantitative elution of GFP from the column, while the peak eluted during the regeneration step indicates that most GFP protein was still present on the column and only eluted using low pH (low pH quenches the absorbance of the fluorophore of GFP at 488).

### Example 7. Purification of GFP Protein by Nanobody Exchange Chromatography, Using Nb CA15816 as an Immobilized Trapper on HiTrap NHS-Activated Sepharose HP Columns and Nbs CA12760, CA15818, CA15816, CA15861 as a Stripper

In this example, we combined a low affinity trapper with high and low affinity strippers (analogous as in Example 4 but using a trapper that is immobilized on beads that are contained in a prepacked column), for the affinity purification of GFP with CA15816 Nb (see Example 2, Table 1) coupled to an HiTrap NHS-activated Sepharose HP beads prepacked in a 1 mL column (GE), connected to an FPLC (AktaPure-GE) system. To specifically elute the GFP from this affinity matrix, we used CA12760, CA15818, CA15816, CA15861 as the strippers (see Example 2, Table 1), respectively. 1 mg (66.67 nmol) of CA15816 Nb was immobilized on HiTrap NHS-activated Sepharose HP beads, prepacked in a 1 mL column (1 mL CV) (GE) following the supplier's recommendations. 2 mg (76.92 nmol) of the GFP protein was loaded on the CA15816 Nb-coupled column through the injection loop. 10 CV of washing buffer (100 mM Hepes pH7.5, 150 mM NaCl) was passed over the column to remove unbound material, followed by 8 CV of elution buffer (100 mM Hepes pH7.5, 150 mM NaCl, 66.67 μM stripper (1 mg/mL)- at a flow rate of 0.1 mL/min. The elution peak was collected in 500 μl fractions and analysed in SDS/PAGE gel (FIGS. 11A-11C). Regeneration of the column was obtained by 8 CV of 200 mM Glycine buffer pH2.3.

The purification of GFP protein using CA15816 as an immobilized trapper on HiTrap NHS-activated Sepharose HP column and CA12760, CA15818, CA15816, CA15861 Nbs as a stripper was monitored by measuring the absorbance at 280 nm (Protein absorption) and 488 nm (GFP fluorophore absorption) (FIGS. 11A-11C). From the absorbance profile of the GFP fluorophore in the elution and SDS-PAGE analysis of the eluted fractions, we can conclude that using a trapper Nb (CA15816) with nanomolar affinity in combination with a picomolar stripper Nb (CA12760) with an affinity that is about 200-fold higher as the trapper, allows fast and quantitative elution of GFP from the column, as there is no remaining protein eluted upon regeneration of the column. Moreover, stripping the column with slightly higher affinity Nb of only 2-fold (CA15818) or equal affinity (CA15816) also allows nearly complete elution of GFP, as the 280 nm peak upon regeneration is very low, while using a stripper Nb (CA15861) of about 100-fold lower affinity as compared to the trapper, does not allow to recover or elute the GFP protein.

### Example 8. Purification of GFP Protein by Nanobody Exchange Chromatography, Using Nb CA15861 as an Immobilized Trapper on HiTrap NHS-Activated Sepharose HP Columns and Nbs CA12760, CA15818, CA15816, CA15861 as a Stripper

In this example, we combined a poor trapper with higher affinity strippers (analogous to Example 5 but using a trapper that is immobilized on beads that are contained in a prepacked column), for the affinity purification of GFP CA15861 Nb coupled to HiTrap NHS-activated Sepharose HP beads using a 1 mL column (see Example 2, Table 1), connected to an FPLC (AktaPure-GE) system. To specifically elute GFP from this affinity matrix, we used CA12760, CA15818, CA15816, and CA15861 Nbs as the strippers (Table 1), respectively. 1 mg (66.67 nmol) of CA15861 was immobilized on HiTrap NHS-activated Sepharose HP beads, prepacked in a 1 mL column (1 mL CV; GE) following the supplier's recommendations. 2 mg (76.92 nmol) of GFP was loaded on the CA15861 column through the injection loop. 10 CV of washing buffer (100 mM Hepes pH7.5, 150 mM NaCl) was passed over the column to remove unbound material, followed by 8 CV of elution buffer (100 mM Hepes pH7.5, 150 mM NaCl, 66.67 μM stripper (1 mg/mL) at a flow rate of 0.1 m L/min. The elution peak was collected in 500 μl fractions and analysed in SDS/PAGE gel (FIGS. 12A-12C). Regeneration of the column was obtained by 8 CV of 200 mM Glycine buffer pH2.3. The purification of a GFP using CA15861 Nb as an immobilized trapper on HiTrap NHS-activated Sepharose HP column and CA12760, CA15818, CA15816, CA15861 Nbs as a stripper was monitored by measuring absorbance at 280 nm and 488 nm (FIGS. 12A-12C). From the absorbance profile of the GFP fluorophore in the elution and SDS-PAGE analysis of the eluted fractions, we can conclude that using a trapper Nb (CA15861) with nanomolar to micromolar affinity in combination with higher affinity stripper Nbs allows to recover or elute GFP protein. Considering the very low affinity of the trapper, the yield is somewhat lower, probably because not all the GFP that was loaded onto the column was retained.

### Example 9. Purification of a GFP Protein Spiked in a Bacterial Lysate by NANEX Chromatography Using Nb CA15816 as an Immobilized Trapper on HiTrap NHS-Activated Sepharose HP Columns and Eluted with a CA15621 MegaBody Mb_{CA12760Nb}^{cHopQ} as a Stripper

One major advantage of NANEX is that the stripper can be a functionalized or engineered Nanobody (e.g. a MegaBody, fluorescent labelled for imaging, biotin-coupled for detection) to elute the target in a functionalized complex. To validate these options, we purified GFP from a bacterial lysate by Nanobody exchange chromatography (NANEX) and eluted GFP as a GFP⋅MegaBody complex. Example 9 describes the affinity purification of GFP protein spiked in a bacterial lysate using a HiTrap NHS-activated Sepharose HP column coupled with CA15816 Nb (see Table 1), connected to an FPLC (AktaPure-GE) system. To specifically elute GFP protein from this affinity matrix we used CA15621 Mb as the stripper. CA15621 is a MegaBody, or antigen-binding chimeric protein, as described herein, and with a fusion as disclosed in WO2019/086548A1, in which in particular the Mb_{CA12760Nb}^{cHopQ} is composed of a rigid fusion of the CA12760 GFP-specific Nb with the cHopQ scaffold.

In detail, the 58 kDa MegaBody described here is a chimeric polypeptide concatenated from parts of single-domain immunoglobulin and parts of cHopQ scaffold protein. Here, the immunoglobulin domain used is a GFP-binding Nanobody as depicted in SEQ ID NO: 1. The scaffold protein is an adhesin domain of Helicobacter pylori strain G27 (PDB: 5LP2, SEQ ID NO: 17) called HopQ (Javaheri et al, 2016). The N- and C-terminus of HopQ was connected to allow the creation of a circularly permutated variant of HopQ, called cHopQ, wherein a cleavage of the sequence was made somewhere else in its sequence. To design the Mb_{NbCA12760}^{cHopQ} construct, all parts were connected to each other from the amino (N-) to the carboxy (C-)terminus in the next given order by peptide bonds: β-strand A of the anti-GFP-Nanobody (1-13 of SEQ ID NO: 1), a C-terminal part of HopQ (residues 192-414 of SEQ ID NO: 17), a short peptide linker connecting the C-terminus and the N-terminus of HopQ to produce a circular permutant cHopQ of the scaffold protein, an N-terminal part of HopQ (residues 14-186 of SEQ ID NO: 17), β-strands B to G of the GFP-binding Nanobody (residues 16-126 of SEQ ID NO: 1), 6xHis tag.

For coupling, 1 mg (66.67 nmol) of CA15816 Nb was immobilized on HiTrap NHS-activated Sepharose HP column (1 mL; GE) following the supplier's recommendations. 10 mL of a bacterial lysate (equivalent to the cell pellet of 0.5 L of an E. coli culture grown in LB) was spiked with 2 mg of GFP protein. Lysate was loaded using a syringe on the CA15816 Nb-coupled column, washed twice with 10 CV of washing buffer (100 mM Hepes pH7.5, 150 mM NaCl). The column was then connected on the Akta system (GE), followed with 8 CV of elution buffer (100 mM Hepes pH7.5, 150 mM NaCl, 68.18 μM stripper (4.5 mg/mL)) at a flow rate of 0.1 mL/min. The elution peak was collected in 500 μl fractions and analysed in SDS/PAGE gel (FIG. 13). Regeneration of the column was obtained by 8 CV of 200 mM Glycine buffer pH2.3. The purification of GFP using CA15816 Nb as an immobilized trapper on HiTrap NHS-activated Sepharose HP column and CA15621 Mb_{CA12760Nb}^{cHopQ} as a stripper was monitored by measuring the absorbance at 280 nm (Protein absorption) and 488 nm (GFP fluorophore absorption) (FIG. 13). From the absorbance profile of the GFP fluorophore in the elution and SDS-PAGE analysis of the eluted fractions, we can conclude that using a trapper Nb (CA15816) in combination with higher affinity stripper Nb in its functionalized form as a Mega Body, washing was sufficient for removing most of the impurities, elution fractions 2 and 3 contained the Stripper Mb in complex with the GFP protein as detected in the main peak. So, we can conclude from this that the Mb works similarly well as a stripper compared to its parental Nb CA12760 to elute GFP quantitatively and fast in NANEX.

### Example 10. Purification of GFP Spiked in a Bacterial Lysate by Nanobody Exchange Chromatography, Using Nb CA15816 as an Immobilized Trapper on HiTrap NHS-Activated Sepharose HP Columns and Eluted with a CA15616 MegaBody MbCA12760NbYgik as a Stripper

Example 10 further describes the affinity purification of GFP protein spiked in a bacterial lysate using a HiTrap NHS-activated Sepharose HP column coupled with CA15816 Nb (see Table 1), connected to an FPLC (AktaPure-GE) system, and specifically eluted using CA15816 Mb as the stripper. CA15816 is a MegaBody, or antigen-binding chimeric protein, as described herein, and with a fusion as disclosed in WO2019/086548A1, in which in particular the Mb_{CA12760Nb}^{Ygjk} is composed of a rigid fusion of the CA12760 GFP-specific Nb with the Ygjk scaffold.

In detail, the 100 kDa Megabodies are chimeric polypeptides concatenated from parts of a single-domain immunoglobulin and parts of a scaffold protein linked by short polypeptide linkers. The immunoglobulin used is a GFP-binding Nanobody as depicted in SEQ ID NO: 1. The alternative scaffold protein used was YgjK, a 86 kDA periplasmic protein of E. coli (PDB 3W7S, SEQ ID NO: 32). All parts were connected to each other from the amino to the carboxy terminus in the next given order by peptide bonds: β-strand A of the anti-GFP- Nanobody (residues 1-12 of SEQ ID NO: 1), a peptide linker of one or two amino acids with random composition, the C-terminal part of YgjK (residues 464-760 of SEQ ID NO: 32), a short peptide linker connecting the C-terminus and the N-terminus of YgjK to produce a circular permutant of the scaffold protein, the N-terminal part of YgjK (residues 1-461 of SEQ ID NO: 32), a peptide linker of one or two amino acids with random composition, β-strands B to G of the anti-GFP-Nanobody (residues 17-126 of SEQ ID NO: 1), 6xHis tag.

For coupling, 1 mg (66.67 nmol) of CA15816 was immobilized on HiTrap NHS-activated Sepha rose HP column (1 mL CV; GE) following the supplier's recommendations. 10 mL of a bacterial lysate (equivalent to the cell pellet of 0.5 L of an E. coli culture grown in LB) was spiked with 2 mg of GFP. Lysate was loaded using a syringe on the CA15816 Nb-coupled column, washed twice with 10 CV) of washing buffer (100 mM Hepes pH7.5, 150 mM NaCl). The column was then connected on the Akta system (GE), following 8 CV in elution buffer (100 mM Hepes pH7.5, 150 mM NaCl, 48.54 μM (5 mg/mL) stripper) at a flow rate of The elution peak was collected in 500 μl fractions and analysed in SDS/PAGE gel (FIG. 14). Regeneration of the column was obtained by 8 CV of 200 mM Glycine buffer pH2.3. The purification of a GFP using CA15816 Nb as an immobilized trapper on HiTrap NHS-activated Sepharose HP column and CA15616 Mb_{CA12760Nb}^{Ygjk} as a stripper was monitored by measuring the absorbance at 280 nm (Protein absorption) and 488 nm (GFP fluorophore absorption) (FIG. 14). From the absorbance profile of the GFP fluorophore in the elution and SDS-PAGE analysis of the eluted fractions, we can conclude that using a trapper Nb (CA15816) in combination with higher affinity stripper Nb in its functionalized form as a MegaBody, washing was sufficient for removing most of the impurities, elution fractions 2 and 3 contained the Stripper Mb in complex with the GFP protein as detected in the main peak. So, we can conclude from this that the Mb works similarly well as a stripper compared to its parental Nb CA12760 to elute GFP quantitatively and fast in NANEX.

### Example 11. Purification of GFP Protein by Nanobody Exchange Chromatography Using Nb CA15816 as an Immobilized Trapper on NHS-Activated Agarose Beads to Apply in a Custom-Made 75 μL Micro-Column Using Nb CA12760 as a Stripper

This example describes the affinity purification of a GFP protein on an affinity micro-column, connected to an FPLC (AktaPure-GE) system. We covalently immobilized 4 mg of the trapper CA15816 Nb (see Table 1) on 500 μl of NHS-Activated agarose beads. 75 μl of agarose beads was packed into a custom-made micro-column using commercially available parts that can be connected to common laboratory equipment (FIG. 15). CA12760 Nb was used herein as a stripper (Table 1). 0.1 mg (3.85 nmol) of GFP sample was loaded on the CA15816 Nb-coupled micro-column (75 μl CV) through a 500 μl injection loop. 5 mL (66 CV) of washing buffer (100 mM Hepes pH7.5, 150 mM NaCl) was passed over the column to remove unbound material, followed by 106 CV (8 mL) elution buffer (100 mM Hepes pH7.5, 150 mM NaCl, Volume 500 μl, 13.33 μM stripper Nb (0.2 mg/mL)) at a flow rate of 0.1 mL/min. The elution peak was collected in 500 μl fractions and analysed in SDS/PAGE gel (FIG. 15). Regeneration of the column was obtained by 106 CV of 200 mM Glycine buffer pH2.3. The purification of a GFP using CA15816 Nb as an immobilized trapper on a custom-made micro-column and CA12760 as a stripper was monitored by measuring the absorbance at 280 nm (Protein absorption) and 488 nm (GFP fluorophore absorption) (FIG. 15).

Comparable to the use of larger columns (see Example 7, FIG. 11A-11C), the GFP protein was eluted in fast and quantitatively from this microcolumn.

### Example 12. Purification of a GFP-EPEA Protein by Nanobody Exchange Chromatography Using a CA4375 Synuclein 2-Specific Nb as an Immobilized Trapper on HiTrap NHS-Activated Sepharose HP Columns and Eluted with CA4375 Nb as Monovalent and Bivalent Forms, Respectively as a Stripper

To proof that Nanobody exchange chromatography (NANEX) requires that the immobilized Nanobody (trapper) has a lower affinity and/or higher off-rate, whereas the Nanobody that is used to competitive elute the target has a higher affinity and or lower of rate for the target protein (stripper), to result in optimal yield and purity, we performed NANEX experiments with different trapper-stripper pairs that vary in affinity and off rate. Herein we also describe the EPEA-tag specific Nb as trapper and the same EPEA-specific Nb in monovalent and bivalent version as the stripper. This example describes the affinity purification of GFP-EPEA protein. CA4375 (SEQ ID NO: 7) is a nanobodies selected against human alpha-synuclein (UniProtKB—P37840) that binds a C-terminal linear epitope (EPEA). CA4394 (SEQ ID NO: 8) is a bivalent format of CA4375 Nb selected against human alpha-synuclein (UniProtKB—P37840) that binds a C-terminal linear epitope (EPEA).

BLI assays identified the affinity (K_{D}) of CA4375 to be 60 nM, kₒₙ 6.16×10⁵ (M⁻¹.s⁻¹) and k_{off} 0.324×10⁻³ (s⁻¹). 1 mg (69.89 nmol) of CA4375 monovalent Nb was immobilized on HiTrap NHS-activated Sepharose HP column (1 mL CV; GE) following the supplier's recommendations. 10 mL of a bacterial lysate (0.5 L of culture) was spiked with 2 mg of GFP (76.92 nmol). Lysate was loaded using a syringe on the CA4375 Nb-coupled column, which was then washed twice with 10 CV of buffer (100 mM Hepes pH7.5, 150 mM NaCl). The column was then connected on the Akta system (GE) and followed by 8 CV elution buffer (25 mM HEPES pH 7.5, 150 mM NaCl, concentration stripper CA4375 69.93 μM (1 mg/mL)) and for CA4394 33.57 μM (0.95 mg/mL)) at a flow rate of 0.1 mL/min. The elution peak was collected in 500 μl fractions and analysed in SDS/PAGE gel (FIG. 16 & FIG. 17). Regeneration of the column was obtained by 8 CV of 200 mM Glycine buffer pH2.3.

The purification of GFP-EPEA using CA4375 monovalent Nb as an immobilized trapper on HiTrap NHS-activated Sepharose HP column and CA4394 bivalent as a stripper was monitored by measuring absorbance at 280 nm and 488 nm (FIG. 16 & FIG. 17). From the absorbance profile of the GFP fluorophore in the elution and SDS-PAGE analysis of the eluted fractions, we can conclude that using a monovalent trapper Nb (CA4375) in combination with the same but bivalent Nb (CA4394) as a stripper, washing was sufficient for removing most of the impurities, elution fractions 2 to 5 contained the Stripper Nb and (some) GFP-EPEA protein as detected in the main peak.

In contrast, when the monovalent Nb was used as a stripper, we found that the monovalent Nb only allows to elute little amounts of GFP-EPEA protein. It thus appears that the multivalent stripper has a higher (apparent) affinity (due to avidity effects) and acts as a potent stripper.

### Example 13. Purification of Recombinant Human Synaptojanin Protein by NANEX Using Nb CA13016 as an Immobilized Trapper on HiTrap NHS-Activated Sepharose HP Columns and Using CA13080 Synaptojanin-Specific Nb as a Stripper

Here, we further demonstrate that a lower affinity Synaptojanin-specific Nb can be used as a trapper in combination with unrelated higher affinity Synaptojanin-specific Nb that competes for the same epitope as a stripper to isolate Synaptojanin from cells or cell extracts. This example describes the NANEX chromatography purification of recombinant human Synaptojaninl (amino acid 528-873 of UniProtKB: O43426) in an E. coli cell extract by NANEX. CA13016 Nb (SEQ ID NO: 8) and CA13080 Nb (SEQ ID NO: 9) are Nanobodies selected against the human Synaptojanin1 (528-873).

BLI assays identified the affinity (K_{D}) of CA13016 to be 1.06 μM, kₒₙ 3.8×10⁴ (M⁻¹.s⁻¹) and K_{off} 5×10⁻² (s⁻¹). BLI assays identified the affinity (K_{D}) of CA13080 to be 3.2 nM, kₒₙ 1.8×10⁵ (M⁻¹.s⁻¹) and K_{off} 4×10⁻³ (s⁻¹). Although CA13016 Nb and CA13080 Nb have different CDRs, they are binding to the same epitope on the human Synaptojanin1 and are mutually exclusive. 1 mg (66.57 nmol) of CA13016 Nb was immobilized on HiTrap NHS-activated Sepharose HP column (1 mL CV; GE) following the supplier's recommendations.

Recombinant human Synaptojanin1 (528-873) was expressed using a pET28a expression vector transformed into expression strain BL21(DE3)-T1^{R}, cells were grown in TB media until OD₆₀₀=0.6 and induced with 1 mM IPTG at 20 degrees over-night. Cells were collected by centrifugation and resuspended in 25 mM Hepes (pH7.5), 300 mM NaCl, 10% glycerol, 5 mM MgCl₂, 1 mM DTT (supplemented with DNAse and protein inhibitors) before lysis using a cell-cracker. The crude extract was clarified by centrifugation and supernatant was collected and filtered (0.45 μm filter). 10 mL of lysate (equivalent to the cell pellet of 0.6 L of an E. coli culture grown in LB) was loaded on HiTrap NHS-activated Sepharose HP column coated with CA13016 Nb using a syringe and the flow-through was collected. 10 mL of washing buffer using 25 mM Hepes (pH7.5), 300 mM NaCl, 10% glycerol, 5 mM MgCl2, 1 mM DTT (performed twice) was performed using a syringe and the collected. The affinity column was then connected to an FPLC (AktaPure-GE) system for elution.

Elution conditions (25 mM Hepes (pH7.5), 300 mM NaCl, 10% glycerol, 5 mM MgCl2, 1 mM DTT, Volume 1 mL, concentration stripper (CA13080) 66.97 μM (1 mg/mL), flow rate 0.1 mL/min). After 8 mL (8 CV) the elution buffer is changed for 8 mL (8 CV) of a regeneration buffer (200 mM Glycin pH2.3).

The purification of recombinant human Synaptojanin1 (528-873) using CA13016 Nb as an immobilized trapper on HiTrap NHS-activated Sepharose HP column and CA13080 Nb as a stripper was monitored by measuring absorbance at 280 nm. The elution peak was collected in 500 μl fractions and analysed in SDS/PAGE gel (FIG. 18). From the the absorbance profile of the elution chromatogram and SDS-PAGE analysis of the eluted fractions, we can conclude that using a trapper Nb (CA13016) with micromolar affinity in combination with higher affinity stripper Nb (CA13080), washing was sufficient for removing most of the impurities, elution fractions 2 and 3 contained the Stripper Nb and the human synaptojanin as detected in the main peak (a synaptojanin dimer is also visible on the SDS-PAGE gel). So, we can conclude from this that using a pair of Nbs with different affinities that bind an overlapping epitope is capable of purifying an overexpressed recombinant protein from a bacterial lysate.

### Example 14. Purification of Recombinant Human Coagulation Factor IXa by NANEX Using Nb CA11138 as an Immobilized Trapper on HiTrap NHS-Activated Sepharose HP Columns and Eluted with Nb CA10304 as a Stripper

To further proof the concept we performed a NANEX experiment with a recombinant human coagulation factor IXa specific trapper and eluted factor IXa with a higher affinity stripper. This example describes the Nanobody exchange chromatography of recombinant human coagulation factor IXa (light chain residues 134-191, heavy chain residues 227-461, Uniprot-numbering, UniProtKB—P00740) expressed in E.coli. CA11138 (SEQ ID NO: 11) and CA10304 (SEQ ID NO: 12) are Nanobodies selected against the human coagulation factor IXa.

BLI assays identified the affinity (K_{D}) of CA11138 to be 141 nM, kₒₙ 3.4×10⁴ (M⁻¹.s⁻¹) and k_{off} 4.2×10⁻³ (s⁻¹). BLI assays identified the affinity (K_{D}) of CA10304 to be 46 nM, kₒₙ 8.8×10⁴ (M⁻¹.s⁻¹) and k_{off} 3.5×10⁻³ (s⁻¹).

CA11138 and CA10304 have different CDRs and they are only partially binding to the same epitope on the human coagulation factor IXa and are mutually exclusive. 1 mg (67.25 nmol) of CA11138 was immobilized on HiTrap NHS-activated Sepharose HP column (GE) following the supplier's recommendations. The human coagulation factor IXa was fluorescently labelled using Dylight-647 to follow the purification by measuring absorbance at 650 nm. 0.4 mg of the human coagulation factor IXa fluorescently labelled (Dylight-647) was loaded on the CA11138 column through the injection loop. 10 mL (10 CV) of buffer (20 mM Hepes pH7.5, 150 mM NaCl, 2.5 mM CaCl2) was passed over the column to wash off unbound material. Elution conditions (20 mM Hepes pH7.5, 150 mM NaCl, 2.5 mM CaCl2, Volume 1 mL, concentration CA10304 stripper 468.7 μM (7 mg/mL), flow rate 0.1 m L/min). After 8 mL (8 CV) the elution buffer is changed for 8 ml (8 CV) of a regeneration buffer (200 mM Glycin pH2.3). The purification of the human coagulation factor IXa using CA11138 as an immobilized trapper on HiTrap NHS-activated Sepharose HP column and CA10304 as a stripper was monitored by measuring absorbance at 280 nm and 650 nm. Elution peak was collected in 500 μL fractions and analysed in SDS-PAGE gel (FIG. 19). From the absorbance profile of the elution chromatogram and SDS-PAGE analysis of the eluted fractions, we can conclude that using a trapper Nb (CA11138) in combination with higher affinity stripper Nb (CA10304), washing was sufficient for removing most of the impurities, elution fractions 2, 3 and 4 contained the Stripper and the human coagulation factor IXa as detected in the main peak. So, we can conclude from this that using a competing pair of Nbs with different affinities that bind a partially overlapping epitope is capable of purifying the human coagulation factor IXa.

### Example 15. Purification of the Recombinant Human Coagulation Factor IXa⋅CA10304 Complex by NANEX Using CA10502 as an Immobilized Trapper on HiTrap NHS-Activated Sepharose HP Columns and Eluted with CA10309 as a Stripper

To further proof that a sequential purification using a double NANEX is possible, we performed a NANEX experiment with the eluted recombinant human coagulation factor IXa⋅CA10304 complex from Example 14 for another NANEX purification using a different pair of trapper and stripper that binds to a different epitope as compared to the binders of Example 14.

CA10502 (SEQ ID NO: 13) and CA10309 (SEQ ID NO: 14) are Nanobodies selected against the human coagulation factor IXa. BLI assays identified the affinity (K_{D}) of CA10502 to be 74 nM, kₒₙ 7.0×10⁴ (M⁻¹.s⁻¹) and K_{off} 4.0×10⁻³ (s⁻¹). BLI assays identified the affinity (K_{D}) of CA10309 to be 21 nM, kₒₙ 6.2×10⁴ (M⁻¹.s⁻¹) and K_{off} 7.3×10⁻⁴ (s⁻¹). CA10502 and CA10309 have different CDRs and are partially binding to the same epitope on the human coagulation factor IXa and are mutually exclusive.

1 mg (73.33 nmol) of CA10502 was immobilized on HiTrap NHS-activated Sepharose HP column (GE) following the supplier's recommendations. The human coagulation factor IXa was fluorescently labelled using Dylight-647 to follow the purification by measuring absorbance at 650 nm. 2 mL of the eluted recombinant human coagulation factor IXa(Dylight-647)⋅CA10304 complex from Example 14 was loaded on the CA10502 column through the injection loop. 10 mL (10 CV) of buffer (20 mM Hepes pH7.5, 150 mM NaCl, 2.5 mM CaCl2) was passed over the column to wash off unbound material.

Elution conditions (20 mM Hepes pH7.5, 150 mM NaCl, 2.5 mM CaCl2, Volume 1 mL, concentration CA10309 stripper 52.42 μM (0.75 mg/mL), flow rate 0.1 mL/min). After 8 mL (8 CV) the elution buffer is changed for 8 mL (8 CV) of a regeneration buffer (200 mM Glycine pH2.3). The purification of the human coagulation factor IXa⋅CA10304 complex using CA10502 as an immobilized trapper on HiTrap NHS-activated Sepharose HP column and CA10309 as a stripper was monitored by measuring absorbance at 280 nm and 650 nm. Elution peak was collected in 500 μl fractions and analysed in SDS/PAGE gel (Figure From the absorbance profile of the elution chromatogram and SDS-PAGE analysis of the eluted fractions, we can conclude that using a trapper Nb (CA10502) in combination with higher affinity stripper Nb (CA10309), washing was sufficient for removing most of the impurities and the excess of stripper Nb (CA10304 used in Example 14), elution fractions 2, 3 and 4 contained both strippers (CA10309 and CA10304) and the human coagulation factor IXa as detected in the main peak. The smaller peak at 280 nm and 650 nm after 10 CV of elution represents the remaining amount of sample on the column that was washed off with the regeneration buffer.

So, we can conclude from this that using a pair of Nbs with different affinities that bind a minimal or only partially overlapping epitope is capable of purifying the human coagulation factor IXa in complex with CA10304.

### Example 16. Purification of the Recombinant Human Coagulation Factor IXa by Tandem Nanobody Exchange Chromatography Using CA11138 and CA10502 as Immobilized Trappers on HiTrap NHS-Activated Sepharose HP Columns and Eluted with CA10304 and CA14208 as Strippers

To proof the concept that proteins can be purified by Tandem Nanobody exchange chromatography (tandem-NANEX), we selected two Nanobody pairs that pa irwise compete for two different epitopes on the human coagulation factor IXa according to the scheme described in FIG. 21.

In order to perform the tandem-NANEX we connected a first column (the CA11138 column used in Example 14) to a second column (the CA10502 column used in Example 15).

The first NANEX pair is composed of CA11138 as trapper1 and CA10304 as stripper1, the second NANEX pair is composed of CA10502 as trapper2 and CA14208 (SEQ ID NO: 15) as stripper2. CA14208 is a functionalized Nanobody derived from CA10309 crafted onto the YgjK scaffold to generate a Mega Body. The human coagulation factor IXa was fluorescently labelled using Dylight-647 to follow the purification by measuring absorbance at 650 nm.

0.4 mg of the human coagulation factor IXa fluorescently labelled (Dylight-647) was injected on the columns. 5 mL (2.5 CV) of buffer (20 mM Hepes pH7.5, 150 mM NaCl, 2.5 mM CaCl2) was passed over the column to wash off unbound material. 200 μM of stripper1 (CA10304) was injected from 1.5 mL loop pre-rinsed with buffer, followed by 5 mL (2.5 CV) of buffer. Then 24 μM of Stripper2 (CA14208) was injected on the columns followed by 10 mL (5 CV) of buffer. Next 10 mL of regeneration buffer (200 mM Glycin pH2.3) is applied to remove all proteins from the columns. The tandem-NANEX was monitored by measuring absorbance at 280 nm and 650 nm. Elution peak was collected in 500 μL fractions and analysed in SDS/PAGE gel (FIG. 22). From the SDS-PAGE analysis of the eluted fractions, we can conclude that Factor IXa can be purified using tandem-NANEX by connecting a first column (the CA11138 column used in Example 14) to a second column (the CA10502 column used in Example 15), according to FIG. 21 when using Nb CA10304 and Nb CA14208 stepwise as strippers.

### Example 17. Purification of the Yeast 60S Ribosomal Subunit that Contains the RPP1A-GFP Fusion Protein from a Yeast Extract by NANEX Chromatography, Using Nb CA15816 as an Immobilized Trapper and Nb CA12760 as a Stripper

To proof that Nanobody exchange chromatography (NANEX) works fast and quantitatively if the immobilized Nanobody (trapper) has a lower affinity and/or higher off-rate, whereas the Nanobody that is used to competitive elute the target has a higher affinity and or lower off-rate for the target protein (stripper), to result in optimal yield and purity, we purified the Saccharomyces cerevisiae 60S acidic ribosomal protein P1-alpha (RPP1A, YDL081C, UniProtKB P05318) that contains GFP fused to its carboxy-terminal end from an Saccharomyces cerevisiae extract by NANEX. The yeast clone (reference GFP+35: G8, ThermoFisher Yeast GFP Clone Collection; Hugh et al., 2003) comes from a S. cerevisiae yeast strain collection expressing full-length ORFs containing an Aequorea victoria GFP (S65T) tag (Tsien, 1998) at the C-terminus end. The GFP fusion proteins are integrated into the yeast chromosome through homologous recombination and are expressed using endogenous promoters (Huh et al., 2003).

One mg (66.67 nmol) of CA15816 Nb was immobilized on HiTrap NHS-activated Sepharose HP column (GE) following the supplier's recommendations. 20 mL of a clarified Yeast lysate (equivalent to the cell pellet of 6 L of a culture of yeast clone GFP+35: G8 grown in YPD) was loaded on the CA15816 column using a syringe, washed twice with 10 mL (10 column volumes (CV)) of buffer (100 mM Hepes pH 7.5, 150 mM NaCl). The column was then connected on the Akta pure FPLC system (GE) for elution using the stripper in 8 CV of elution buffer (100 mM Hepes pH 7.5, 150mM NaCl, 66.67 μM stripper Nb CA12760 (1 mg/mL)) at a flow rate of 0.1 mL/min. The elution was collected in 100 μL fractions and the major elution peak was analyzed by SDS/PAGE gel (FIG. 23). Regeneration of the column was obtained by 8 CV of 200 mM Glycine buffer at pH2.3.

The purification of RPP1A-GFP using CA15816 as an immobilized trapper on HiTrap NHS-activated Sepharose HP column and CA12760 as a stripper was monitored by measuring absorbance at 280 nm (protein absorption) and 488 nm (GFP fluorophore absorption) (FIG. 23). From the absorbance profile of the GFP fluorophore in the elution and SDS-PAGE analysis of the eluted fractions, we can conclude that CA15816 can be used as a trapper in combination with CA12760 as a stripper in NANEX to purify RPP1A-GFP from the yeast lysate. Moreover, elution fractions 2 to 11 contained the Stripper Nb in complex with RPP1A-GFP and other components of the yeast ribosome as detected in the main peak. Indeed, we analyzed fraction 6 of the elution peak in negative stain electron microscopy (FIG. 24) and observed large single particles that correspond to the 60s ribosomal subunit of yeast. We can thus conclude from this example that NANEX can be used to purify (an endogenous) protein complex from a cell lysate by using a pair of Nbs with different affinities (CA15816 and CA12760, also used in Examples 1 & 11) that bind an overlapping epitope that is contained in one of the constituting proteins of the multiprotein complex.

### Example 18. Purification of GFP Using Nb CA16695 as an Immobilized Trapper on HiTrap NHS-Activated Sepharose HP and Nb CA16047 as a Stripper

For NANEX, the stripper needs to disrupt the interaction between the trapper and the target to displace the trapper. This can be achieved for a (high affinity) stripper which binds the same or an overlapping epitope on the target as the trapper, but with a higher affinity or lower k_{off}.

To further demonstrate that any stripper-trapper pair that competitively binds an epitope on the target can in principle be used for NANEX, we also identified a stripper-trapper pair (Nb CA16047, Nb CA16695) that competitively binds a different epitope on GFP compared to example 1 (Nb CA12760, Nb CA15816). This example describes the affinity purification of recombinant GFP using CA16695 (SEQ ID NO: 19) as a nanomolar trapper, and CA16047 (SEQ ID NO: 18) as a low nanomolar affinity stripper.

To identify the epitope and the paratope in the GFP⋅CA16047 complex we solved the structure of this complex by X-ray crystallography (FIGS. 25A-25C & 26). The complex comprising GFP (as depicted in SEQ ID NO: 16) and the GFP-Nanobody (SEQ ID NO: 18) crystallized in the space group C121, with the following lattice constants: a=126.91 Å±5%, b=50.35 Å±5%, c=83.13 Å±5%, α=90°, β=130.45°, γ=90°. The crystal structure allowed to further delineate the binding epitope of the stripper-trapper pair, which is defined herein as being formed by all the amino acid residues of the GFP protein (SEQ ID NO: 16), that make a hydrogen bond with the Nb CA16047. This epitope includes amino acids Tyr151, Lys156, Lys158, Lys162, Va1163, Asn164, Lys166, Asp180, Tyr182 of SEQ ID NO: 16. Based on the crystal structure of GFP⋅CA16047, we also identified one key binding residue in the paratope of the GFP-specific Nb (Tyr119) to be mutated aiming to decrease the affinity of CA16047 (FIG. 27).

| | | Kon (×10⁵) | Koff | Half | Half | KD | KD |
|---|---|---|---|---|---|---|---|
| Nb | Mutation | (M⁻¹ · s⁻¹) | (×10⁻³) (s⁻¹) | time | time ratio | (nM) | ratio |
| CA16047 | Wt | 2.34 | 0.98 | 11.79 min | 1 | 6.15 | 1 |
| CA16695 | Y119F | 2.00 | 1.67 | 6.92 min | 1.78× | 8.53 | 1.37× |

Based on the structure of GFP⋅CA16047, Tyr119 of the stripper (CA16047) was mutated to Phe (Tyr119phe) to produce a trapper designated CA16695.
BLI assays identified the affinity (K_{D}) of CA16047 to be 6.15 nM, kₒₙ 2.3×10⁴ (M⁻¹.s⁻¹) and K_{off} 9.8×10⁻⁴ (s⁻¹) (FIG. 27).
BLI assays identified the affinity (K_{D}) of CA16695 to be 8.53 nM, kₒₙ 2.0×10⁵ (M⁻¹.s⁻¹) and K_{off} 1.7×10⁻³ (s⁻¹) (FIG. 27).

Next, we purified GFP by Nanobody exchange chromatography (NANEX) using CA16047 and Nb CA16695 as a stripper-trapper pair and eluted GFP as a GFP Nanobody complex. The affinity purification of GFP protein was performed using Nb CA16695 coupled to a HiTrap NHS-activated Sepharose HP column, connected to an FPLC (AktaPure-GE) system. To specifically elute GFP protein from this affinity matrix we used CA16047 Nb as the stripper.

For coupling, 1 mg (66.13 nmol) of CA16695 Nb was immobilized on a commercial HiTrap NHS-activated Sepharose HP column (1 mL; GE) following the supplier's recommendations. 2 mg of GFP was loaded using a syringe on the CA16695 Nb-coupled column. The column was washed twice with 10 CVs of washing buffer (100 mM Hepes pH7.5, 150 mM NaCl). The column was then connected to an Akta system (GE). GFP was eluted with 8 CV of elution buffer (100 mM Hepes pH7.5, 150 mM NaCl) containing 66.06 μM of CA16047 stripper Nb (1 mg/mL) at a flow rate of 0.1 mL/min. The purification of a GFP using CA16695 Nb as an immobilized trapper on HiTrap NHS-activated Sepharose HP column and CA16047 as a stripper was monitored by measuring the absorbance at 280 nm (Protein absorption) and 488 nm (GFP fluorophore absorption) (FIGS. 28A and 28B). The elution peak was collected in 500 μl fractions and analysed on an SDS/PAGE gel (FIGS. 28A and 28B). The column was regenerated with 8 CVs of 200 mM Glycine buffer at pH2.3. From the absorbance profile of the GFP fluorophore in the elution and SDS-PAGE analysis of the eluted fractions, we conclude that using a trapper Nb (CA16695) in combination with higher affinity stripper Nb (CA16047) allowed the purification of the GFP protein. Elution fractions 3 to 9 contained the Stripper Nb in complex with the GFP protein as detected in the main peak, showing that this Nb pair (CA16695 as trapper and CA16047 as stripper) enables the quantitative and fast purification of GFP and GFP-tagged proteins by NANEX.

### Example 19. Purification of GST Protein Using Nb CA16240 as an Immobilized Trapper on HiTrap NHS-Activated Sepharose HP Columns and Nbs CA16239 as a Stripper

Glutathione S-transferase (GST) is often used as a GST-tag to separate and purify proteins that contain the GST-fusion protein. This example describes the affinity purification of GST using CA16240 (SEQ ID NO: 21) as the trapper, which is a Nanobody specifically binding GST with low nanomolar affinity and CA16239 (SEQ ID NO: 20) as a low nanomolar affinity stripper.

To identify the epitope and paratope in the GSTCA16239 interaction we solved the structure of this complex by X-ray crystallography (FIGS. 29A-29C & 30). The crystal comprising GST (as depicted in SEQ ID NO: 22) and the GST-Nanobody (SEQ ID NO: 20) is in the space group C121, with the following lattice constants: a=69.866 Å±5%, b=72.451 Å±5%, c=79.710 Å±5%, α=90°, β=93.61°, γ=90°. This crystal structure allowed to further delineate the epitope, which is defined herein as being formed by the amino acid residues of the GST protein (SEQ ID NO: 22), that make a hydrogen bond with the Nb CA16239. This epitope includes amino acids Asp160, Tyr164, Pro167, Leu170, Asp171, Lys181, Glu184, His215 of SEQ ID NO: 22. Based on the crystal structure of GSTCA16239, we also identified one key residues (Tyr109) in the paratope region of the GST-specific Nb to be mutated aiming to decrease the affinity of CA16239 (FIG. 30).

| | | Kon (×10⁵) | Koff | Half | Half | KD | KD |
|---|---|---|---|---|---|---|---|
| Nb | Mutation | (M⁻¹ · s⁻¹) | (×10⁻³) (s⁻¹) | time | time ratio | (nM) | ratio |
| CA16239 | Wt | 7.20 | 3.09 | 224.27 s | 1 | 4.86 | 1 |
| CA16240 | Y109A | 5.12 | 251 | 2.67 s | 84× | 659 | 135.6× |

Based on the structure GST⋅CA16239, one residue (Y109) of the stripper (CA16239) was mutated to Alanine (Y109A) to produce a trapper designated CA16240.
BLI assays identified the affinity (K_{D}) of CA16239 to be 4.9 nM, kₒₙ 7.2×10⁵ (M⁻¹.s⁻¹) and K_{off} 3.1×10⁻³ (s⁻¹) (FIG. 31).
BLI assays identified the affinity (K_{D}) of CA16240 to be 659 nM, kₒₙ 5.1×10⁵ (M⁻¹.s⁻¹) and K_{off} 2.5×10⁻¹ (s⁻¹) (FIG. 31).

Next, we purified GST by Nanobody exchange chromatography (NANEX) using CA16239 and CA16240 as a stripper-trapper pair and eluted GST as a GSTNanobody complex. The affinity purification of GST protein was performed using Nb CA16240 coupled to a HiTrap NHS-activated Sepharose HP column c, connected to an FPLC (AktaPure-GE) system. To specifically elute GFP protein from this affinity matrix we used CA16239 Nb as the stripper.

For coupling, 5 mg (326.5 nmol) of CA16240 Nb was immobilized on a commercial HiTrap NHS-activated Sepharose HP column (1 mL; GE) following the supplier's recommendations. 2 mg of GST protein was loaded using a syringe on the CA16240 Nb-coupled column, washed twice with 10 CV of washing buffer (100 mM Hepes pH7.5, 150 mM NaCl). The column was then connected on the Akta system (GE), followed with 8 CVs of elution buffer (100 mM Hepes pH7.5, 150 mM NaCl), containing 129.82 μM of CA16239 stripper Nb (2 mg/mL) at a flow rate of 0.1 mL/min. The purification of a GST using CA16240 Nb as an immobilized trapper on HiTrap NHS-activated Sepharose HP column and CA16239 as a stripper was monitored by measuring the absorbance at 280 nm (Protein absorption) (FIGS. 32A and 32B). The elution peak was collected in 500 μl fractions and analysed in SDS/PAGE gel (FIGS. 32A and 32B). Regeneration of the column was obtained by 8 CV of 200 mM Glycine buffer pH2.3. From the absorbance profile and the SDS-PAGE analysis of the eluted fractions, we conclude that using a trapper Nb (CA16240) in combination with higher affinity stripper Nb (CA16239), with a much lower k_{off}, but nearly equal kₒₙ allowed the purification of the GST protein. Elution fractions 3 to 7 contained the stripper Nb in complex with the GST protein as detected in the main peak, showing that this Nb pair (CA16240 as trapper and CA16239 as stripper) enables the quantitative and fast purification of GST or GST-tagged proteins by NANEX.

### Example 20. Purification of SMT3 Protein Using Nb CA16687 as an Immobilized Trapper on HiTrap NHS-Activated Sepharose HP Columns and Nbs CA15839 as a Stripper

Ubiquitin-like protein SMT3 (is the yeast SUMO protein, smt3) is often used as a smt3-tag to separate and purify proteins that contain the smt-3-fusion protein.

This example describes the affinity purification of SMT3 using CA16687 (SEQ ID NO: 24) as the trapper, which is a Nanobody specifically binding SMT3 with low nanomolar affinity and CA15839 (SEQ ID NO: 25) as a low nanomolar affinity stripper. To identify the epitope and paratope in the SMT3CA15839 interaction we solved the structure of this complex by X-ray crystallography (FIGS. 33A-33C & 34). The crystal comprising SMT3 (as depicted in SEQ ID NO: 25) and the SMT3-Nanobody (SEQ ID NO: 23) is in the space group P1211, with the following lattice constants: a=45.71 Å±5%, b=90.66 Å±5%, c=57.75 Å±5%, α=90°, β=112.36°, γ=90°. This crystal structure allowed to further delineate the epitope, which is defined herein as being formed by the amino acid residues of the SMT3 protein, that make a hydrogen bond with the Nb CA15839. This epitope includes amino acids His21, Asn23, Phe34, Lys36, Lys38, Arg45, Asn84 of SEQ ID NO: 25.

Based on the structure of SMT3CA15839, we also identified one key residue (Asp50) in the paratope of the SMT3-specific Nb to be mutated aiming to decrease the affinity of CA15839 (FIG. 34).

| | | Kon (×10⁵) | Koff | Half | Half | KD | KD |
|---|---|---|---|---|---|---|---|
| Nb | Mutation | (M⁻¹ · s⁻¹) | (×10⁻³) (s⁻¹) | time | time ratio | (nM) | ratio |
| CA15839 | Wt | 3.18 | 2.12 | 5.45 min | 1 | 6.67 | 1 |
| CA16687 | D50A | 3.87 | 7.97 | 1.45 min | 3.75× | 29 | 4.34× |

Based on the structure of SMT3CA15839, one residue (Asp50) of the stripper (CA15839) was mutated to Alanine (D50A) to produce a trapper designated CA16687.
BLI assays identified the affinity (KD) of CA15839 to be 6.7 nM, kon 3.2×105 (M⁻¹.s⁻¹) and K_{off} 2.1×10−3 (s⁻¹) (FIG. 35).
BLI assays identified the affinity (KD) of CA16687 to be 29 nM, kon 3.9×105 (M⁻¹.s⁻¹) and K_{off} 8.0×10−3 (s⁻¹) (FIG. 35).

Next, purified SMT3 by Nanobody exchange chromatography (NANEX) using CA15839 and CA16687 as a stripper-trapper pair and eluted SMT3 as a SMT3Nanobody complex. The affinity purification of SMT3 protein was performed using Nb CA16687 coupled to a HiTrap NHS-activated Sepharose HP column coupled with, connected to an FPLC (Akta Pure-GE) system. To specifically elute SMT3 protein from this affinity matrix we used CA15839 Nb as the stripper.

For coupling, 1 mg (69.58 nmol) of CA16687 Nb was immobilized on a HiTrap NHS-activated Sepharose HP column (1 mL; GE) following the supplier's recommendations. 2 mg of SMT3 protein was loaded using a syringe on the CA16687 Nb-coupled column, washed twice with 10 CV of washing buffer (100 mM Hepes pH7.5, 150 mM NaCl). The column was then connected on the Akta system (GE), followed with 8 CVs of elution buffer (100 mM Hepes pH7.5, 150 mM NaCl), containing 138.73 μM of CA15839 stripper Nb (2 mg/mL) at a flow rate of 0.1 mL/min. The purification of a SMT3 using CA16687 Nb as an immobilized trapper on HiTrap NHS-activated Sepharose HP column and CA15839 as a stripper was monitored by measuring the absorbance at 280 nm (Protein absorption) (FIGS. 36A and 36B). The elution peak was collected in 500 μl fractions and analysed in SDS/PAGE gel (FIGS. 36A and 36B). Regeneration of the column was obtained by 8 CVs of 200 mM Glycine buffer pH2.3. From the absorbance profile and the SDS-PAGE analysis of the eluted fractions, we can conclude that using a trapper Nb (CA16687) in combination with higher affinity stripper Nb (CA15839)), with a lower k_{off}, but nearly equal kₒₙ allowed the purification of the SMT3 protein. Elution fractions 3 to 8 contained the stripper Nb in complex with the SMT3 protein as detected in the main peak, showing that this Nb pair (CA16687 as trapper and CA15839 as stripper) enables the quantitative and fast purification of SMT3 and SMT3-tagged proteins by NANEX. Therefore, the SMT3-NANEX pair can be used to specifically purify post-translationally modified proteins from yeast.

### Example 21. Purification of an mCherry-Fusion Protein Using Nb CA16964 as an Immobilized Trapper on HiTrap NHS-Activated Sepharose HP Columns and Nbs CA17302 as a Stripper

mCherry is a member of the mFruits family of monomeric red fluorescent proteins derived from DsRed of Discosoma sea anemones. Similar to GFP, mCherry is often used to tag proteins in the cell, so they can be studied using fluorescence spectroscopy and fluorescence microscopy. This example describes the affinity purification of an mCherry-fusion protein using Nb CA16964 (SEQ ID NO: 26) as the trapper, which is a Nanobody specifically binding mCherry with low nanomolar affinity and Nb CA17302 (SEQ ID NO: 27) as a low nanomolar affinity stripper.

Nbs CA16964 and CA17302 are two unrelated Nanobodies from a different sequence family that were generated by immunization of a llama with mCherry and selected by phage display against this antigen following standard procedures (Pardon, 2014). Epitope mapping using BLI indicated that CA17302 and CA16964 compete for an overlapping epitope on mCherry and bind this fluorescent protein in a mutually exclusive manner (FIGS. 37A and 37B). Both Nbs were further characterized in BLI (FIG. 38), with the following values:

| Nb | Kon (×10⁵) (M⁻¹ · s⁻¹) | Koff (×10⁻³) (s⁻¹) | Half time | Half time ratio | KD (nM) | KD ratio |
|---|---|---|---|---|---|---|
| CA17302 | 6.19 | 1.02 | 11.32 min | 1 | 1.69 | 1 |
| CA16964 | 6.94 | 4.36 | 2.65 min | 4.27× | 6.53 | 3.86× |

BLI assays identified the affinity (K_{D}) of CA17302 to be 1.7 nM, kₒₙ 6.2×10⁵ (M⁻¹.s⁻¹) and K_{off} 1×10⁻³ (s⁻¹). (FIG. 38).
BLI assays identified the affinity (K_{D}) of CA16964 to be 6.53 nM, kₒₙ 6.94×10⁵ (M⁻¹.s⁻¹) and K_{off} 4.4×10⁻³ (s⁻¹). (FIG. 38).

Next, we purified FmIH_lectin_mCherry_his (SEQ ID NO: 29), a fusion protein containing mCherry, the FmIH lectin from an uropathogenic E. coli strain and a short His-Tag by Nanobody exchange chromatography (NANEX) using Nbs CA17302 and CA16964 as a stripper-trapper pair to elute an FmIH_lectin_mCherry_his⋅Nanobody complex. The affinity purification was performed using Nb CA16964 coupled to HiTrap NHS-activated Sepharose HP, connected to an FPLC (Akta Pure-GE) system. To specifically elute the FmIH_lectin_mCherry_his fusion protein from this affinity matrix we used CA17302 Nb as the stripper.

For coupling, 1 mg (68 nmol) of CA16964 Nb was immobilized on HiTrap NHS-activated Sepharose HP column (1 mL; GE) following the supplier's recommendations. 50 mL of lysate (from a 2 L bacterial culture of overexpressed recombinant FmIH_lectin_mCherry_his) was loaded using a syringe on the CA16964 Nb-coupled column, washed twice with 10 CVs of washing buffer (100 mM Hepes pH7.5, 150 mM NaCl). The column was then connected on the Akta system (GE), followed with 8 CVs of elution buffer (100 mM Hepes pH7.5, 150 mM NaCl), containing 65.5 μM of CA17302 stripper Nb (1 mg/mL) at a flow rate of 0.1 mL/min. The purification of a FmIH_lectin_mCherry_his using CA16964 Nb as an immobilized trapper and CA17302 as a stripper was monitored by measuring the absorbance at 280 nm (Protein absorption) (FIGS. 39A and 39B). The elution peak was collected in 500 μl fractions and analysed in SDS/PAGE gel (FIGS. 39A and 39B). Regeneration of the column was obtained by 8 CVs of 200 mM Glycine buffer pH2.3. From the elution chromatogram and the SDS-PAGE analysis of the eluted fractions, we can conclude that using a trapper Nb (CA16964) in combination with higher affinity stripper Nb (CA17302)), with a lower k_{off}, but nearly equal kₒₙ allowed the purification of the FmIH_lectin_mCherry_his protein. Elution fractions 4 to 8 contained the stripper Nb in complex with the FmIH_lectin_mCherry_his fusion protein as detected in the main peak, showing that this Nb pair (CA16964 as trapper and CA17302 as stripper) enables the quantitative and fast purification of mCherry fusion proteins by NANEX.

### Example 22. Purification of an mCherry-Fusion Protein Using Nb CA17341 as an Immobilized Trapper on HiTrap NHS-Activated Sepharose HP Columns and Nb CA17302 as a Stripper

This example describes the affinity purification of the FmIH_lectin_mCherry_his fusion protein using Nb CA17341 (SEQ ID NO: 28) as the trapper, which is a Nanobody specifically binding mCherry with low nanomolar affinity and Nb CA17302 (SEQ ID NO: 27) as a low nanomolar affinity stripper. Nb CA17341 was obtained starting from Nb CA17302 by performing an alanine scan on the CDR3 of Nb CA17302. Mutating Ile101 to an alanine allowed us to convert a stripper to a trapper without any a priori structural information on the Nb⋅antigen interactions. CA17341 was further characterized in BLI (FIG. 40) with the following values:

| | | Kon (×10⁵) | Koff | Half | Half | KD | KD |
|---|---|---|---|---|---|---|---|
| Nb | Mutation | (M⁻¹ · s⁻¹) | (×10⁻³) (s⁻¹) | time | time ratio | (nM) | ratio |
| CA17902 | Wt | 6.19 | 1.02 | 11.32 min | 1 | 1.69 | 1 |
| CA17341 | I101A | 6.01 | 2.31 | 5 min | 2.26× | 4.05 | 2.4× |

BLI assays identified the affinity (K_{D}) of CA17302 to be 1.7 nM, kₒₙ 6.2×10⁵ (M⁻¹.s⁻¹) and K_{off} 1×10⁻³ (s⁻¹). (FIG. 40).
BLI assays identified the affinity (K_{D}) of CA17341 to be 4.1 nM, kₒₙ 6×10⁵ (M⁻¹.s⁻¹) and K_{off} 2.3×10⁻³ (s⁻¹). (FIG. 40).

Next, we purified the FmIH_lectin_mCherry_his fusion protein by Nanobody exchange chromatography (NANEX) using Nb CA17341 and Nb CA17302 as the stripper-trapper pair and eluted the target as a FmIH_lectin_mCherry_his⋅Nanobody complex. The affinity purification was performed with Nb CA17341 immobilised on a HiTrap NHS-activated Sepharose HP column, that was connected to an FPLC (AktaPure-GE) system. To specifically elute the FmIH_lectin_mCherry_his fusion protein from this affinity matrix we used CA17302 Nb as the stripper.

For coupling, 1 mg (65.69 nmol) of CA17341 Nb was immobilized on HiTrap NHS-activated Sepharose HP column (1 mL; GE) following the supplier's recommendations. 50 mL of a cell lysate (harvested from a 2 L culture of E. coli overexpressing the FmIH_lectin_mCherry_his fusion protein) was loaded on the CA16964 Nb-coupled column. The column was washed twice with 10 CVs of washing buffer (100 mM Hepes pH7.5, 150 mM NaCl) and connected on the Akta system (GE). The fusion protein was next eluted from this column with 8 CVs of elution buffer (100 mM Hepes pH7.5, 150 mM NaCl), containg 65.5 μM of CA17302 stripper Nb (1 mg/mL) at a flow rate of 0.1 mL/min. The purification of a FmIH_lectin_mCherry_his using CA17341 Nb as an immobilized trapper on HiTrap NHS-activated Sepharose HP column and CA17302 as a stripper was monitored by measuring the absorbance at 280 nm (Protein absorption) and at 585 nm (mCherry absorption) (FIGS. 41A and 41B). The elution peak was collected in 500 μl fractions and analysed in SDS/PAGE gel (FIGS. 41A and 41B). Regeneration of the column was obtained by 8 CVs of 200 mM Glycine buffer pH2.3. From the SDS-PAGE analysis of the eluted fractions, we can conclude that using a trapper Nb (CA17341) in combination with higher affinity stripper Nb (CA17302) allowed the purification of the FmIH_lectin_mCherry_his protein. Elution fractions 4 to 8 contained the stripper Nb in complex with the FmIH_lectin_mCherry_his fusion protein as detected in the main peak, indicating that this Nb pair (CA17341 as trapper and CA17302 as stripper) enables the fast and quantitative purification of mCherry fusions protein by NANEX. The example also shows that a simple alanine scan of the CDR3 is a fast and easy method to convert a stripper into a trapper without the need of structural information.

### Example 23: Purification of Native Human Coagulation Factor IX Using Nb CA11143 as an Immobilized Trapper on a HiTrap NHS-Activated Sepharose HP, and MegaBody CA16383 (an Engineered Antigen Binding Protein Derived from Nb CA14208) as the Stripper

The concept of Nanobody exchange chromatography (NANEX) is not restricted to the use of Nanobodies as strippers and or trappers, respectively. In fact, any pair of proteins that competes for the binding to the same epitope of the target can be used to purify this target following the principle of NANEX: antibodies, megabodies, darpins, synthetic binding proteins, . . . In this Example, we show that a Nanobody can be used in combination with a MegaBody to purify human coagulation factor IX following the principle of NANEX. A Mega Body is an engineered antigen binding protein that is obtained by grafting Nanobodies onto selected protein scaffolds to increase their molecular weight while retaining the full antigen binding specificity and affinity, as previously described. This example is similar to example 16 except that we used a MegaBody (CA16383) derived from Nb CA14208 as the stripper. In this way, we used NANEX to purify a native protein from its natural source (human blood) to purify native human coagulation factor IX from human plasma in complex with a MegaBody, ready for structural characterization by cryo-EM.

More specific, this example describes the affinity purification of human coagulation factor IX from human recovered plasma treated with ACD anti-coagulant using immobilized Nanobody CA11143 (SEQ ID NO: 31) as the trapper, and Mega Body CA16383 (SEQ ID NO: 30) as a stripper.

For coupling, 3.7 mg (250.97 nmol) of CA11143 Nb was immobilized on HiTrap NHS-activated Sepharose HP column (1 mL; GE) following the supplier's recommendations. 30 mL of human recovered plasma treated with ACD anticoagulant (Tebu-Bio, SER-PLE200ML-ACD) was loaded on the CA11143 Nb-coupled column by recirculation for 120 minutes using a peristaltic pump. Next, the column was washed with 15 CVs of washing buffer (20 mM Hepes pH 8.0, 150 mM NaCl, 5 mM CaCl₂) The column was then connected onto an Akta system (GE), and factor IX was eluted with 1 mL of buffer (20 mM Hepes pH 8.0, 150 mM NaCl, 5 mM CaCl₂) containing 9.92 μM of Mega Body CA16383 that was used as the stripper at a flow rate of 0.05 mL/min. The purification process was monitored by measuring the absorbance at 280 nm (Protein absorption) (FIGS. 42A-42C). The elution peak was collected in 750 μl fractions and the fractions were analyzed in SDS/PAGE gel and by western blot including a commercial human coagulation factor IX as a positive control (FIGS. 42A-42C). Regeneration of the column was obtained by 5 CV of 200 mM Glycine buffer pH 2.3. From the SDS-PAGE analysis of the eluted fractions, we can conclude that using a trapper Nb (CA11143) in combination with a functionalized stripper Nb (CA16383) allowed the purification of the human coagulation factor IX protein from blood plasma. Fractions 4 and corresponding to the main elution peak contained the purified human coagulation factor IX protein in complex with the MegaBody, indicating that megabodies can be combined with Nanobodies as stripper-trapper pairs to purify native proteins from natural complex sources.

### Example 24: Purification of Native Human Coagulation Factor IX Using MegaBody CA16388, as an Immobilized Trapper on a HiTrap NHS-Activated Sepharose HP, and Nb CA16383, a Functionalized Nanobody (MegaBody Mb_{CA10309}^{YgjK}), as a Stripper

As shown under example 23, Nanobodies can be used as a trapper in combination with a MegaBody to strip targets from the functionalized resin. In example 24 we used a MegaBody as the trapper in combination with another MegaBody as the stripper to purify the human coagulation factor IX from human blood serum. To proof this principle, we performed NANEX experiments to purify native human coagulation factor IX from human plasma treated with ACD anti-coagulant usingMegaBody CA16388 as the trapper end MegaBody CA16383 as the stripper to purify human coagulation factor IX in complex with MegaBody CA16383 by NANEX.

For coupling, 1.1 mg (10.87 nmol) of MegaBody CA16388 was immobilized on HiTrap NHS-activated Sepharose HP column (1 mL; GE) following the supplier's recommendations. 30 mL of human recovered plasma treated with ACD anticoagulant (Tebu-Bio, SER-PLE200ML-ACD) was loaded on the CA16388 Nb-coupled column by recirculation for 60 minutes using a peristaltic pump the column was washed with 15 CVs of washing buffer (20 mM Hepes pH 8.0, 150 mM NaCl, 5 mM CaCl₂). The washed column was then connected on the Akta system (GE), and factor IX was eluted with 1 mL of buffer (20 mM Hepes pH 8.0, 150 mM NaCl, 5 mM CaCl₂) containing 9.92 μM of Mega Body CA16383 stripper as the stripper at a flow rate of 0.05 mL/min. The purification process was monitored by measuring the absorbance at 280 nm (Protein absorption) (FIGS. 43A-43C). The elution peak was collected in 750 μl fractions and analysed in SDS/PAGE gel and western blot including commercial human coagulation factor IX as a control (FIGS. 43A-43C). Regeneration of the column was obtained by 5 CVs of 200 mM Glycine buffer pH 2.3. From the SDS-PAGE and Western blot analysis of the eluted fractions, we can conclude that MegaBody CA16388 and MegaBody CA16383 as a trapper-stripper pair for the purification of the human coagulation factor IX protein from blood plasma by NANEX. Elution fractions 4 and 5 contained the Stripper Nb in complex with the human coagulation factor IX protein as detected in the main peak.

### Example 25. Purification of the Human GFP-Tagged Glucocorticoid Receptor (GFP-GR) in Complex with its Native Molecular Chaperones from a HEK293T Cell Lysate Using Nb CA15816 as an Immobilized Trapper on a HiTrap NHS-Activated Sepharose HP, and Nb CA12670 as a Stripper

As the NANEX technology does not require high salt concentrations or extreme pH conditions for elution and can be performed entirely under native conditions (pH, buffer composition, temperature, . . . ) this method is also applicable for the purification of (transient) protein-protein complexes from native sources. To proof this principle, we tagged the human glucocorticoid receptor (GR) with GFP, expressed this fusion protein in a human cell line and purified the receptor in complex with its molecular chaperones Hsp70 and Hsp90 (heat shock proteins). It is known from literature that, apo-GR is predominantly cytoplasmic and associated with heat shock proteins and immunophilins in the so-called foldosome in its resting state (Pratt et al., 1997). Accordingly, we purified the eGFP-tagged recombinant human glucocorticoid receptor (SEQ ID NO: 34) in complex with Hsp70 and Hsp90 from human HEK293T cells using CA15816 (SEQ ID NO: 3) as an immobilized trapper (medium-affinity trapper for GFP) on a HiTrap NHS-activated Sepharose HP column, and Nb CA12670 (SEQ ID: 1) as a high-affinity stripper for the GFP-tagged GR receptor in association with its molecular chaperones.

For coupling, 1 mg (66.955 nmol) of CA15816 Nb was immobilized on HiTrap NHS-activated Sepharose HP column (1 mL; GE) following the supplier's recommendations. Recombinant human eGFP-6His-TEV-GR was expressed using a pcDNA3.1+N-eGFP vector transfected into human HEK239T cells grown in 150×21 mm dishes (Nunclon™ Delta) using X-tremeGENE™ 9 DNA Transfection Reagent (XTG9-RO Roche) for transient expression. After transfection, HEK239T cells were grown for 48 h at 37° C., 5% CO₂. Cells from 3 plates were collected by pipetting and centrifugation, washed with PBS and resuspended in 10 mL lysis buffer containing 10 mM Na-Phosphate pH8, 5 mM DTT, 0.1 mM EDTA, 10 mM Na₂MoO₄, 10% Glycerol (supplemented with protease inhibitors) before lysis using a Dounce homogenizer. The lysate was clarified by centrifugation and the supernatant was collected, filtered (0.45 μm filter) and loaded onto a HiTrap NHS-activated Sepharose HP column coated with CA15816 Nb using a syringe. 10 CVs of washing buffer (50 mM Hepes pH7.5, 150 mM NaCl) were applied on the column using a syringe. The affinity column was then connected to an FPLC (AktaPure-GE) system and the foldosome was eluted with 1 mL containing 66.7 μM CA15816 Nb stripper (1 mg/mL) in 50 mM Hepes pH7.5, 150 mM NaCl at a flowrate flow rate 0.1 mL/min. Regeneration of the column was obtained using 5 CVs of 200 mM Glycine buffer pH 2.3. The purification process was monitored by measuring the absorbance at 280 nm (Protein absorption) and 488 nm (eGFP) (FIGS. 44A-44C). The elution peak was collected in 500 μL fractions and analyzed in SDS-PAGE gel and western blot using a commercial anti-human glucocorticoid receptor antibody (anti-GR G-5, Santa Cruz) (FIGS. 44A-44C). The three major bands observed on SDS-PAGE analysis of the eluted fractions were cut and analysed by Mass Spectrometry. The major bands were identified as human glucocorticoid receptor, HSP90 and HSP70 proteins, respectively (FIGS. 44A-44C). Hsp70 and Hsp90 are molecular chaperones, known to form a complex with nuclear receptors such as the human glucocorticoid receptor in the cytoplasm (foldosome), showing that NANEX allows fast and easy purification of a native protein-protein complex containing amongst others eGFP-tagged GR, Hsp70, Hsp90 and the stripper from transfected HEK293T cells.

### Example 26. Purification of the Recombinant Human GFP-Tagged Androgen Receptor (GFP-ARb) in Complex with Molecular Chaperones from HEK293T Cells Lysate Using Nb CA15816 as an Immobilized Trapper on a HiTrap NHS-Activated Sepharose HP, and Nb CA12670 as a Stripper

As demonstrated in example 25, NANEX can also be used to purify (transient) protein complexes. To further substantiate this principle, we also purified the human androgen receptor from HEK293T cells in complex with its molecular chaperones Hsp70 and Hsp90 and immunophilins in the so-called foldosome (Pratt et al., 1997).

This example describes the NANEX purification of the eGFP-tagged recombinant human androgen receptor (SEQ ID NO: 35) in complex with Hsp70 and Hsp90 from human HEK293T cells lysate using CA15816 (SEQ ID NO: 3) as an immobilized trapper (medium-affinity trapper for GFP), and Nb CA12670 (SEQ ID: 1) as a stripper (high-affinity stripper for GFP).

For coupling, 1 mg (66.955 nmol) of CA15816 Nb was immobilized on HiTrap NHS-activated Sepharose HP column (1 mL; GE) following the supplier's recommendations.

Recombinant human eGFP-6His-TEV-AR was expressed using a pcDNA3.1+N-eGFP vector transfected into human HEK239T cells. Cells were transfected in 150×21 mm dishes (Nunclon™ Delta) using X-tremeGENE™ 9 DNA Transfection Reagent (XTG9-RO Roche) with a ratio 3:1 μL/μg DNA. After transfection, HEK239T cells were grown for 48 h at 37° C. and 5% CO₂. Cells from 3 plates were collected by pipetting and centrifugation, washed with PBS and resuspended in 10 mL lysis buffer 10 mM Hepes 7.5, 2.5 mM DTT, 1 mM EDTA, 20 mM Na₂MoO₄, 10% Glycerol (supplemented with protease inhibitors) before lysis using a Dounce homogenizer. The lysate was clarified by centrifugation and the supernatant was collected, filtered (0.45 μm filter) and loaded on a HiTrap NHS-activated Sepharose HP column coated with CA15816 Nb using a syringe. Next, the column was washed with 10 CVs of washing buffer (10 mM Hepes 7.5, 2.5 mM DTT, 1 mM EDTA, 20 mM Na₂MoO₄, 10% Glycerol) using a syringe. The affinity column was then connected to an FPLC (AktaPure-GE) system for elution with 1 mL of elution buffer containing 66.7 μM CA15816 Nb stripper (1 mg/mL) in 10 mM Hepes 7.5, 2.5 mM DTT, 1 mM EDTA, 20 mM Na₂MoO₄, 10% Glycerol at a flowrate flow rate 0.1 mL/min for 6 m L (6 CVs). Regeneration of the column was obtained by 5 CVs of 200 mM Glycine buffer pH 2.3. The purification process was monitored by measuring the absorbance at 280 nm (Protein absorption) and 488 nm (eGFP absorption) (FIGS. 45A-45C). The elution peak was collected in 500 μL fractions and analyzed on an SDS-PAGE gel and using a western blot that was developed with a commercial anti-GFP antibody (FIGS. 45A-45C). The three most prominent bands from the SDS-PAGE analysis of the eluted fractions were cut and analysed by Mass Spectrometry. The bands were identified as human androgen receptor (ARb), Hsp90 and Hsp70 proteins (FIGS. 45A-45C). Hsp70 and Hsp90 are molecular chaperones, known to form a complex with nuclear receptors such as the human androgen receptor in the cytoplasm. This example confirms that NANEX allows fast and easy purification of a native protein-protein complex containing amongst others the eGFP-tagged androgen receptor, Hsp70, Hsp90 and the stripper from transfected HEK293T cells.

### Example 27. High-Throughput Nanobody Exchange Chromatography (NANEX) Purification of Diverse GFP-Tagged Fusion Proteins from Yeast (S. cerevisae) Cell Lysates Using Nb CA15816 as an Immobilized Trapper on Magnetic, Tosyl-Activated Dynabeads®, and Nb CA12760 as a Stripper

Nanobody exchange chromatography (NANEX) is not limited to the purification of proteins on beads packed in columns to mix, and separate solid phases from liquid phases. In example 27 we use magnetic beads in combination with a magnet to mix and exchange solids and liquids in an automated high-throughput setup to purify 12 different proteins in parallel on a KingFisher Flex (ThermoFisher) instrument in standard 96-well plates.

For this experiment, a set of 12 yeast clones was chosen from the yeast GFP clone collection (Huh et al., 2003). Each clone is expressing a different protein as a fusion with GFP and can therefore be purified by NANEX using as a trapper Nb CA15816 and Nb CA12760 as the stripper.

The 96 well format can be used to grow small cultures (1 mL) of yeast cells and offers the possibility to transform, transfect, induce or lyse different cell lines in parallel. This enables fast and high-throughput processes for downstream applications such as protein expression, protein purification, ELISAs, functional assays.

To proof the feasibility of this principle, we performed a NANEX experiment to purify 12 different yeast proteins, expressed in 1 mL cultures, lysed, and purified in parallel in standard 96 well plates. As the solid support, we used tosyl-activated Dynabeads® and coated them with Nb CA15816 (SEQ ID: 3) as a trapper. The purification process was performed in 96-well plates on a KingFisher Flex (ThermoFisher) instrument, including the elution step using Nb CA12760 (SEQ ID: 1) as a stripper.

Given that these clones are not overexpressed but produced at their physiological expression levels, we were able to select house-keeping yeast proteins that are expressed at high levels but also proteins that are expressed at low levels in living cells (FIG. 46).

A selected set of 12 clones expressing GFP-tagged proteins at different levels (FIG. 46) were grown in 96 well deep well plates in 1 mL YPD media for 72 hours. Pellets were lysed for 1 hour in Y-PER™ Plus (ThermoFisher), frozen and spun after defrosting. The recovered lysate served as the source for the purification of the GFP-fused proteins by NANEX with the KingFisher Flex instrument, The Kingfisher is a versatile benchtop automated extraction instrument capable of processing magnetic beads in 96 well format. Tosyl-activated magnetic Dynabeads® were coupled with the trapper Nb CA15816 at a concentration of 40 mg trapper/mg of beads according to the manufacturer's instructions. 5 μL of a 100 mg/mL solution of beads (corresponding to 20 mg of coupled trapper CA15816) was used per well to immobilize and purify proteins from individual clones in a high throughput mode. The parallel purification procedure involved the automated incubation for 30 seconds of the beads with 100 ml of PBS buffer (pre-equilibration), 30 minutes incubation with the lysate (binding of the target), 3 washes of 1 minute with PBS buffer. Proteins were eluted from the magnetic beads in 15 minutes using 40 μL stripper Nb CA12760 at 0.5 mg/mL concentration (33.35 μM) in PBS buffer. From SDS-PAGE and Western blot analysis of the different steps of purification, we conclude that endogenously expressed GFP-fused proteins can be trapped on the Nb CA15816-beads and specifically eluted from these beads by using an appropriate stripper (FIGS. 47A-47G).

### Example 28. Purification of GFP by Nanobody Exchange Chromatography Using Nb CA15816 as an Immobilized Trapper on Magnetic NHS-Activated Agarose Beads Packed in a Sub-Microliter (<1 μL) Microfluidic Column Using Nb CA12760 as a Stripper

High-throughput applications in proteomics and single cell research (amongst others) require protein purification methods that require small devices (downscaling) to separate proteins from small samples. Example 28 illustrates how we designed a microfluidic chip for downscaling our NANEX technology to sub-microliter column volumes (<1 μL) and decreasing the amount of (immobilized) trapper and the amount of stripper required to purify proteins of interest from small samples.

For the purification of GFP on a μ-fluidics device, we covalently immobilized the trapper Nb CA15816 (see example 1) on 50 μl of commercial magnetic NHS-Activated agarose beads (30 μm diameter, Cube Biotech) following the supplier's recommendations. 0.7 μl of these agarose beads was packed into the small chamber (<1 μL) of a custom-made microfluidic chip detailed in FIG. 48. This 30×30×6 mm microfluidic chip was fabricated by milling two 0.420 mm deep channels and a 0.420 mm deep chamber in a 3 mm PMMA plate with a milling machine (Datron model M7). Next, a 3 mm PMMA cover plate was sealed on top with (-)-Butyl L-lactate (Sigma-Aldrich), and an inlet capillary (fused silica, OD 0.360 mm, ID 0.250 mm, CM Scientific) and a smaller diameter outlet capillary (fused silica, OD 0.360 mm, ID 0.075 mm, CM Scientific) were inserted in the channels and cured with adhesive (Norland Optical Adhesive 85) and UV light. The dimensions of the chamber and the channels with inserted capillaries are depicted in FIG. 48. The inlet capillary was connected to a 50 μL syringe (Hamilton Company) operated by a syringe pump (World Precision Instruments model SP100iZ) to inject all the different reagents in the chip (washing buffer, sample, elution buffer and glycine buffer). 0.7 μl of the slurry containing the trapper-functionalized magnetic beads were injected through the wider capillary. As the beads did not pass through the outlet capillary (ID 0.075 mm), the beads were filling the small chamber to constitute a small chromatographic device (<1 μL) with two capillaries functioning as the inlet and the outlet of the column (FIG. 48).

This CA15816 Nb-coupled microfluidic column (0.7 μl CV) was first washed with 50 μl (70 CVs) of washing buffer (PBS) at a flow rate of 10 μL/min. Then, 40 μl of a sample containing the GFP (25 mM Hepes pH7.4, 150 mM NaCl, GFP (0.12 mg/mL)) was injected at 10 μL/min. Next, 90 μl of washing buffer (PBS) was passed over the column at 10 μL/min to remove unbound material. Next, the affinity-trapped GFP was eluted from the μ-fluidic device by applying 52 μl (74 CV) of elution buffer containing the stripper (25 mM HEPES pH7.4, 150 mM NaCl, 26 μg stripper Nb CA12760 (0.5 mg/mL)). After washing with 80 μl washing buffer, regeneration of the column was obtained by injecting 50 μl of the glycine buffer (200 mM glycine buffer pH2.3 at 10 μL/min). The trapping of GFP on the μ-column using Nb CA15816, the stripping of GFP by Nb CA12760 from the μ-column and the regeneration of the μ-column with glycine was monitored using an inverted fluorescence microscope (Olympus IX71 model IX71S1F-3) (FIG. 49). The first wash, flow-through of sample, second wash, stripper eluate, third wash and glycine eluate were also collected from the outlet capillary in 1.5 mL Eppendorf tubes and placed on a blue light transilluminator to visualise the presence or absence of GFP in the different fractions (FIG. 49), confirming that the GFP was indeed trapped and eluted from this microfluidic column following the principles of NANEX and using minimal amounts of the trapper and the stripper.

### Nanobody Expression and Purification

Nanobodies containing a C-terminal His6-tag followed by the EPEA-tag were routinely expressed in and purified from the periplasm of E.coli strain WK6 (Pardon et al., 2014).

Sequence Listing

>SEQ ID NO: 1:
CA12760 GFP-Nb207
(including C-terminal 6xHis + EPEA tag)
>SEQ ID NO: 2:
CA15818 GFP-NbF103A
(mutated residue in bold underlined;
C-term. 6xHis + EPEA)
QVQLVESGGGLVQAGGSLRLSCAASGRTFSTAAMGWFRQAP
GKERDFVAGIYWTVGSTYYADSAKGRFTISRDNAKNTVYLQ
MDSLKPEDTAVYYCAARRRGATLAPTRANEYDYWGQGTQVT
VSSHHHHHHEPEA
>SEQ ID NO: 3:
CA15816 GFP-NbT54A/V55A
(mutated residue in bold underlined;
C-term. 6xHis + EPEA)
QVQLVESGGGLVQAGGSLRLSCAASGRTFSTAAMGWFRQAP
GKERDFVAGIYWAAGSTYYADSAKGRFTISRDNAKNTVYLQ
MDSLKPEDTAVYYCAARRRGFTLAPTRANEYDYWGQGTQVT
VSS HHHHHHEPEA
>SEQ ID NO: 4:
CA15861 GFP-NbT54A/V55A/F103A
(mutated residue in bold underlined;
C-term. 6xHis + EPEA)
QVQLVESGGGLVQAGGSLRLSCAASGRTFSTAAMGWFRQAP
GKERDFVAGIYWAAGSTYYADSAKGRFTISRDNAKNTVYLQ
MDSLKPEDTAVYYCAARRRGATLAPTRANEYDYWGQGTQVT
VSS HHHHHHEPEA

SEQ ID NO: 5: CA15621 Mb_{Nb207}^{cHopQ} (C-term. 6xHis)
SEQ ID NO: 6: CA15616 Mb_{Nb207}^{YgjK} (C-term. 6xHis)
SEQ ID NO: 7: CA4375, EPEA-NbSyn2 (C-term. 6xHis)
SEQ ID NO: 8: CA4394, bivalent EPEA-NbSyn2 EPEA (C-term. 6xHis)
SEQ ID NO: 9: CA13016 Synaptojanin-specific Nb (trapper; C-term. 6xHis+EPEA)
SEQ ID NO: 10: CA13080 Synaptojanin-specific Nb (Stripper; C-term. 6xHis+EPEA)
SEQ ID NO: 11: CA11138 FIXa-specific Nb (Trapped; C-term. 6xHis+EPEA)
SEQ ID NO: 12: CA10304 FIXa-specific Nb (Stripped; C-term. 6xHis+EPEA)
SEQ ID NO: 13: CA10502 FIXa-specific Nb (Trapper2; C-term. 6xHis+EPEA)
SEQ ID NO: 14: CA10309 FIXa-specific Nb (Stripper2; C-term. 6xHis+EPEA)
SEQ ID NO: 15: CA14208 FIXa-specific MegaBody Mb_{NbFIXa}^{cHopQ} (Stripper2; C-term. 6xHis+EPEA)
SEQ ID NO: 16: GFP protein
SEQ ID NO: 17: Helicobacter pylori strain G27 HopQ adhesin domain protein (PDB 5LP2)
SEQ ID NO: 18: CA16047 second GFP Nanobody stripper (including C-terminal 6xHis+EPEA tag)
SEQ ID NO: 19: CA16695 second GFP Nanobody trapper (Y119F) (including C-terminal 6xHis+EPEA tag)
SEQ ID NO: 20: CA16239 GST Nanobody stripper (including C-terminal 6xHis+EPEA tag)
SEQ ID NO: 21: CA16240 GST Nanobody trapper (Y109A) (including C-terminal 6xHis+EPEA tag)
SEQ ID NO: 22: GST (Glutathione 5-transferase)
SEQ ID NO: 23: CA15839 SMT3 Nanobody stripper (including C-terminal 6xHis+EPEA tag)
SEQ ID NO: 24: CA16687 SMT3 Nanobody trapper (D50A) (including C-terminal 6xHis+EPEA tag)
SEQ ID NO: 25: SMT3 (YDR510W)
SEQ ID NO: 26: CA16964 mCherry Nanobody trapper (including C-terminal 6xHis+EPEA tag)
SEQ ID NO: 27: CA17302 mCherry Nanobody stripper (including C-terminal 6xHis+EPEA tag)
SEQ ID NO: 28: CA17341 mCherry Nanobody trapper (CA17302 mutant 1103A) (including C-terminal 6xHis+EPEA tag)
SEQ ID NO: 29: mCherry (FmIH_lectin_mCherry_his) (CA17337)
SEQ ID NO: 30 CA16383 FIX functionalized Nanobody stripper (MegaBody Mb_{CA10309}^{YgjK}, including C-terminal 6xHis+EPEA tag)
SEQ ID NO: 31: CA11143 FIX Nanobody trapper (including C-terminal 6xHis+EPEA tag)
SEQ ID NO: 32: E. coli Ygjk protein (PDB 3WFS)
SEQ ID NO: 33: CA16388 FIX functionalized Nanobody trapper (MegaBody Mb_{CA11143}^{YgjK}, including C-terminal 6xHis+EPEA tag)
SEQ ID NO: 34 CA16607 eGFP-tagged human recombinant glucocorticoid receptor P04150 (eGFP-6His-TEV-GR)
SEQ ID NO: 35: CA16976 eGFP-tagged human recombinant androgen receptor P10275 (eGFP-6His-TEV-ARb)

### Aspects of the disclosure

A method for purification of a target protein comprising the steps of:
mixing a first protein binding agent specifically binding an epitope of a target protein with a sample containing said target protein,
adding to said mix of a) a second protein binding agent, recognizing the same or largely overlapping epitope of said target protein as the first binding agent, to displace the first binding agent from the target protein by specifically binding the target protein, and
collecting the eluting second protein binding agent in complex with the target protein,
   wherein the second protein binding agent comprises an immunoglobulin single variable domain (ISVD) or an active fragment thereof that specifically binds the epitope, and
   wherein the rate constant of dissociation (k_{off} value) of the second protein binding agent is lower as compared to the k_{off} value of the first binding agent.

Said method as described herein, wherein the second protein binding agent has an equal or higher affinity for the epitope, as compared to the first protein binding agent.

Said method as described herein, wherein the K_{D} value for the epitope of the target protein is in the range of 1 μM to 1 nM for the first protein binding agent and in the range of 1 nM to 1 pM for the second protein binding agent.

Said method as described herein, wherein the K_{D} value of the first protein binding agent is 200-5000-fold higher as compared to the K_{D} value of the second protein binding agent.

Said method as any of the methods described herein, wherein the first protein binding agent is immobilized, and the second protein binding agent is in solution.

Said method as any of the methods described herein, wherein the second protein binding agent comprises a functional moiety or a detectable label.

Said method as any of the methods described herein, wherein the sample is a biological sample, a complex mixture, a cellular sample, or an in vitro sample.

Said method as any of the methods described herein, wherein the epitope of the target protein comprises a tag, preferably wherein said tag is selected from the group of GFP, GST, SUMO, Ubiquitin, and EPEA.

Said method as any of the methods described herein, wherein the epitope of the target protein comprises a specific epitope present on a native or endogenous protein.

Said method as any of the methods described herein, wherein the epitope of the target protein comprises a protein binding site on a scaffold protein domain of a MegaBody, preferably said scaffold protein domain comprising HopQ or Ygjk.

Said method as any of the methods described herein, wherein the first protein binding agent is a mutant of the second protein binding agent, with a lower affinity as compared to the second protein binding agent.

Said method as any of the methods described herein, wherein the first protein binding agent comprises an ISVD or an active fragment thereof specifically binding the epitope.

Said method as any of the methods described herein, wherein the ISVD comprises 4 Framework regions (FR) and 3 complementary determining regions (CDR) according to the format of FR1-CDR1-FR2-CDR2-FR3-CDR3-FR4.

Said method of any of the methods described herein, wherein the second protein binding agent is a multivalent form of the first protein binding agent.

Said method of any of the methods described herein, wherein the second protein binding agent is an antigen-binding chimeric protein, in particular a MegaBody™, comprising an ISVD antigen-binding domain specifically binding the epitope and a scaffold protein, preferably a scaffold protein comprising HopQ, Ygjk, or derivatives thereof.

A kit comprising the first and second protein binding agent of the method of any of the methods described herein.

Sad kit, wherein the first protein binding agent is immobilized on a surface.

The kit as described herein , comprising a first and second protein binding agent selected from the group of protein depicted in SEQ ID NO: 1 to 6, or a sequence with at least 70% amino acid identity thereof, wherein the first and second binding agent specifically bind an epitope of GFP.

The method for purification of a target protein, as any of the methods described herein , further comprising the steps of: repeating the steps of the method as any of the methods described herein, using a 3^{rd} and 4^{th} protein binding agent instead of the 1^{st} and 2^{nd} protein binding agents, respectively, wherein said 3^{rd} and 4^{th} binding agent specifically bind a different epitope of said target protein as compared to the epitope for the 1^{st} and 2^{nd} binding agent, and

wherein the 4^{th} protein binding agent has a rate constant of dissociation (k_{off} value) that is lower as compared to the k_{off} value of the 3^{rd} protein binding agent.

A protein complex comprising the second protein binding agent of said method as described herein, or the 4^{th} protein binding agent of the method described herein, and the target protein.

Said protein complex, wherein the target protein comprises a tag selected from the group of GFP, GST, SUMO, Ubiquitin, and EPEA.

Said protein complex, which is crystalline.

Use of any of said protein complexes, for structural analysis, structure-based drug design, mass-spectrometry analysis, or proteomics.

A three-dimensional structural representation at atomic resolution of the protein complex as described herein, with a resolution corresponding to 0.1 to 3 Å.

A crystal comprising the protein complex as described herein, comprising GFP as target protein and GFP-specific Nanobody as second protein binding agent, wherein GFP is depicted in SEQ ID NO: 16 or a sequence with at least 90% identity thereof, and GFP-specific Nanobody is depicted in SEQ ID NO: 1, or a sequence with at least 90% identity thereof, further characterized in that the crystal is in the space group P212121, with the following crystal lattice constants: a=74.497 Å±5%, b=103.450 Å±5%, c=209.774 Å±5%, α=90°, β=90°, γ=90°.

A binding site, consisting of a subset of atomic coordinates, present in the crystal described herein, wherein said binding site consists of the amino acid residues: Pro89, Glu90, Glu111, Lys113, Phe114, Glu115, and Glyl16 of the GFP protein as depicted in SEQ ID NO: 16.

### REFERENCES

Abdiche et al. (2017). Antibodies Targeting Closely Adjacent or Minimally Overlapping Epitopes Can Displace One Another. PLOS ONE, doi:10.1371/journal.pone.0169535.
Aliprandi et al. (2010). The availability of a recombinant anti-SNAP antibody in VHH format amplifies the application flexibility of SNAP-tagged proteins. J Biomed Biotechnol, ID 658954.

Caljon et al. (2015). Description of a Nanobody-based Competitive Immunoassay to Detect Tsetse Fly Exposure. PLOS Neglected Tropical Diseases. doi:10.1371/journal.pntd.0003456.
Capture Select. http://www.captureselect.com.
Götzke et al. (2019). The ALFA-tag is a highly versatile tool for nanobody-based bioscience applications Nat.comms. 10:4403.
Huh W, Falvo J V, Gerke L C, Carroll A S, Howson R W, Weissman J S, and O'Shea E K (2003) Global Analysis of Protein Localization in Budding Yeast Nature 425:686-691.
Javaheri, et al. (2016). Helicobacter pylori adhesin HopQ engages in a virulence-enhancing interaction with human CEACAMs. Nature Microbiology 2, 16189.
Kimple et al. (2015). Overview of Affinity Tags for Protein Purification. Curr Protoc Protein Sci.73: Unit—9.9. doi:10.1002/0471140864.ps0909s73.
Klooster, et al. (2007). Improved anti-IgG and HAS affinity ligands: Clinical application of VHH antibodies technology. J Immunol Meth, 324:1-12.
Pabst et al. (2016). Camelid VHH affinity ligands enable separation of closely related biopharmaceuticals. Biotechnol. J. 2016, 11.
Pardon, et al. (2014). A general protocol for the generation of Nanobodies for structural biology. Nature Protocols. 9: 674-693.
Pratt, W. B. et al. Endocr Rev 18, 306-360 (1997).
Rothbauer et al. (2008). A versatile nanotrap for biochemical and functional studies with fluorescent fusion proteins. Mol Cel Proteomics. 7:282-289.
Tsien R Y (1998) The Green Fluorescent Protein. Annu Rev Biochem 67:509-544.
Verheesen et al. (2003). Beneficial properties of single-domain antibody fragments for application in immunoaffinity purification and immuno-perfusion chromatography. Biochim Biophys Acta, 1624:21-28.

1. A method for purification of a target protein, the method comprising:
a) mixing a first protein binding agent which specifically binds the target protein with a sample containing the target protein,
b) adding to the mix of a), second protein binding agent, wherein the second protein binding agent competes with the first binding agent for binding to the target protein, and by specifically binding the target protein thereby displaces the first binding agent from the target protein, and
c) collecting the eluting second protein binding agent bound to the target protein,
wherein the second protein binding agent comprises an immunoglobulin single variable domain (ISVD) or a functional variant thereof specifically binding the target protein, and
wherein the rate constant of dissociation (k_{off} value) of the second protein binding agent is lower or equal as compared to the k_{off} value of the first binding agent.

2. The method according to claim 1, wherein the second protein binding agent recognizes and binds to the same or a largely overlapping epitope as the first binding agent.

3. The method according to claim 1, wherein the K_{D} value for binding the target protein is in the range of 1 mM to 1 nM for the first protein binding agent and below 1 μM for the second protein binding agent.

4. The method according to claim 3, wherein the K_{D} value of the first protein binding agent is at least 2-fold higher as compared to the K_{D} value of the second protein binding agent.

5. The method according to claim 1, wherein the binding agents specifically bind a tag on the target protein.

6. The method according to claim 1, wherein the binding agents specifically bind a post-translational modification on the target protein.

7. The method according to claim 1, wherein the binding agents specifically bind a scaffold protein domain of the target protein comprising an antigen-binding chimeric protein, wherein the antigen-binding chimeric protein is an ISVD fused to a scaffold protein via at least two sites.

8. The method of any of claims 1 to 7 according to claim 1, wherein the second protein binding agent is a multivalent or multiparatopic form of the first protein binding agent.

9. The method according to claim 1, wherein the first protein binding agent comprises an ISVD or functional variant thereof specifically binding the target protein.

10. The method according to claim 9, wherein the first protein binding agent comprises an ISVD which is mutated in the binding region to the target protein as compared to the second protein binding agent ISVD, and wherein the first protein binding agent has a higher k_{off} as compared to the second protein binding agent.

11. The method according to claim 9, wherein the first and second binding agents comprise an identical ISVD, wherein the ISVD specifically binds the target protein, and preferably with a k_{off} equal or higher than 0.0001 s⁻¹.

12. The method according to claim 1, wherein the first and/or second protein binding agent comprise a functional moiety or a detectable label.

13. The method according to claim 12, wherein the first and/or second protein binding agent comprise a functional moiety characterized in that the functionalized binding agent is an antigen-binding chimeric protein comprising an ISVD fused to a scaffold protein via at least two sites, wherein the ISVD specifically binds the target protein.

14. The method according to claim 1, wherein the first protein binding agent is immobilized, and the second protein binding agent is in solution.

15. The method according to claim 1, wherein the sample is a biological sample, a complex mixture, a cellular sample, or an in vitro sample.

16. The method according to claim 1, further comprising the steps of: repeating steps a) to c) of the method of claims 1-15, using a 3^{rd} and 4^{th} protein binding agent instead of, or in addition to the 1^{st} and 2^{nd} protein binding agents, respectively, wherein the 3^{rd} and 4^{th} binding agent specifically bind a different epitope of the target protein as compared to the epitope for the 1^{st} and 2^{nd} binding agent, and wherein the 4^{th} protein binding agent comprises an ISVD and has a rate constant of dissociation (k_{off} value) that is lower or equal as compared to the k_{off} value of the 3^{rd} protein binding agent.

17. (canceled)

18. A kit comprising a first and second protein binding agent,
wherein the first protein binding agent specifically binds to target protein,
wherein the second protein binding agent competes with the first binding agent for binding to the target protein, and by specifically binding the target protein, thereby displaces the first binding agent from the target protein, and
wherein the second protein binding agent comprises an immunoglobulin single variable domain (ISVD) or a functional variant thereof specifically binding the target protein,
wherein the rate constant of dissociation (k_{off} value) of the second protein binding agent is lower or equal as compared to the k_{off} value of the first binding agent, and
wherein the first protein binding agent is immobilized on a surface and/or provided as a microcolumn or microchip.

19. A kit comprising a first and second protein binding agent,
wherein the first protein binding agent specifically binds to a tag on the target protein,
wherein the second protein binding agent competes with the first binding agent for binding to the tag, and by specifically binding the tag, thereby displaces the first binding agent from the target protein, and
wherein the second protein binding agent comprises an immunoglobulin single variable domain (ISVD) or a functional variant thereof specifically binding the tag,
wherein the rate constant of dissociation (k_{off} value) of the second protein binding agent is lower or equal as compared to the k_{off} value of the first binding agent, and
wherein the binding agents comprise a sequence selected from the group of:
a. SEQ ID NO: 1 to 6, 18 or 19, or a sequence with at least 90% amino acid identity thereof, for specific binding to GFP,
b. SEQ ID NO: 20 and 21, or a sequence with at least 90% amino acid identity thereof, for specific binding to GST,
c. SEQ ID NO: 23 and 24, or a sequence with at least 90% amino acid identity thereof, for specific binding to SMT3,
d. SEQ ID NO: 26, 27 and 28, or a sequence with at least 90% amino acid identity thereof, for specific binding to mCherry,
or comprising any of those sequences without the C-terminal His-EPEA tag, and wherein the first and second protein binding agent cannot be identical when the binding agent has a K_{D} which is below 0.1 nM.

20. (canceled)

21. (canceled)

22. (canceled)

23. (canceled)

24. (canceled)

25. (canceled)

26. The method according to claim 5, wherein the tag is selected from the group of GFP, mCherry, GST, SMT3, and EPEA.

27. The method according to claim 7, wherein the scaffold protein domain comprises HopQ, Ygjk, or a derivative thereof.


SEQUENCE LISTING (35 sequences)

SEQ ID NO: 1 - protein, 136 aa, Artificial Sequence
  CA12760 GFP-Nb207 (including C-terminal 6xHis + EPEA tag)
QVQLVESGGGLVQAGGSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSSHHHHHHEPEA

SEQ ID NO: 2 - protein, 136 aa, Artificial Sequence
  CA15818 GFP-NbF103A
QVQLVESGGGLVQAGGSLRLSCAASGRTFSTAAMGWFRQAPGKERDFVAGIYWTVGSTYYADSAKGRFTISRDNAKNTVYLQMDSLKPEDTAVYYCAARRRGATLAPTRANEYDYWGQGTQVTVSSHHHHHHEPEA

SEQ ID NO: 3 - protein, 136 aa, Artificial Sequence
  CA15816 GFP- NbT54A/V55A
QVQLVESGGGLVQAGGSLRLSCAASGRTFSTAAMGWFRQAPGKERDFVAGIYWAAGSTYYADSAKGRFTISRDNAKNTVYLQMDSLKPEDTAVYYCAARRRGFTLAPTRANEYDYWGQGTQVTVSSHHHHHHEPEA

SEQ ID NO: 4 - protein, 136 aa, Artificial Sequence
  CA15861 GFP- NbT54A/V55A/F103A
QVQLVESGGGLVQAGGSLRLSCAASGRTFSTAAMGWFRQAPGKERDFVAGIYWAAGSTYYADSAKGRFTISRDNAKNTVYLQMDSLKPEDTAVYYCAARRRGATLAPTRANEYDYWGQGTQVTVSSHHHHHHEPEA

SEQ ID NO: 5 - protein, 519 aa, Artificial Sequence
  CA15621 MbNb207cHopQ (C-term. 6xHis)
QVQLVESGGGLVQTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKYGSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSSHHHHHH

SEQ ID NO: 6 - protein, 905 aa, Artificial Sequence
  CA15616 MbNb207YgjK (C-term. 6xHis)
QVQLVESGGGLVYKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKDSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSSHHHHHH

SEQ ID NO: 7 - protein, 132 aa, Artificial Sequence
  CA4375 EPEA-NbSyn2 (C-term. 6xHis)
QVQLQESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSHHHHHH

SEQ ID NO: 8 - protein, 267 aa, Artificial Sequence
  CA4394 bivalent EPEA-NbSyn2 EPEA (C-term. 6xHis)
QVQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSGGGGSGGGSQVQLQESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSHHHHHH

SEQ ID NO: 9 - protein, 136 aa, Artificial Sequence
  CA13016 Synaptojanin-specific Nb (trapper; C-term. 6xHis+EPEA)
  misc_feature (94)..(94): Xaa can be any naturally occurring amino acid
  misc_feature (115)..(115): Xaa can be any naturally occurring amino acid
QVQLVESGGGLVQAGGSLRLSCAASGRTFSGHAMGWFRQAPGKEREFVAAISWNSGITRYIDSVKGRFTISRDDNRNTVYLQMNSLKPEDTAMXYCARNRGIVPGTRPVNDEYDXWGQGTQVTVSSHHHHHHEPEA

SEQ ID NO: 10 - protein, 135 aa, Artificial Sequence
  CA13080 Synaptojanin-specific Nb (Stripper; C-term. 6xHis+EPEA)
QVQLVESGGGLVQAGGSLRLSCAASRFMFDDYIIGWFRQAPGKEREGVSCISNSDGSTNYADSVKGRFTISSDNARNAVYLQMNSLKPEDTAVYYCAAQRAFMTRLCLNIYYAHWGQGTQVTVSSHHHHHHEPEA

SEQ ID NO: 11 - protein, 134 aa, Artificial Sequence
  CA11138 FIXa-specific Nb (Trapper1; C-term. 6xHis+EPEA)
QVQLVESGGGLVQAGGSLRLSCAASERTASNYVMGWFRQAPGKEREFVAFISRTGGSTYYADSVKGRFTVSRDGAKNTVYLQMNTMKPEDTAVYYCAASLYIRNYYEGTNYAYWGQGTQVTVSSHHHHHHEPEA

SEQ ID NO: 12 - protein, 137 aa, Artificial Sequence
  CA10304 FIXa-specific Nb (Stripper1; C-term. 6xHis+EPEA)
QVQLVESGGGLVQAGGSLKLSCAASERTFSGTVMGWFRQPPGKEREFVGDISWSGSITDYADSVKGRFTISRDNAKNTMYLQMDSLKPEDTAVYYCAARVGGYSYIMSRSPADFGSWGQGTQVTVSSHHHHHHEPEA

SEQ ID NO: 13 - protein, 127 aa, Artificial Sequence
  CA10502 FIXa-specific Nb (Trapper2; C-term. 6xHis+EPEA)
QVQLQESGGGLVQAGGSLRLSCAASGRTSRVYGVGWFRQAPGKERELVASISTSGSVTNVADSVKGRFTISRDNAKTTVYLQMNSLKPEDTALYYCAADPHGRPYGWGQGTQVTVSSHHHHHHEPEA

SEQ ID NO: 14 - protein, 130 aa, Artificial Sequence
  CA10309 FIXa-specific Nb (Stripper2; C-term. 6xHis+EPEA)
QVQLVESGGGLVQAGGSLRLSCATSGIGFSIYSMGWYRQAPGKERELVASIAGNEDKDYVESVKGRFTISRDYAKNTLYLEMSGLKFEDTAMYYCAAGLRRGVSLTPYYWGQGTQVTVSSHHHHHHEPEA

SEQ ID NO: 15 - protein, 903 aa, Artificial Sequence
  CA14208 FIXa-specific Megabody MbNbFIXacHopQ (Stripper2; C-term. 6xHis+EPEA)
QVQLVESGGGLVYKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKDSLRLSCATSGIGFSIYSMGWYRQAPGKERELVASIAGNEDKDYVESVKGRFTISRDYAKNTLYLEMSGLKFEDTAMYYCAAGLRRGVSLTPYYWGQGTQVTVSSHHHHHHEPEA

SEQ ID NO: 16 - protein, 238 aa, Artificial Sequence
  GFP protein
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 17 - protein, 428 aa, Heliobacter pylori
MAVQKVKNADKVQKLSDTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKYQQDNQTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNPFRNMGMIASSTTNNGA

SEQ ID NO: 18 - protein, 140 aa, Artificial Sequence
  CA16047 GFP-Nb (including C-terminal 6xHis + EPEA tag)
QVQLQESGGGLVQAGGSLRLSCAASGFTFDGYAIGWFRQAPGKEREGVSCISSSDGSTYYADSVKGRFTISSDNAKNTVYLQMNSLKPEDTAVYYCVAAWSPSTGCGSYYVQRPPGYDYWGQGTQVTVSSHHHHHHEPEA

SEQ ID NO: 19 - protein, 140 aa, Artificial Sequence
  CA16695 GFP-Nb_Y119F (including C-terminal 6xHis + EPEA tag)
QVQLQESGGGLVQAGGSLRLSCAASGFTFDGYAIGWFRQAPGKEREGVSCISSSDGSTYYADSVKGRFTISSDNAKNTVYLQMNSLKPEDTAVYYCVAAWSPSTGCGSYYVQRPPGYDFWGQGTQVTVSSHHHHHHEPEA

SEQ ID NO: 20 - protein, 139 aa, Artificial Sequence
  CA16239 GST-Nb (including C-terminal 6xHis + EPEA tag)
QVQLVESGGGLVQAGGSLRLSCAASERTFSTYNMAWFRQAPGKEREFVAVISWNGGTTNSADSVKGRFTISRDNAKNTVYLQMNSLKPEDTAVYYCAARRVPYLSGTRYYTGEVDYDTWGQGTQVTVSSHHHHHHEPEA

SEQ ID NO: 21 - protein, 139 aa, Artificial Sequence
  CA16240 GST-Nb_Y109A (including C-terminal 6xHis + EPEA tag)
QVQLVESGGGLVQAGGSLRLSCAASERTFSTYNMAWFRQAPGKEREFVAVISWNGGTTNSADSVKGRFTISRDNAKNTVYLQMNSLKPEDTAVYYCAARRVPYLSGTRAYTGEVDYDTWGQGTQVTVSSHHHHHHEPEA

SEQ ID NO: 22 - protein, 218 aa, Artificial Sequence
  Glutathione S-transferase (GST)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 23 - protein, 131 aa, Artificial Sequence
  CA15839 SMT3-Nb (including C-terminal 6xHis + EPEA tag)
QVQLVESGGGSAQTGGSLTLSCTASGSIVSMNSMGWFRRAPWKGREWLGDIGRDGRTSYASSVVGRFHISRDEAKNTVYLQMTRLKPEDAAMYYCAAVPFGDDDVGEWEYWGQGTQVTVSSHHHHHHEPEA

SEQ ID NO: 24 - protein, 131 aa, Artificial Sequence
  CA16687 SMT3-Nb_D50A (including C-terminal 6xHis + EPEA tag)
QVQLVESGGGSAQTGGSLTLSCTASGSIVSMNSMGWFRRAPWKGREWLGAIGRDGRTSYASSVVGRFHISRDEAKNTVYLQMTRLKPEDAAMYYCAAVPFGDDDVGEWEYWGQGTQVTVSSHHHHHHEPEA

SEQ ID NO: 25 - protein, 114 aa, Saccharomyces cerevisiae
MHHHHHHSSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQISSGLEVLFQ

SEQ ID NO: 26 - protein, 134 aa, Artificial Sequence
  CA16964 mCherry Nb (including C-terminal 6xHis + EPEA tag)
QVQLVESGGGLVQAGGSLRLSCAASGRDFSFDGMGWFRQAPGKEREFVAAISWSGLRIGYSDSVKGRFTISRDNAETTVHLQMNSLKPEDTAVYYCATGSHATTKAQLYDYEYWGQGTQVTVSSHHHHHHEPEA

SEQ ID NO: 27 - protein, 136 aa, Artificial Sequence
  CA17302 mCherry Nb (including C-terminal 6xHis + EPEA tag)
QVQLVESGGGLVQAGGSLRLSCVASGLPFSRYNMAWFRQAPGKEREFVADIDWSGDSTYDLDSVKGRFTISRDNAKNTVYLQMSSLLPEDTAVYYCAAHFKFILAQRIKWGDMDYWGKGTQVTVSSHHHHHHEPEA

SEQ ID NO: 28 - protein, 136 aa, Artificial Sequence
  CA17341 mCherry Nb (CA17302 mutant I103A) (including C-terminal 6xHis + EPEA tag)
QVQLVESGGGLVQAGGSLRLSCVASGLPFSRYNMAWFRQAPGKEREFVADIDWSGDSTYDLDSVKGRFTISRDNAKNTVYLQMSSLLPEDTAVYYCAAHFKFALAQRIKWGDMDYWGKGTQVTVSSHHHHHHEPEA

SEQ ID NO: 29 - protein, 415 aa, Artificial Sequence
  mCherry (FmlH_lectin_mCherry_his) (CA17337)
FSCNVDGGSSIGAGTTSVYVNLDPVIQPGQNLVVDLSQHISCWNDYGGWYDTDHINLVQGSAFAGSLQSYKGSLYWNNVTYPFPLTTNTNVLDIGDKTPMPLPLKLYITPVGAAGGVVIKAGEVIARIHMYKIATLGSGNPRNFTWNIISNNSVVMPTGLNASLYKRAGSGLNGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 30 - protein, 901 aa, Artificial Sequence
  CA16383 Factor IX functionalized Nb stripper Megabody MbCA10309YgjK (including C-terminal 6xHis + EPEA tag)
QVQLVESGGGLVKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKSLRLSCATSGIGFSIYSMGWYRQAPGKERELVASIAGNEDKDYVESVKGRFTISRDYAKNTLYLEMSGLKFEDTAMYYCAAGLRRGVSLTPYYWGQGTQVTVSSHHHHHHEPEA

SEQ ID NO: 31 - protein, 133 aa, Artificial Sequence
  CA11143 FIX Nb (including C-terminal 6xHis + EPEA tag)
QVQLVESGGALVQPGGSLRLSCAASGFTFDNYAMNWVRQAPGKGLEWVSGINWNGRNTYYAESVKDRFTISRDNAKNTLYLQMNSLKSEDTAVYYCAKEIITSGGPTGKYDYWGQGTQVTVSSHHHHHHEPEA

SEQ ID NO: 32 - protein, 760 aa, Escherichia coli
NADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQ

SEQ ID NO: 33 - protein, 904 aa, Artificial Sequence
  CA16388 FIX functionalized Nb trapper Megabody MbCA11143YgjK (including C-terminal 6xHis + EPEA tag)
QVQLVESGGGLVKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKSLRLSCAASGFTFDNYAMNWVRQAPGKGLEWVSGINWNGRNTYYAESVKDRFTISRDNAKNTLYLQMNSLKSEDTAVYYCAKEIITSGGPTGKYDYWGQGTQVTVSSHHHHHHEPEA

SEQ ID NO: 34 - protein, 1029 aa, Artificial Sequence
  CA16607 eGFP-tagged human recombinant glucocorticoid receptor P04150 (eGFP-6His-TEV-GR)
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHENLYFQGMDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 35 - protein, 1172 aa, Artificial Sequence
  CA16976 eGFP-tagged human recombinant androgen receptor P10275 (eGFP-6His-TEV-ARb)
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHENLYFQGMEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ